(19) United States
(12) Patent Application Publication
(10) Pub. No.: US 2024/0336591 A1
(43) Pub. Date: Oct. 10, 2024

(54) ORGANIC PYRIDINE-PYRAZOLE COMPOUNDS AND THEIR USES

(71) Applicant: BenevolentAI Cambridge Limited, London (GB)

(72) Inventors: Lee PATIENT, London (GB); Joshua ALMOND-THYNNE, London (GB); Philip James UNSWORTH, London (GB); Adam Christopher Loy YIP, London (GB); Nicola ROBAS, London (GB); Monica Polidoro BELICH, London (GB)

(21) Appl. No.: 18/293,580

(22) PCT Filed: Jul. 29, 2022
(86) PCT No.: PCT/GB22/52009

§ 371 Date: Jan. 30, 2024

(30) Foreign Application Priority Data
Jul. 30, 2021 (GB) 2110990.5

(51) Int. Cl. C07D 401/12; A61K 31/444; A61K 31/4709; A61K 31/498; A61K 31/517; A61K 31/541; A61P 1/04; C07D 409/14; C07D 471/04

(52) U.S. Cl. CPC C07D 401/12; A61K 31/444; A61K 31/4709; A61K 31/498; A61K 31/517; A61K 31/541; A61P 1/04; C07D 409/14; C07D 471/04

(57) ABSTRACT

The present invention relates to compounds of Formulae (IA), (IB), (IIA), and (IIB) or a pharmaceutically acceptable salt, solvate, hydrate, tautomer, optical isomer, N-oxide, and/or prodrug thereof. The invention also relates to the processes for the preparation of those compounds, pharmaceutical compositions comprising those compounds, and the uses of those compounds in treating diseases or conditions associated with inflammatory bowel disease, in particular ulcerative colitis and Crohn's disease.

### TECHNICAL FIELD OF THE INVENTION

The present invention relates to compounds of Formulae (IA), (IB), (IIA), and (IIB) or a pharmaceutically acceptable salt, solvate, hydrate, tautomer, optical isomer, N-oxide, and/or prodrug thereof. The invention also relates to the processes for the preparation of those compounds, pharmaceutical compositions comprising those compounds, and the uses of those compounds in treating diseases or conditions associated with inflammatory bowel disease, in particular ulcerative colitis and Crohn's disease.

### BACKGROUND TO THE INVENTION

Inflammatory bowel diseases are characterised by chronic uncontrolled inflammation affecting the gastro-intestinal tract and leading to multiple symptoms such as weight loss, abdominal pain, recurrent diarrhoea and bleeding. The prevalence of IBD is around 1 in 1000 people in Europe, with higher prevalence and incidence rates observed in westernized and industrialized countries (Loftus E V, Clinical epidemiology of inflammatory bowel disease: Incidence, prevalence, and environmental influences, Gastroenterology. 126(6):1504-17200 (2004)). Peak incidence occurs in the second to fourth decade of life.

Ulcerative colitis (UC) and Crohn's disease (CD) are chronic, immune-mediated disorders that are collectively referred to as inflammatory bowel diseases (IBD). Both CD and UC are characterised by dysregulated, aberrant immune responses of the intestinal mucosa. The goals of treatment for both CD and UC are to achieve symptom control, clinical remission, and to prevent disease progression by eliminating or controlling the inflammatory burden (Rubin, D. T., Ananthakrishnan, et al., Clinical Guideline: Ulcerative Colitis in Adults. Am. J. Gastroenterol. 114, 384-413 (2019)).

UC and CD share many pathologic mechanisms. Antigen-presenting cells, Th1, Th2, T regulatory cells and Th17 T-cells are activated in both UC and CD which results in upregulated expression of multiple proinflammatory cytokines and chemokines (Sartor, R. B. Mechanisms of Disease: pathogenesis of Crohn's disease and ulcerative colitis. Nat Clin Pract Gastr. 3, 390-407 (2006)). There are many common pathways and cytokines that are upregulated in both diseases that play important roles in disease pathology (Ramos, G. P. & Papadakis, K. A. Mechanisms of Disease: Inflammatory Bowel Diseases. Mayo Clin Proc 94, 155-165 (2019)), including the cytokines that the applicant has measured in patient ex-vivo colon biopsies—IL-6 (Mudter, J. & Neurath, M. F. II-6 signaling in inflammatory bowel disease: Pathophysiological role and clinical relevance. Inflamm Bowel Dis 13, 1016-1023 (2007)), IL-8 (Daig, R. et al. Increased interleukin 8 expression in the colon mucosa of patients with inflammatory bowel disease. Gut 38, 216 (1996)), and TNF-α (Friedrich, M., Pohin, M. & Powrie, F. Cytokine Networks in the Pathophysiology of Inflammatory Bowel Disease. Immunity 50, 992-1006 (2019)). Given these shared disease mechanisms between both UC and CD, it is well understood, and supported by a strong rationale, that a therapy effective in UC would be effective in CD, e.g. it has been demonstrated clinically that blockade of TNF alpha by neutralizing monoclonal antibodies treats both active CD and UC (Järnerot, G. et al. Infliximab as Rescue Therapy in Severe to Moderately Severe Ulcerative Colitis: A Randomized, Placebo-Controlled Study. Gastroenterology 128, 1805-1811 (2005); Targan, S. R. et al. A Short-Term Study of Chimeric Monoclonal Antibody cA2 to Tumor Necrosis Factor α for Crohn's Disease. New Engl J Medicine 337, 1029-1036 (1997)).

Genetic studies have also shown that UC and CD share a large number of genes involved in disease pathology, with only a small number of genes specific to each disease (Waterman, M. et al. Distinct and overlapping genetic loci in crohn's disease and ulcerative colitis: Correlations with pathogenesis. Inflamm Bowel Dis 17, 1936-1942 (2011)). A combined genome-wide analysis of CD and UC showed that 110 out of the 163 loci that meet genome-wide significance thresholds are associated with both diseases with 50 of these having indistinguishable effect size in CD and UC, and a large proportion of the remainder showing similar directionality in the 2 diseases (Jostins, L. et al. Host-microbe interactions have shaped the genetic architecture of inflammatory bowel disease. Nature 491, 119-124 (2012)). This degree of sharing of genetic risk suggests that nearly all of the biological mechanisms involved in one disease have some role in the other and therefore that an efficacious treatment for UC would have therapeutic benefit for CD.

There is currently no cure for UC or CD. Therapeutic strategies include interventions on lifestyle habits and medical and surgical treatments. Pharmacological management includes corticosteroids, immunosuppressant agents and anti-tumor necrosis factor (TNF)-α biologics (Baumgart et al., Inflammatory bowel disease: clinical aspects and established and evolving therapies, Lancet. 369(9573), 1641-57, (2007)).

There therefore remains an urgent need for treatments for inflammatory bowel disease, and in particular ulcerative colitis and Crohn's disease.

It is an object of the present invention to provide for treatments which could be used in a range of inflammatory bowel disease conditions. It would be advantageous, if such treatments could be used to treat inflammatory bowel diseases such as ulcerative colitis and Crohn's disease.

### SUMMARY OF THE INVENTION

In a first aspect of the invention there is provided a compound of Formula (IA) or (IB)

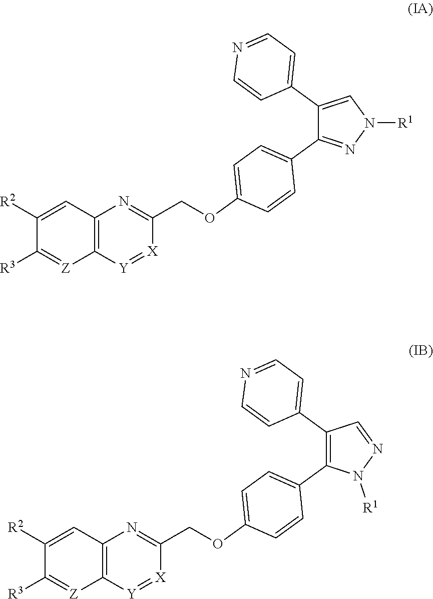

or a pharmaceutically acceptable salt, solvate, hydrate, tautomer, optical isomer,
N-oxide, and/or prodrug thereof, wherein
X is selected from N and CR⁴;
Y is selected from N and CR⁵;
and at least one of CR⁴ and CR⁵ is present;
Z is selected from N and CR⁶—
R¹ is selected from the group consisting of H, C₁-C₆ alkyl and —SO₂R⁷, wherein the C₁-C₆ alkyl is optionally substituted with one or more substituents independently selected from halo, oxo, —NR^{a}R^{b}, —C(O)NR^{a}R^{b}, —C(O)OR^{c}, —OR^{c};
R² and R³ are independently selected from group consisting of H, halo, and C₁-C₆ alkyl, wherein the C₁-C₆ alkyl is optionally substituted with one or more halo atoms;
R⁴ and R⁵ are independently selected from group consisting of H, —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(=NR^{d})R^{e},

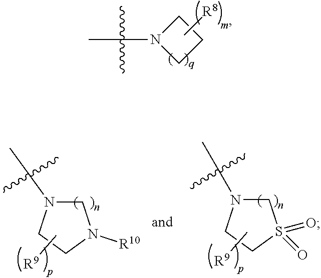

R⁶ is selected from H and C₁-C₆ alkyl, wherein the C₁-C₆ alkyl is optionally substituted with one or more halo atoms;
R⁷ is selected from H and C₁-C₆ alkyl, wherein the C₁-C₆ alkyl is optionally substituted with one or more halo atoms;
R⁸ is selected from C₁-C₆ alkyl, —OH, and —NR^{a}R^{b}, wherein the C₁-C₆ alkyl is optionally substituted with one or more halo atoms;
R⁹ is selected from C₁-C₆ alkyl, —OH, oxo, and —NR^{a}R^{b}, wherein the C₁-C₆ alkyl is optionally substituted with one or more halo atoms;
R¹⁰ is selected from H and C₁-C₆ alkyl, wherein the C₁-C₆ alkyl is optionally substituted with one or more halo atoms;
each R^{a}, R^{b}, R^{c}, R^{d} and R^{e} are independently selected from H and C₁-C₆ alkyl wherein the C₁-C₆ alkyl is optionally substituted with one or more halo atoms,
or R^{a} and R^{b} can be taken together with the nitrogen atom to which they are attached to form a 5- or 6-membered heterocycle, or two R^{e} groups attached to the same atom can be taken together with the atom to which they are attached to form a 5- or 6-membered heterocycle;
m is 0, 1, 2, 3 or 4
n is 1 or 2;
p is 0, 1, 2, 3 or 4; and
q is 0, 1, 2, 3 or 4,
wherein when R¹ is H or optionally substituted C₁-C₆ alkyl, then at least one of R⁴ and R⁵ is present and not H.

In a second aspect of the invention, there is provided a compound of Formula (IIA) or (IIB)

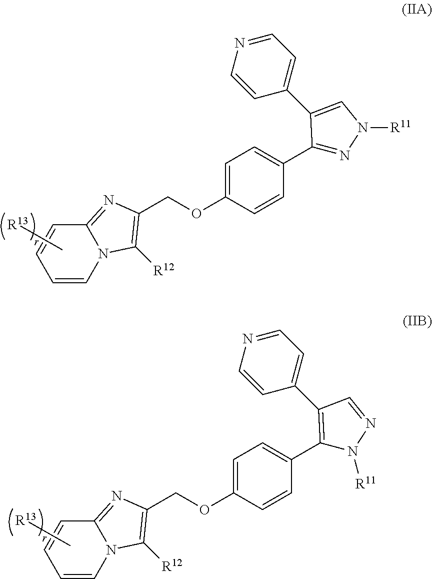

or a pharmaceutically acceptable salt, solvate, hydrate, tautomer, optical isomer, N-oxide, and/or prodrug thereof, wherein
R¹¹ is selected from the group consisting of H, C₁-C₆ alkyl and —SO₂R⁷, wherein the C₁-C₆ alkyl is optionally substituted with one or more substituents independently selected from halo, oxo, —NR^{a}R^{b}, —C(O)NR^{a}R^{b}, —C(O)OR, —OR^{c};
R¹² is selected from the group consisting of H, —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e},

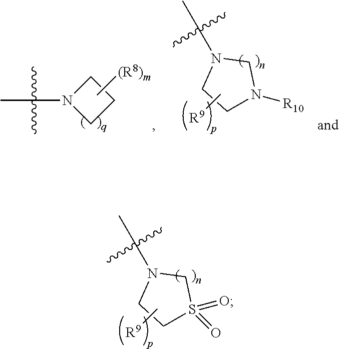

R¹³ is selected from the group consisting of halo, —OR^{f}, and C₁-C₆ alkyl;
R^{f} is selected from the group consisting of H and C₁-C₆ alkyl; and
r is 0, 1, 2, 3, or 4; and
wherein R⁷, R⁸, R⁹, R¹⁰, R^{a}, R^{b}, R^{c}, R^{d}, R^{e}, m, n, p, and q are as defined for the first aspect of the invention, including all preferences etc. thereof.

Compounds of Formulae (IA), (IB), (IIA) and (IIB) are the “compounds of the invention”, or “the compounds”.

The third aspect of the invention provides pharmaceutical compositions comprising a compound of the invention.

The compounds of the invention may inhibit PDE10A at a level suitable to prevent or treat IBD, and in particular ulcerative colitis and/or Crohn's disease, for the reasons set out below.

It has been surprisingly and advantageously found that the selective inhibition of PDE10A with a small molecule inhibitor reduces inflammatory cytokine levels in colon samples from IBD patients and therefore this represents an unexpected and promising treatment for inflammatory bowel diseases, and in particular, ulcerative colitis and Crohn's disease.

Cyclic nucleotide phosphodiesterases (PDEs) are a family of enzymes that catalyse the degradation of the cyclic nucleotide second messengers cyclic adenosine monophosphate (cAMP) and cyclic guanosine monophosphate (cGMP). Intracellular levels of the cAMP and cGMP are regulated by both their rates of synthesis (by adenylate cyclases and guanylate cyclases respectively) and their hydrolysis by phosphodiesterases. By regulating the duration and amplitude of the cAMP and cGMP second messenger signals, PDEs play critical regulatory roles in signal transduction.

There are 11 different PDE subtypes (PDE1 to PDE11), each encoding PDEs with unique substrate specificities, kinetics, allosteric regulators, tissue-expression profiles, and pharmacological sensitivities. PDE10A is able to hydrolyse both cAMP and cGMP. PDE10A hydrolyzes cAMP with a Kₘ of 0.05 μM and cGMP with a Kₘ of 3 μM. Although PDE10A has a lower Kₘ for cAMP, the Vₘₐₓ ratio cGMP/cAMP is 4.7 indicating a higher specific activity for cGMP. Taken together this suggests that PDE10A is a cAMP-inhibited cGMP phosphodiesterase.

In normal tissue, PDE10A has a restricted expression pattern. High PDE10ARNA levels are detected only in the striatum (caudate nucleus and putamen) of the brain, and the testes (Fujishige K, Kotera J, Michibata H, Yuasa K, Takebayashi S, Okumura K, Omori K. Cloning and characterization of a novel human phosphodiesterase that hydrolyzes both cAMP and cGMP (PDE10A). J Biol Chem. 274, 18438-18445 (1999)). To date inhibitors of PDE10A have mainly been investigated for neurological conditions including schizophrenia and Parkinson's disease (Geerts H, Spiros A, Roberts P. Phosphodiesterase 10 inhibitors in clinical development for CNS disorders. Expert Rev Neurother. 17(6), 553-560 (2017)). PDE10A has not been investigated extensively for inflammation. A search of the literature identified one paper (García A M et al. Targeting PDE10A GAF Domain with Small Molecules: A Way for Allosteric Modulation with Anti-Inflammatory Effects. Molecules., 1472, 22(9), 2017), that described inhibition of LPS-induced nitrite release from the Raw 264.7 macrophage cell line by a PDE10A inhibitor i.e. in a transformed mouse cell line rather than human primary cells. The authors ascribed the effect seen to the cAMP hydrolytic activity of PDE10A rather than its cGMP activity.

The present inventors have surprisingly found that PDE10A inhibitors described herein can reduce the levels of inflammatory cytokines, that are a hallmark of IBD, in colon biopsies taken from IBD patients, and therefore represent a new therapeutic opportunity for the treatment of these diseases.

The inflammatory bowel diseases may comprise ulcerative colitis and/or Crohn's disease. It is well understood that any treatment for ulcerative colitis is likely to be suitable to treat Crohn's disease, and vice-versa. This is demonstrated for the present compounds in the Examples below.

The present invention provides compounds, that may be PDE10A inhibitors, for use in the prevention and/or treatment of inflammatory bowel disease. Suitably the inflammatory bowel disease is selected from ulcerative colitis and/or Crohn's disease. This is the fourth aspect of the invention.

### DESCRIPTION OF FIGURES

FIG. 1 contains plots showing RNA expression of PDE10A in normal tissue. The plots represent baseline gene expression of PDE10A and GUCY2C (guanylate cyclase 2C) in healthy samples based on GTEx data, where the X-axis represents tissue, y-axis represents log₂ transformed expression.

FIG. 2 contains volcano plots showing differential RNA expression of PDE10A and GUCY2C. The volcano plots show differential gene expression for a selected comparison, where the x-axis represents log fold change (FC) and y-axis represents log₁₀ transformed adjusted p-value (FDR). Horizontal dotted line is FDR=0.05 threshold and values above the dotted line are considered significant. Values to the right of the central axis indicate upregulation, values to the left of the central axis indicate down regulation. The OmicSoft differential expression datasets used in the analysis were as follows: colonic mucosa -OmicSoft Project names: GSE14580, GSE16879, GSE36807, GSE59071, GSE65114, GSE73661; colon—OmicSoft Project names: GSE10191, GSE10616, GSE6731, GSE9686.

FIG. 3 is a graph showing the effect of a PDE10A inhibitor, PF-02545920, on isolated human neutrophil activation in response to IL-8.

FIG. 4 contains graphs showing PF-02545920 and TAK-063 inhibiting the release of inflammatory cytokines IL-6 and IL-8 in ex-vivo cultures of colon biopsy samples from a UC patient (UC donor 1). (A) Effect of PDE10A inhibitors on IL-6 levels, (B) Effect of PDE10A inhibitors on IL-8 levels, (n=2; Mean±SD), wherein Pred=prednisolone, Tofa=tofacitinib.

FIG. 5 contains graphs showing PF-02545920 and TAK-063 inhibiting the release of inflammatory cytokines IL-6 and IL-8 in ex-vivo cultures of colon biopsy samples from a UC patient (UC donor 2). (A) Effect of PDE10A inhibitors on IL-6 levels, (B) Effect of PDE10A inhibitors on IL-8 levels, (n=2; Mean±SD), wherein Pred=prednisolone, Tofa=tofacitinib.

FIGS. 6 to 9 contain graphs showing the effect of the compound of Example 4 (FIG. 6A), Example 9 (FIG. 6B), Example 10 (FIG. 7A), Example 11 (FIG. 7B), Example 17 (FIG. 8A), Example 19 (FIG. 8B), and Example 20 (FIG. 9) on inflammatory cytokine release from ex-vivo ulcerative colitis colon tissue (UC donor 3).

FIG. 10 contains graphs showing PF-02545920 (1 μM) inhibiting release of the inflammatory cytokine TNFα in ex-vivo cultures of inflamed colon tissue obtained from surgical resection from treatment-refractory UC patients. (A) UC donor 4, (B) UC donor 5 (n=5; Mean±SD; *p<0.05).

FIG. 11 shows that PF-2545920 inhibits the spontaneous release of inflammatory cytokines IL-6 and IL-8 in ex-vivo cultures of inflamed CD colon tissue. Graph (A) CD donor 1, graph (B) CD donor 2 (n=2; Mean±SD).

### DETAILED DESCRIPTION OF THE INVENTION

The applicant has found that certain compounds may be used to prevent and/or treat diseases or conditions susceptible to PDE10A inhibition. In a first aspect of the invention there is provided a compound of Formula (IA) or (IB)

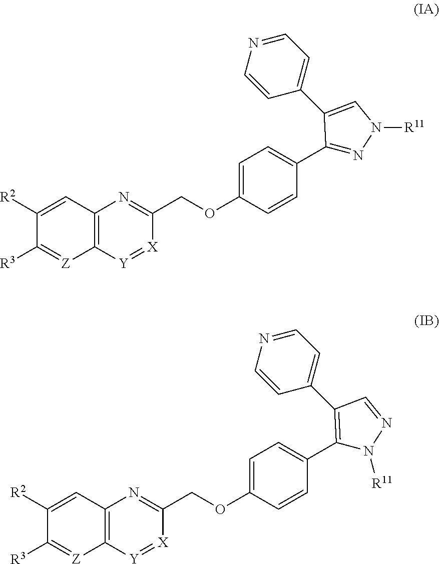

or a pharmaceutically acceptable salt, solvate, hydrate, tautomer, optical isomer,
N-oxide, and/or prodrug thereof, wherein
X is selected from N and CR⁴;
Y is selected from N and CR⁵;
and at least one of CR⁴ and CR⁵ is present;
Z is selected from N and CR⁶—
R¹ is selected from the group consisting of H, C₁-C₆ alkyl and —SO₂R⁷, wherein the C₁-C₆ alkyl is optionally substituted with one or more substituents independently selected from halo, oxo, —NR^{a}R^{b}, —C(O)NR^{a}R^{b}, —C(O)OR, —OR^{c};
R² and R³ are independently selected from group consisting of H, halo, and C₁-C₆ alkyl, wherein the C₁-C₆ alkyl is optionally substituted with one or more halo atoms;
R⁴ and R⁵ are independently selected from group consisting of H, —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(=NR^{d})R^{e},

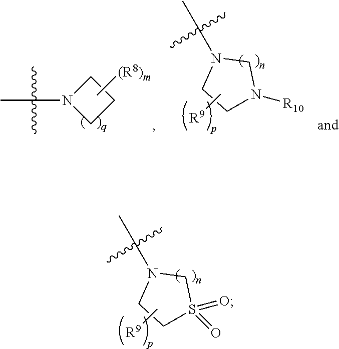

R⁶ is selected from H and C₁-C₆ alkyl, wherein the C₁-C₆ alkyl is optionally substituted with one or more halo atoms;
R⁷ is selected from H and C₁-C₆ alkyl, wherein the C₁-C₆ alkyl is optionally substituted with one or more halo atoms;
R⁸ is selected from C₁-C₆ alkyl, —OH, and —NR^{a}R^{b}, wherein the C₁-C₆ alkyl is optionally substituted with one or more halo atoms;
R⁹ is selected from C₁-C₆ alkyl, —OH, oxo, and —NR^{a}R^{b}, wherein the C₁-C₆ alkyl is optionally substituted with one or more halo atoms;
R¹⁰ is selected from H and C₁-C₆ alkyl, wherein the C₁-C₆ alkyl is optionally substituted with one or more halo atoms;
each R^{a}, R^{b}, R^{c}, R^{d} and R^{e} are independently selected from H and C₁-C₆ alkyl wherein the C₁-C₆ alkyl is optionally substituted with one or more halo atoms,
or R^{a} and R^{b} can be taken together with the nitrogen atom to which they are attached to form a 5- or 6-membered heterocycle,
or two R^{e} groups attached to the same atom can be taken together with the atom to which they are attached to form a 5- or 6-membered heterocycle;
m is 0, 1, 2, 3 or 4
n is 1 or 2;
p is 0, 1, 2, 3 or 4; and
q is 0, 1, 2, 3 or 4,
wherein when R¹ is H or optionally substituted C₁-C₆ alkyl, then at least one of R⁴ and R⁵ is present and not H.

Compounds of Formulae (IA) and (IB) differ in respect of the location of group R¹ on the pyrazole. Compounds of both formulae may inhibit PDE10A, however, the compounds of Formula (IA) are preferred due to the additional advantages that they provide. Therefore, a feature of the first aspect of the invention is that the compound is of Formula (IA).

In the broadest sense of the invention, X is selected from N and CR⁴, and Y is selected from N and CR⁵. However, compounds in which both X and Y and N are not within the scope of the application. This means that at least one of CR⁴ and CR⁵ is present. It may be that both CR⁴ and CR⁵ are present in the compound. The compounds of Formulae (IA) and (IB) may therefore comprise the following groups.

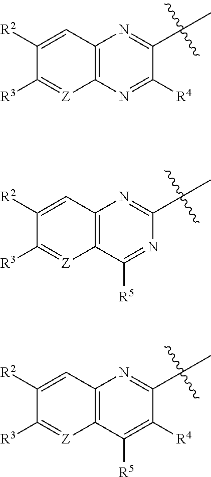

Compounds in which X is CR⁴ and Y is CR⁵ are particularly preferred.

Certain substituents on the compounds of Formulae (IA) and (IB) are preferable. In one feature, at least one of R⁴ and R⁵ is present and is selected from the group consisting of —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e},

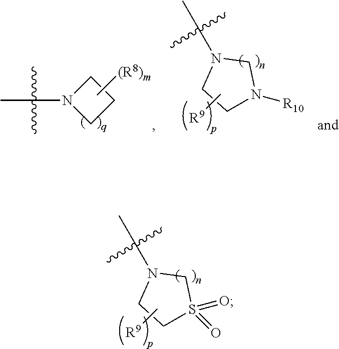

With regard to group

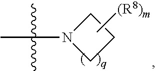

it is preferable that it is a 4- to 6-membered ring. Examples or such rings include, but are not limited to

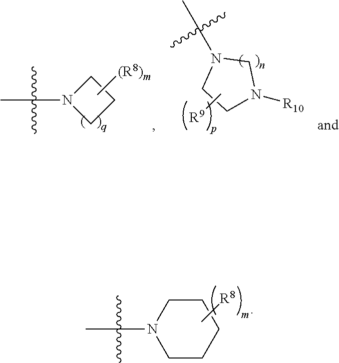

With regard to groups

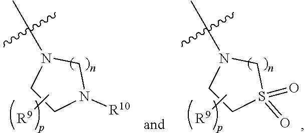

it is preferred that n is 2, and therefore the groups are

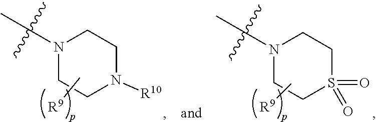

respectively.

In view of this, a preferred feature of the first aspect of the invention is that at least one of R⁴ and R⁵ is present and is selected from the group consisting of —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(=NR^{d})R^{e},

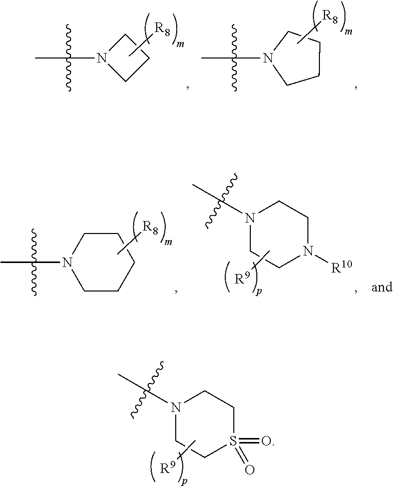

More specifically for group X, is it preferable that it is selected from N and CR⁴, wherein R⁴ is selected from H, —C(O)OR^{c}, —C(O)N(R^{d})SO₂R^{e}; —C(O)N═S(O)R^{e}₂,

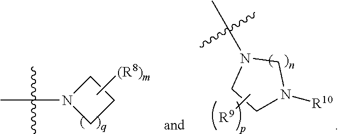

It is more preferable that X is selected from N and CR⁴, wherein R⁴ is selected from H, —C(O)OR^{c}, —C(O)N(R^{d})SO₂R^{e}; —C(O)N═S(O)R^{e}₂,

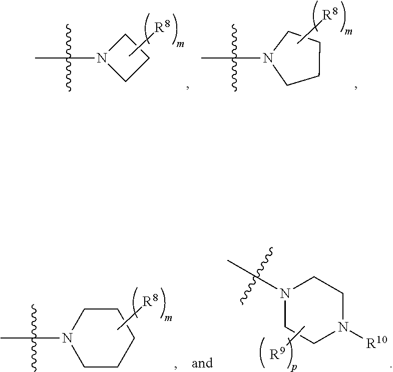

It is most preferable that X is selected from N and CR⁴, and R⁴ is selected from H, —C(O)OH, —C(O)NHSO₂Me, —C(O)NMeSO₂Me, —C(O)N═S(O)Me₂,

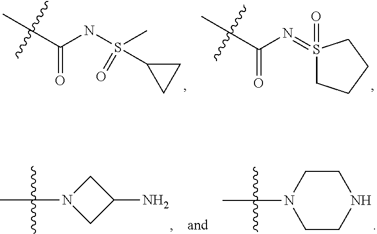

More specifically for group Y, is it preferable that it is selected from N and CR⁵, and R⁵ is selected from H, —C(O)OH, —C(O)N(Me)SO₂Me, —C(O)N═S(O)Me₂, —N═S(O)Me₂, —NHC(O)N═S(O)Me₂, —NHC(O)NHMe, —NHSO₂Me, —S(O)(═NH)Me,

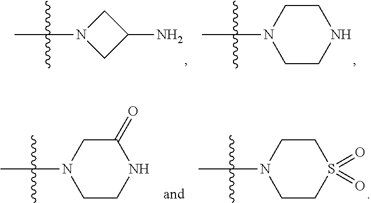 [SMILES: *N1CC(N)C1 ; *N1CCNCC1 ; *N1CCNC(=O)C1 ; *N1CCS(=O)(=O)CC1]

When either or both of groups R⁴ and R⁵ comprise substituents R^{d}, it is preferred that R^{d} is selected from H and Me. When either or both of groups R⁴ and R⁵ comprise one or more substituents R^{e}, it is preferred that each R^{e} may be independently Me, cyclopropyl, or two R^{e} groups attached to the same atom may be taken together with the atom to which they are attached to form a 5- or 6-membered heterocycle. In these cases, it is preferable that R^{d} is selected from H and Me, and each R^{e} is independently Me, cyclopropyl, or two R^{e} groups attached to the same atom may be taken together with the atom to which they are attached to form a 5- or 6-membered heterocycle.

When two R^{e} groups attached to the same atom are taken together with the atom to which they are attached to form a 5- or 6-membered heterocycle, they may form groups such as, but not limited to, the following.

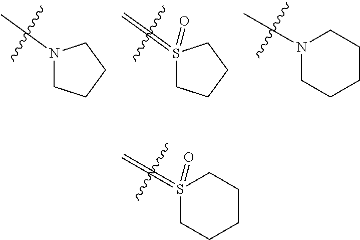 [SMILES: *N1CCCC1 ; *=S1(=O)CCCC1 ; *N1CCCCC1 ; *=S1(=O)CCCCC1]

R² and R³ are independently selected from group consisting of H, halo, and C₁-C₆ alkyl, wherein the C₁-C₆ alkyl is optionally substituted with one or more halo atoms. It is preferable that R² and R³ are selected from H, F, and Me. More specifically, in particularly useful compounds, R² may be selected from H and F, and R³ may be selected from H, F and Me. In one feature of the first aspect of the invention, R² is H, R³ is H, or both R² and R³ are H.

With regard to R¹, it may be preferable that it is selected from the group consisting of H, C₁-C₃ alkyl and —SO₂Me, wherein the C₁-C₃ alkyl is optionally substituted with one or more substituents independently selected from halo and —C(O)OH. More preferably, R¹ may be selected from the group consisting of H, Me, Et, —CH₂CF₃, —CH₂C(O)OH, cyclopropyl, and —SO₂Me. These compounds may be particularly advantageous.

Z is selected from N and CR⁶, wherein R⁶ is selected from H and C₁-C₆ alkyl, wherein the C₁-C₆ alkyl is optionally substituted with one or more halo atoms. The compounds of Formula (IA) and (IB) may therefore comprise the following groups.

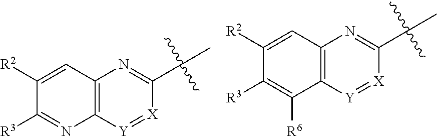

It is preferable that Z is selected from N and CR⁶, wherein the R⁶ is selected from H, F, Cl and Me. It is more preferable that Z is CR⁶, in particular wherein Z is CR⁶, wherein the R⁶ is selected from H, F, Cl and Me.

In view of the above, the compounds of Formulae (IA) and (IB) may therefore comprise the following groups.

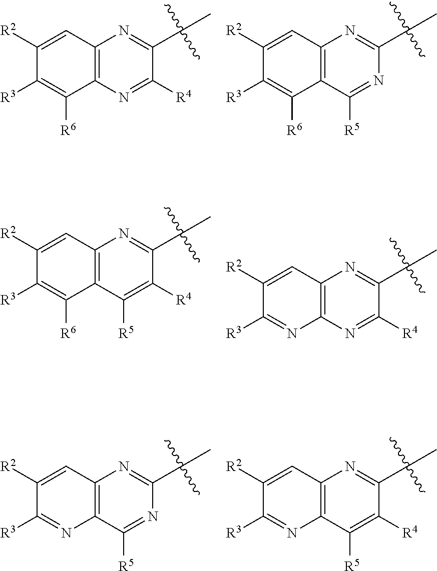

In a particularly preferred feature of the first aspect of the invention, X is selected from N and CR⁴, wherein R⁴ is selected from H, —C(O)OR^{c}, —C(O)N(R^{d})SO₂R^{e}; —C(O)N═S(O)R^{e}₂,

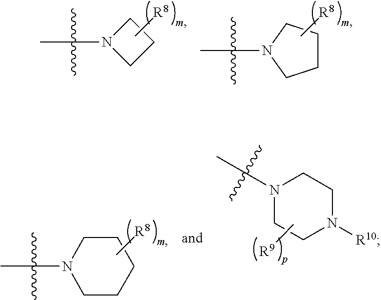

Y is selected from N and CR⁵, and R⁵ is selected from H, —C(O)OH, —C(O)N(Me)SO₂Me, —C(O)N═S(O)Me₂, —N═S(O)Me₂, —NHC(O)N═S(O)Me₂, —NHC(O)NHMe, —NHSO₂Me, —S(O)(═NH)Me,

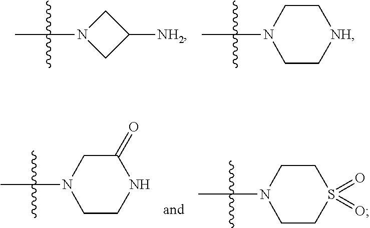 [SMILES: *N1CC(N)C1 ; *N1CCNCC1 ; *N1CCNC(=O)C1 ; *N1CCS(=O)(=O)CC1]

and at least one of CR⁴ and CR⁵ is present;
Z is selected from N and CR⁶, wherein the R⁶ is selected from H, F, Cl and Me;
R¹ is selected from the group consisting of H, C₁-C₃ alkyl and —SO₂Me, wherein the C₁-C₃ alkyl is optionally substituted with one or more substituents independently selected from halo and —C(O)OH;
R² is selected from H and F;
R³ is selected from H, F and Me;
R⁸ is selected from C₁-C₆ alkyl, —OH, and —NR^{a}R^{b}, wherein the C₁-C₆ alkyl is optionally substituted with one or more halo atoms;
R⁹ is selected from C₁-C₆ alkyl, —OH, oxo, and —NR^{a}R^{b}, wherein the C₁-C₆ alkyl is optionally substituted with one or more halo atoms;
R¹⁰ is selected from H and C₁-C₆ alkyl, wherein the C₁-C₆ alkyl is optionally substituted with one or more halo atoms;
each R^{a}, R^{b}, R^{c} are independently selected from H and C₁-C₆ alkyl wherein the C₁-C₆ alkyl is optionally substituted with one or more halo atoms,
or R^{a} and R^{b} can be taken together with the nitrogen atom to which they are attached to form a 5- or 6-membered heterocycle,
each R^{e} is Me, and R^{d} is selected from H and Me, or two R^{e} groups attached to the same atom can be taken together with the atom to which they are attached to form a 5- or 6-membered heterocycle;
m is 0, 1, 2, 3 or 4
p is 0, 1, 2, 3 or 4; and
wherein when R¹ is H or optionally substituted C₁-C₆ alkyl, then at least one of R⁴ and R⁵ is present and not H.

In a more preferred feature of the first aspect of the invention, X is selected from N and CR⁴, and R⁴ is selected from H, —C(O)OH, —C(O)NHSO₂Me, —C(O)NMeSO₂Me, —C(O)N═S(O)Me₂,

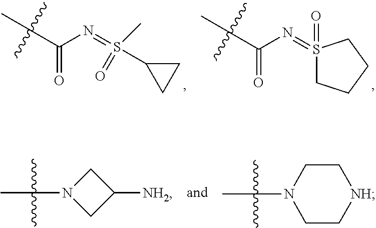

Y is selected from N and CR⁵, and R⁵ is selected from H, —C(O)OH, —C(O)N(Me)SO₂Me, —C(O)N═S(O)Me₂, —N═S(O)Me₂, —NHC(O)N═S(O)Me₂, —NHC(O)NHMe, —NHSO₂Me, —S(O)(═NH)Me,

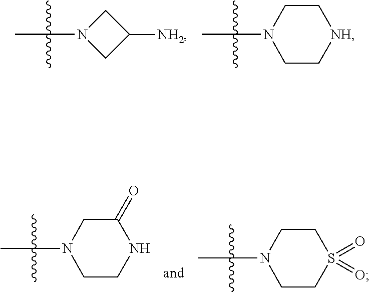 [SMILES: *N1CC(N)C1 ; *N1CCNCC1 ; *N1CCNC(=O)C1 ; *N1CCS(=O)(=O)CC1]

and at least one of CR⁴ and CR⁵ is present;
Z is selected from N and CR⁶, wherein the R⁶ is selected from H, F, Cl and Me;
R¹ is selected from the group consisting of H, Me, Et, —CH₂CF₃, CH₂C(O)OH, cyclopropyl, and —SO₂Me;
R² is selected from H and F;
R³ is selected from H, F and Me; and
wherein when R¹ is H or optionally substituted C₁-C₆ alkyl, then at least one of R⁴ and R⁵ is present and not H.

Notwithstanding the above, preferred compounds of the first aspect of the invention are
2-[[4-[2-Methyl-4-(4-pyridyl)pyrazol-3-yl]phenoxy]methyl]quinoline-4-carboxylic acid
2-[[4-[2-Methyl-4-(4-pyridyl)pyrazol-3-yl]phenoxy]methyl]quinoline-3-carboxylic acid
2-[[4-[1-Methyl-4-(4-pyridyl)pyrazol-3-yl]phenoxy]methyl]quinoline-3-carboxylic acid
Ammonium 2-[[4-[1-methyl-4-(4-pyridyl)pyrazol-3-yl]phenoxy]methyl]quinoline-4-carboxylate
2-[4-(4-Pyridyl)-3-[4-(2-quinolylmethoxy)phenyl]pyrazol-1-yl]acetic acid
2-[[4-[1-Methylsulfonyl-4-(4-pyridyl)pyrazol-3-yl]phenoxy]methyl]quinoline
N-[Dimethyl(oxo)-λ6-sulfanylidene]-5-methyl-2-[[4-[1-methyl-4-(4-pyridyl)pyrazol-3-yl]phenoxy]methyl]quinoline-3-carboxamide
N-[Dimethyl(oxo)-λ6-sulfanylidene]-2-[[4-[4-(4-pyridyl)-1-(2,2,2-trifluoroethyl)pyrazol-3-yl]phenoxy]methyl]quinoline-4-carboxamide
2-[[4-[1-Methyl-4-(4-pyridyl)pyrazol-3-yl]phenoxy]methyl]-N-methylsulfonyl-quinoline-3-carboxamide
N-[Dimethyl(oxo)-λ6-sulfanylidene]-2-[[4-[1-methyl-4-(4-pyridyl)pyrazol-3-yl]phenoxy]methyl] quinoline-3-carboxamide
N-[Dimethyl(oxo)-λ6-sulfanylidene]-2-[[4-[1-methyl-4-(4-pyridyl)pyrazol-3-yl]phenoxy]methyl] quinoline-4-carboxamide
N-[Dimethyl(oxo)-λ6-sulfanylidene]-2-[[4-[4-(4-pyridyl)-1H-pyrazol-3-yl]phenoxy]methyl] quinoline-4-carboxamide
2-[[4-[1-Methyl-4-(4-pyridyl)pyrazol-3-yl]phenoxy]methyl]-N-(1-oxothiolan-1-ylidene)quinoline-3-carboxamide
N-(Cyclopropyl-methyl-oxo-λ6-sulfanylidene)-2-[[4-[1-methyl-4-(4-pyridyl)pyrazol-3-yl]phenoxy]methyl] quinoline-3-carboxamide
N-Methyl-2-[[4-[1-methyl-4-(4-pyridyl)pyrazol-3-yl]phenoxy]methyl]-N-methylsulfonyl-quinoline-3-carboxamide
N-Methyl-2-[[4-[1-methyl-4-(4-pyridyl)pyrazol-3-yl]phenoxy]methyl]-N-methylsulfonyl-quinoline-4-carboxamide
N-[Dimethyl(oxo)-λ6-sulfanylidene]-2-[[4-[1-methyl-4-(4-pyridyl)pyrazol-3-yl]phenoxy]methyl]quinazoline-4-carboxamide
N-[Dimethyl(oxo)-λ6-sulfanylidene]-3-[[4-[1-methyl-4-(4-pyridyl)pyrazol-3-yl]phenoxy]methyl] quinoxaline-2-carboxamide
N-[Dimethyl(oxo)-λ6-sulfanylidene]-2-[[4-[1-methyl-4-(4-pyridyl)pyrazol-3-yl]phenoxy]methyl]-1,5-naphthyridine-3-carboxamide
N-[Dimethyl(oxo)-λ6-sulfanylidene]-7-fluoro-2-[[4-[1-methyl-4-(4-pyridyl)pyrazol-3-yl]phenoxy]methyl]quinoline-3-carboxamide
N-[Dimethyl(oxo)-λ6-sulfanylidene]-6-fluoro-2-[[4-[1-methyl-4-(4-pyridyl)pyrazol-3-yl]phenoxy]methyl]quinoline-3-carboxamide
N-[Dimethyl(oxo)-λ6-sulfanylidene]-5-fluoro-2-[[4-[1-methyl-4-(4-pyridyl)pyrazol-3-yl]phenoxy]methyl]quinoline-3-carboxamide
N-[Dimethyl(oxo)-λ6-sulfanylidene]-6-methyl-2-[[4-[1-methyl-4-(4-pyridyl)pyrazol-3-yl]phenoxy]methyl]quinoline-3-carboxamide
N-[Dimethyl(oxo)-λ6-sulfanylidene]-6-fluoro-2-[[4-[1-methyl-4-(4-pyridyl)pyrazol-3-yl]phenoxy]methyl]quinoline-4-carboxamide
5-Chloro-N-[Dimethyl(oxo)-λ6-sulfanylidene]-2-[[4-[1-methyl-4-(4-pyridyl)pyrazol-3-yl]phenoxy]methyl]quinoline-4-carboxamide
N-[Dimethyl(oxo)-λ6-sulfanylidene]-2-[[4-[1-ethyl-4-(4-pyridyl)pyrazol-3-yl]phenoxy]methyl]quinoline-4-carboxamide
2-[[4-[1-Cyclopropyl-4-(4-pyridyl)pyrazol-3-yl]phenoxy]methyl]-N-[dimethyl(oxo)-λ6-sulfanylidene]quinoline-4-carboxamide
2-[[4-[4-(4-Pyridyl)-1H-pyrazol-3-yl]phenoxy]methyl]quinoline-3-carboxylic acid
2-[[4-[4-(4-Pyridyl)-1H-pyrazol-3-yl]phenoxy]methyl]quinoline-4-carboxylic acid
N-[Dimethyl(oxo)-λ6-sulfanylidene]-2-[[4-[4-(4-pyridyl)-1H-pyrazol-3-yl]phenoxy]methyl]quinoline-3-carboxamide
Dimethyl-oxo-[[2-[[4-[4-(4-pyridyl)-1H-pyrazol-3-yl]phenoxy]methyl]-4-quinolyl]imino]-λ6-sulfane
Imino-methyl-oxo-[2-[[4-[4-(4-pyridyl)-1H-pyrazol-3-yl]phenoxy]methyl]-4-quinolyl]-λ6-sulfane
N-[2-[[4-[1-Methyl-4-(4-pyridyl)pyrazol-3-yl]phenoxy]methyl]-4-quinolyl]methanesulfonamide
1-Methyl-3-[2-[[4-[1-methyl-4-(4-pyridyl)pyrazol-3-yl]phenoxy]methyl]-4-quinolyl]urea
1-[Dimethyl(oxo)-λ6-sulfanylidene]-3-[2-[[4-[1-methyl-4-(4-pyridyl)pyrazol-3-yl]phenoxy]methyl]-4-quinolyl]urea
4-[2-[[4-[1-Methyl-4-(4-pyridyl)pyrazol-3-yl]phenoxy]methyl]quinazolin-4-yl]-1,4-thiazinane 1,1-dioxide
4-[2-[[4-[1-Methyl-4-(4-pyridyl)pyrazol-3-yl]phenoxy]methyl]quinazolin-4-yl]piperazin-2-one
1-[2-[[4-[1-Methyl-4-(4-pyridyl)pyrazol-3-yl]phenoxy]methyl]quinazolin-4-yl]azetidin-3-amine
2-[[4-[1-Methyl-4-(4-pyridyl)pyrazol-3-yl]phenoxy]methyl]-4-piperazin-1-yl-quinazoline
1-[3-[[4-[1-Methyl-4-(4-pyridyl)pyrazol-3-yl]phenoxy]methyl]quinoxalin-2-yl]azetidin-3-amine
2-[[4-[1-Methyl-4-(4-pyridyl)pyrazol-3-yl]phenoxy]methyl]-3-piperazin-1-yl-quinoxaline
   or a pharmaceutically acceptable salt, solvate, hydrate, tautomer, optical isomer, N-oxide, and/or prodrug thereof.

In a second aspect of the invention, there is provided a compound of Formula (IIA) or (IIB)

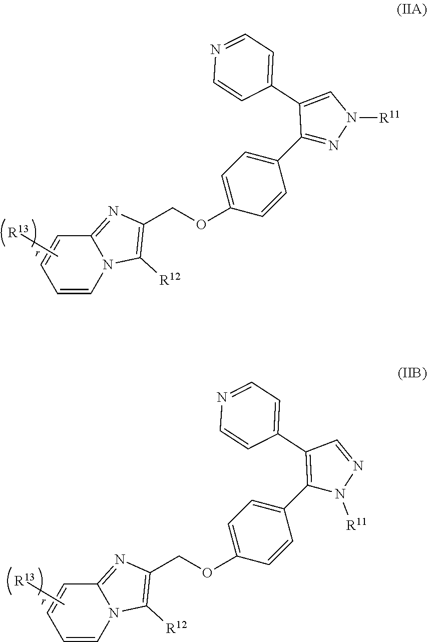

or a pharmaceutically acceptable salt, solvate, hydrate, tautomer, optical isomer, N-oxide, and/or prodrug thereof, wherein
R¹¹ is selected from the group consisting of H, C₁-C₆ alkyl and —SO₂R⁷, wherein the C₁-C₆ alkyl is optionally substituted with one or more substituents independently selected from halo, oxo, —NR^{a}R^{b}, —C(O)NR^{a}R^{b}, —C(O)OR^{c}, —OR^{c}(preferably R¹¹ is selected from the group consisting of H and C₁-C₆ alkyl, wherein the C₁-C₆ alkyl is optionally substituted with one or more halo, wherein it is preferable that the one or more halo is one or more F);
R¹² is selected from the group consisting of H, —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e},

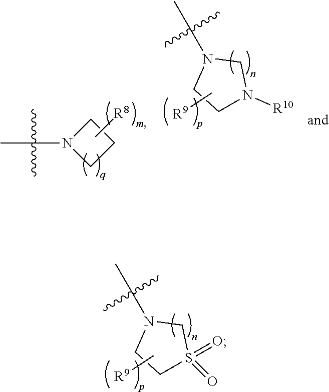

R¹³ is selected from the group consisting of halo, —OR^{f}, and C₁-C₆ alkyl, wherein the C₁-C₆ alkyl is optionally substituted with one or more halo, preferably F;
R^{f} is selected from the group consisting of H and C₁-C₆ alkyl, wherein the C₁-C₆ alkyl is optionally substituted with one or more halo, preferably F;
r is 0, 1, 2, 3, or 4; and
wherein R⁷, R⁸, R⁹, R¹⁰, R^{a}, R^{b}, R^{c}, R^{d}, R^{e}, m, n, p, and q are as defined for the first aspect of the invention, including all preferences etc. thereof.

Compounds of Formulae (IIA) and (IIB) differ in respect of the location of group R¹¹ on the pyrazole. Compounds of both formulae may inhibit PDE10A, however, the compounds of Formula (IIA) are preferred due to the additional advantages that they provide. Therefore, a feature of the second aspect of the invention is that the compound is of Formula (IIA).

It is preferable that R¹¹ is C₁-C₆ alkyl, and more preferably C₁-C₃ alkyl, and even more preferably Me.

It is preferable that R¹² is selected from the group consisting of —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e},

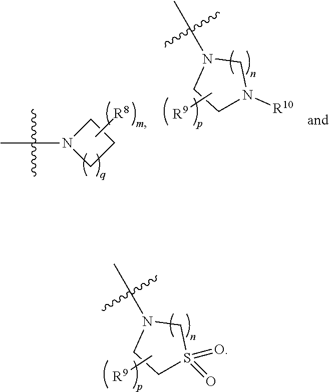

Without wishing to be bound by theory, the presence of a substituent other than hydrogen in the R¹² position may lead to

It is more preferable that R¹² is selected from the group consisting of —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e}, and more preferable —C(O)N═S(O)R^{e}₂. In relation to R¹², it is preferable that R^{e} is Me, cyclopropyl, or two R^{e} groups attached to the same atom can be taken together with the atom to which they are attached to form a 5- or 6-membered heterocycle, most preferably each R^{e} is Me. The most preferable group for R¹² is therefore —C(O)N═S(O)Me₂.

As mentioned, r is 0, 1, 2, 3, or 4, however, it is preferred that r is 0.

In a feature of the second aspect of the invention, R¹¹ is Me;
R¹² is selected from the group consisting of —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e}, and more preferable —C(O)N═S(O)R^{e}₂.
each R^{e} is Me; and
r is 0.

Notwithstanding the above, N-[dimethyl(oxo)-λ6-sulfanylidene]-2-[[4-[1-methyl-4-(4-pyridyl)pyrazol-3-yl]phenoxy]methyl]imidazo[1,2-a]pyridine-3-carboxamide, or a pharmaceutically acceptable salt, solvate, hydrate, tautomer, optical isomer, N-oxide, and/or prodrug thereof, a preferred example of the second aspect of the invention.

In the features of the first and second aspects of the invention mentioned herein, where only certain variables are defined, it is intended that the remainder of the variables are as defined in any other feature herein. Thus, the invention provides for the combination of limited or optional definitions of variables.

As used herein, “optionally substituted” means the group referred to can be unsubstituted, or substituted at one or more positions, i.e. one, two, three, four, five, six or more positions, by any one or any combination of the substituents, such as those listed thereafter.

As used herein, the term “halo” refers to fluoro, chloro, bromo, and iodo. It is preferable that halo is fluoro or chloro, which may be denoted as F and Cl, respectively. It is most preferred that halo is F.

As used herein, the term “(C₁-C₆)alkyl” refers to a fully saturated branched, unbranched or cyclic hydrocarbon moiety having 1, 2, 3, 4, 5 or 6 carbon atoms. It is preferable at each and every instance herein that (C₁-C₆)alkyl is (C₁-C₃)alkyl. That is a fully saturated branched, unbranched or cyclic hydrocarbon moiety having 1, 2 or 3 carbon atoms. Representative examples of alkyl include, but are not limited to, methyl, ethyl, n-propyl, iso-propyl, and cyclopropyl.

The term “oxo” means ═O. It will be understood that an oxo group is divalent and therefore replaces two hydrogen atoms on a single carbon atom when used as a substituent.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

The compounds of the invention may be present as their pharmaceutically acceptable salts. A “pharmaceutically acceptable salt” is intended to mean a salt of a free acid or base of a compound represented by one of the aforementioned Formulae that is non-toxic, biologically tolerable, or otherwise biologically suitable for administration to a subject. Such pharmaceutically acceptable salts are known to those skilled in the art.

Examples of suitable pharmaceutically acceptable salts are those that are pharmacologically effective and suitable for contact with the tissues of subjects without undue toxicity, irritation, or allergic response. A compound of the invention, may possess a sufficiently acidic group, a sufficiently basic group, or both types of functional groups, and accordingly react with a number of inorganic or organic bases, and inorganic and organic acids, to form a pharmaceutically acceptable salt.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate, trifluoroacetate and trifluoromethylsulfonate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoromethylsulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper, particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Examples of pharmaceutically acceptable salts particularly include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

Additionally, any formula given herein is intended to refer also to hydrates and solvates of compounds of the invention, and mixtures thereof, even if such forms are not listed explicitly. A compound of the invention, or pharmaceutically acceptable salt of a compound of the invention, may be obtained as a solvate.

Solvates include those formed from the interaction or complexation of compounds of the invention with one or more solvents, either in solution or as a solid or crystalline form. The solvent may be water, which case the solvates are hydrates.

In addition, certain crystalline forms of a compound of the invention, or a pharmaceutically acceptable salt of a compound of the invention, may be obtained as co-crystals. A compound of the invention, or a pharmaceutically acceptable salt of a compound of the invention, may be obtained in a crystalline form.

A compound of the invention, may be obtained in one of several polymorphic forms, as a mixture of crystalline forms, as a polymorphic form, or as an amorphous form. A compound of the invention may convert in solution between one or more crystalline forms and/or polymorphic forms.

Compounds of the invention that contain groups capable of acting as donors and/or acceptors for hydrogen bonds may be capable of forming co-crystals with suitable co-crystal formers. These co-crystals may be prepared from compounds of the invention by known co-crystal forming procedures. Such procedures include grinding, heating, co-subliming, co-melting, or contacting in solution compounds of the invention with the co-crystal former under crystallization conditions and isolating co-crystals thereby formed. Hence the invention further provides co-crystals comprising a compound of the invention.

Any formula given herein is intended to represent compounds having structures depicted by the structural formula as well as certain variations or forms. In particular, compounds of any formula given herein may have asymmetric centres and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

Included within the scope of the claimed compounds of the present invention are all stereoisomers, geometric isomers and tautomeric forms of the compounds of the invention, including compounds exhibiting more than one type of isomerism, and mixtures of one or more thereof. Also included are acid addition or base addition salts wherein the counter ion is optically active, for example, D-lactate or L-lysine, or racemic, for example, DL-tartrate or DL-arginine.

Where a compound of the invention contains for example, a keto or guanidine group or an aromatic moiety, tautomeric isomerism (‘tautomerism’) can occur. It follows that a single compound may exhibit more than one type of isomerism. Examples of types of potential tautomerisms shown by the compounds of the invention include, amide ⇔ hydroxyl-imine and keto ⇔ enol tautomersims.

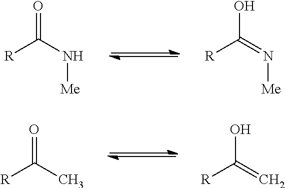

Cis/trans isomers may be separated by conventional techniques well known to those skilled in the art, for example, by chromatography and fractional crystallisation.

Conventional techniques for the preparation/isolation of individual enantiomers include chiral synthesis from a suitable optically pure precursor or resolution of the racemate (or the racemate of a salt or other derivative) using, for example, chiral high pressure liquid chromatography (HPLC).

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% ethanol, typically from 2 to 20%. Concentration of the eluate affords the enriched mixture.

Mixtures of stereoisomers may be separated by conventional techniques known to those skilled in the art.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral centre of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration.

All tautomeric forms are also intended to be included. Tautomers are one of two or more structural isomers that exist in equilibrium and are readily converted from one isomeric form to another. Examples of tautomers include but are not limited to those compounds defined in the claims. Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g. by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions, these less pure preparations of the compounds should contain at least 1%, more suitably at least 5% and preferably from 10 to 59% of a compound of the invention.

When both a basic group and an acid group are present in the same molecule, the compounds of the present invention may also form internal salts, e.g., zwitterionic molecules.

The invention also relates to pharmaceutically acceptable prodrugs of a compound of the invention and treatment methods employing such pharmaceutically acceptable prodrugs.

The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formulae (IA), (IB), (IIA), or (IIB)).

A “pharmaceutically acceptable prodrug” is a prodrug that is non-toxic, biologically tolerable, and otherwise biologically suitable for administration to the subject.

A prodrug is an active or inactive compound that is modified chemically through in vivo physiological action, such as hydrolysis, metabolism and the like, into a compound of the invention following administration of the prodrug to a subject. The compounds of the present invention may themselves be active and/or act as prodrugs which convert in vivo to active compounds. The suitability and techniques involved in making and using pro-drugs are well known by those skilled in the art. Prodrugs can be conceptually divided into two non-exclusive categories, bioprecursor prodrugs and carrier prodrugs. Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity. Carrier prodrugs are drug compounds that contain a transport moiety, e.g., that improve uptake and/or localized delivery to a site(s) of action.

Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde. Moreover, drugs containing an acidic NH group, such as imidazole, imide, indole and the like, have been masked with N-acyloxymethyl groups. Hydroxy groups have been masked as esters and ethers.

The compounds of the invention may also be N-oxides. It will be understood that an N-oxide, or “amine oxide”, is a compound that contains an N—O coordinate covalent bond. Examples of an N-oxide group include the following functional groups.

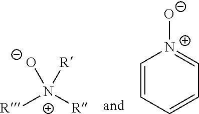

Any formula given herein is also intended to represent unlabelled forms as well as isotopically labelled forms of the compounds. Isotopically labelled compounds have structures depicted by the formulas given herein except that one or more atoms are replaced by an atom having a selected atomic mass or mass number.

Examples of isotopes that can be incorporated into compounds of the invention include isotopes of hydrogen, carbon, nitrogen, oxygen, and fluorine, such as ²H, ³H, ¹¹C, ¹³C, ¹⁴C, ¹³N, ¹⁵N, ¹⁵O, ¹⁷O, ¹⁸O, ¹⁸F, respectively. Such isotopically labelled compounds are useful in metabolic studies (preferably with ¹⁴C), reaction kinetic studies (with, for example ²H or ³H), detection or imaging techniques (such as positron emission tomography (PET) or single-photon emission computed tomography (SPECT)) including drug or substrate tissue distribution assays, or in radioactive treatment of subjects. Substitution with positron emitting isotopes, such as ¹¹C, ¹⁸F, ¹⁵O and ¹³N, can be useful in PET studies for examining substrate receptor occupancy. In particular, an ¹⁸F or ¹¹C labelled compound may be particularly preferred for PET studies. Further, substitution with heavier isotopes such as deuterium (i.e., ²H) may afford certain therapeutic advantages resulting from greater metabolic stability, for example increased in vivo half-life or reduced dosage requirements. Certain isotopically-labelled compounds of the invention for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopically labelled compounds of this invention and prodrugs thereof can generally be prepared by carrying out the procedures disclosed in the schemes or in the examples and preparations described below by substituting a readily available isotopically labelled reagent for a non-isotopically labelled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

If a chemical structure and the associated chemical name do not agree, then the chemical structure takes precedence, unless it is readily understood that the converse is true.

As the compounds of the invention may be used in the prevention and/or treatment of a disease or condition susceptible to PDE10A inhibition, in a third aspect of the invention there is provided a pharmaceutical composition comprising a compound of the invention. As is known, pharmaceutical compositions may comprise one or more excipients in addition to other optional ingredients. It is preferred that the excipients are pharmaceutically acceptable excipients.

The compounds of the invention may be used alone or in combination with one or more additional active ingredients, to formulate pharmaceutical compositions of the invention. A pharmaceutical composition of the invention may comprise (a) an effective amount of at least one compound of the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavouring agents, dyes, and the like and combinations thereof, as would be known to those skilled in the art. Except insofar as any conventional carrier is incompatible with the active ingredient, its use in the therapeutic or pharmaceutical compositions is contemplated.

Pharmaceutical compositions according to the invention may be formulated in conventional manner using readily available ingredients. Thus, the active ingredient may be incorporated, optionally together with other active substances, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, sterile packaged powders, and the like.

The pharmaceutical compositions can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

When pharmaceutical compositions are tablets or gelatin capsules, they may comprise the active ingredient together (compound of the invention) with
a) diluents, e.g., lactose, polylactone, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavouring agents, colouring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period.

For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

The compounds of the invention may be administered topically. Suitable compositions for topical application to the skin or mucosa (e.g., to the skin and eyes), that is dermally or transdermally, include aqueous solutions, suspensions, ointments, creams, gels, hydrogels, microemulsions, dusting powders, dressings, foams, films, skin patches, wafers, implants, fibres, bandages or sprayable formulations, e.g., for delivery by aerosol or the like. Such topical delivery systems will in particular be appropriate for dermal application, e.g., for the treatment of atopic dermatitis. They are thus particularly suited for use in topical, including cosmetic, formulations well-known in the art. Such may contain solubilizers, stabilizers, tonicity enhancing agents, buffers and preservatives. Typical carriers include alcohol, water, mineral oil, liquid petrolatum, white petrolatum, glycerin, polyethylene glycol and propylene glycol. Penetration enhancers may be incorporated.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The present invention further provides anhydrous pharmaceutical compositions and dosage forms comprising compounds of the invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained.

Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compounds of the invention may be administered either simultaneously with, or before or after, one or more other therapeutic agents. The compound of the invention may be administered separately, by the same or different route of administration, or together in the same pharmaceutical composition as the other agents.

The invention includes a product comprising a compound of the invention and at least one other therapeutic agent as a combined preparation for simultaneous, separate or sequential use in therapy. The therapy may be the treatment of a condition or disorder which is mediated by PDE10A. Products provided as a combined preparation include a composition comprising a compound of the invention and the other therapeutic agent(s) together in the same pharmaceutical composition, or the agent of the invention and the other therapeutic agent(s) in separate form, e.g. in the form of a kit.

### Treatments and Methods

The compounds of the invention may prevent and/or treat inflammatory bowel disease, such as ulcerative colitis and/or Crohn's disease. Without wishing to be bound by theory, the treatment may be achieved due to the ability of the compounds of the invention to inhibit PDE10A.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). The terms “treat”, “treating” or “treatment” also refer to alleviating or ameliorating at least one physical parameter including those which may not be discernible by the patient. The treatment may be a physiological treatment (e.g., stabilization of a discernible symptom), a physical treatment (e.g., stabilization of a physical parameter), or both. The terms “treat”, “treating” or “treatment” also refer to preventing or delaying the onset or development or progression of the disease or disorder.

“Prevention” of a condition or disorder refers to delaying or preventing the onset of a condition or disorder or reducing its severity, as assessed by the appearance or extent of one or more symptoms of said condition or disorder.

The fourth aspect of the invention relates to uses of a compound of the invention, or a pharmaceutical composition comprising a compound of the invention.

A compound of the invention, or a pharmaceutical composition comprising a compound of the invention, may be for use as a medicament.

One feature of the fourth aspect of the invention is therefore the use of a compound of the invention for the manufacture of a medicament. The medicament may be for the prevention and/or treatment (preferably the treatment) of inflammatory bowel disease, such as ulcerative colitis and/or Crohn's disease.

The compound of the invention, or a pharmaceutical composition comprising a compound of the invention, may be for use in the prevention and/or treatment (preferably the treatment) of an inflammatory bowel disease, such as ulcerative colitis and/or Crohn's disease.

There is also described herein a method for the prevention and/or treatment of a disease or condition comprising administering to a subject a compound of the invention, or a pharmaceutical composition comprising a compound of the invention, wherein the disease or condition is susceptible to PDE10A inhibition.

The disease or condition susceptible to PDE10A inhibition may be inflammatory bowel disease, such as ulcerative colitis and/or Crohn's disease.

Another method is for the prevention and/or treatment of inflammatory bowel disease comprising administering to a subject a compound of the invention, or a pharmaceutical composition comprising a compound of the invention.

The aforementioned methods are preferably those wherein the inflammatory bowel disease is ulcerative colitis and/or Crohn's disease.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. It is preferable that the subject is a primate, and most preferable that the subject is a human.

Whilst the compound of the invention, and related pharmaceutical compositions, may be used in prevention and/or treatment, it is preferable at every instance that they are for treatment. The methods may therefore be in relation to subject in need of treatment. As used herein, a subject is “in need of” a treatment if such subject would benefit biologically, medically or in quality of life from such treatment.

The compounds of the invention and related pharmaceutical compositions should be provided to subjects in a therapeutically effective amount. The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to at least partially alleviating, inhibiting, preventing and/or ameliorating a condition or disorder which is mediated by PDE10A. In another non-limiting embodiment, the term “a therapeutically effective amount” refers to the amount of the compound of the invention that, when administered to a cell, or a tissue, or a non-cellular biological material, or a medium, is effective to at least partially inhibiting PDE10A activity.

### Preparation of Compounds of the Invention

The compounds of the formulae above may be prepared by, or in analogy with, conventional methods. The preparation of intermediates and compounds according to the examples of the present invention may in particular be illuminated by the following Schemes. Definitions of variables in the structures in schemes herein are commensurate with those of corresponding positions in the formulas delineated herein.

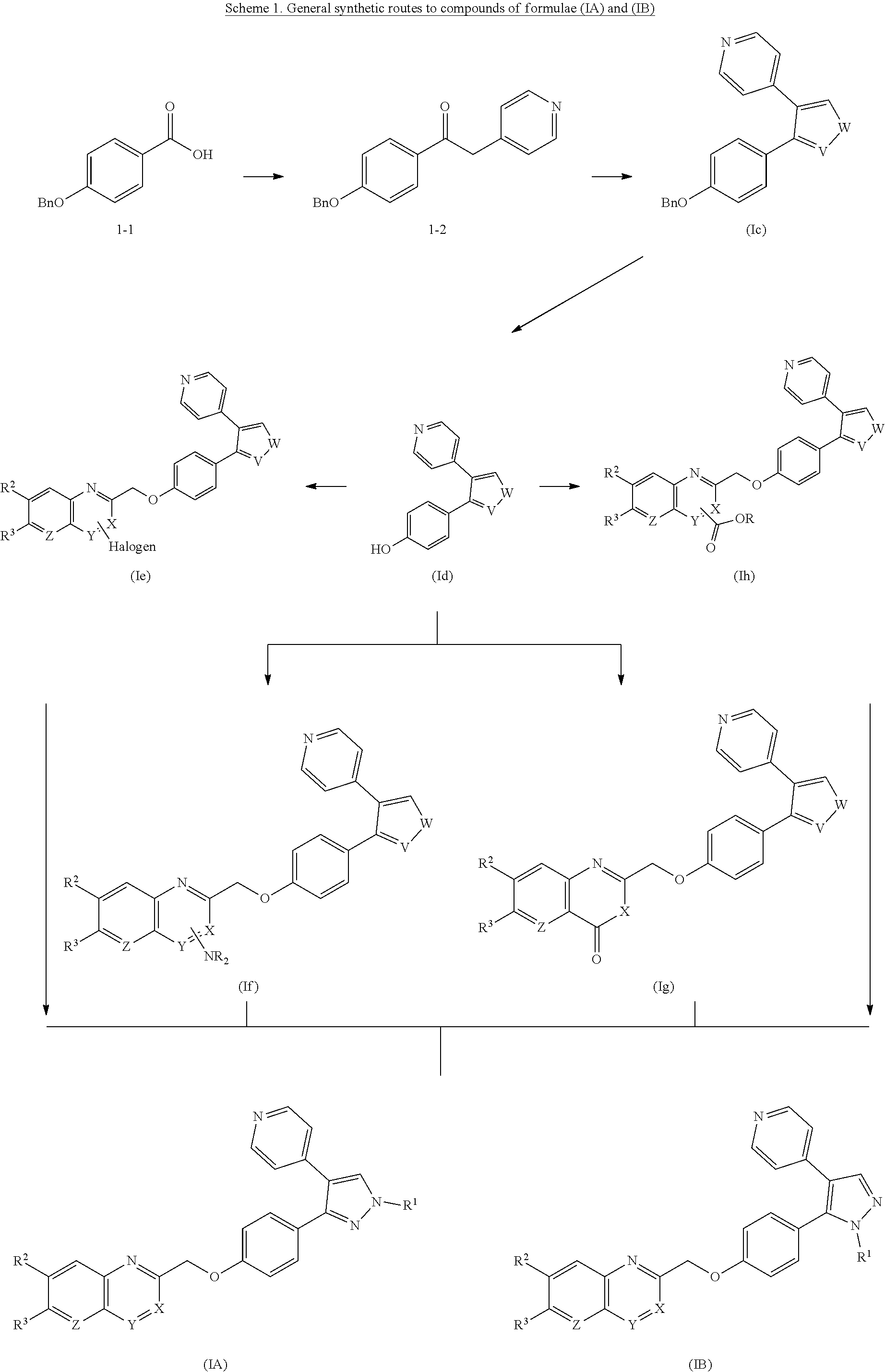

In Scheme 1, V and Ware selected from N and NR¹ as required in formulae (IA) and (IB). Group “—OR” may be O-alkyl, such as —OMe, -OEt etc, group “—NR₂” may be:

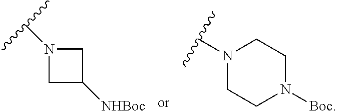 [SMILES: *N1CC(NC(=O)OC(C)(C)C)C1 ; *N1CCN(C(=O)OC(C)(C)C)CC1]

Referring to Scheme 1, compounds of general formulae (IA) and (IB) may be prepared by standard means. For example, 4-benzyloxyphenyl carboxylic acid (1-1) may be converted to the Weinreb amide using amide coupling conditions followed by acylation with 4-methyl pyridine anion to give intermediate (1-2). Intermediate (1-2) may be converted to compounds of general formula (Ic) by reaction with DMFDMA followed by the appropriate hydrazine analogue. Where the pyrazole nitrogen is unsubstituted, this may be alkylated or protected using standard protecting groups such as Boc and SEM. Removal of the benzyl group under hydrogenation conditions gives compounds of general formula (Id). Compounds of general formula (Id) may be reacted with compounds of general formula (Ii) (shown in Scheme 2), or intermediates 3-2, 3-5 and 3-8 (shown in Scheme 3) under alkylation or Mitsunobu coupling conditions to give compounds of general formula (Ie-Ih). Compounds of general formula (Ie) may be converted to compounds of general formulae (IA) and (IB) using standard SNAr reaction conditions with the appropriate nucleophile followed by oxidation and imine formation as required, or using Buchwald coupling conditions, deprotection and urea formation as required. Compounds of general formula (If) may be converted to compounds of general formulae (IA) and (IB) by deprotection as required. Compounds of general formula (Ig) may be converted to compounds of general formulae (IA) and (IB) by reaction with BOP, suitable base and the appropriate amine. Compounds of general formula (Ih) may be converted to compounds of general formulae (IA) and (IB) by introduction of a methyl group by Suzuki coupling where required, saponification, amide coupling and alkylation where required.

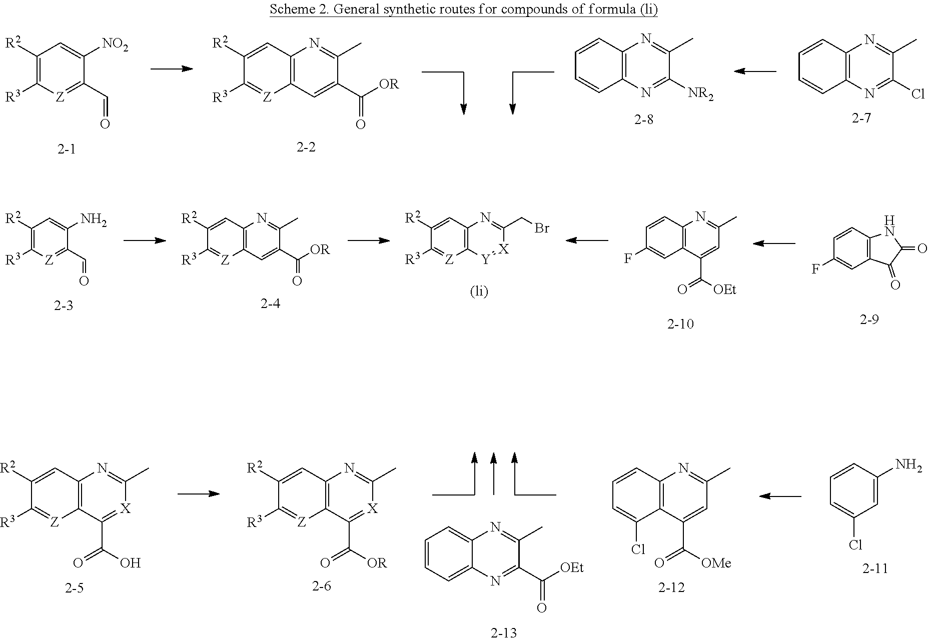

In Scheme 2, group “—OR” may be O-alkyl, such as —OMe, -OEt etc, group “—NR₂” may be:

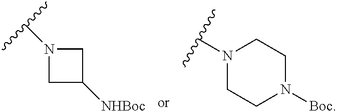 [SMILES: *N1CC(NC(=O)OC(C)(C)C)C1 ; *N1CCN(C(=O)OC(C)(C)C)CC1]

With reference to Scheme 2, compounds of general formula (Ii) may be prepared by standard means. For example, intermediates (2-2) may be prepared by reductive condensation from the appropriate nitro aldehyde and acetoacetates. Intermediates (2-4) may be prepared by condensation from the appropriate amino aldehyde and acetoacetates. Intermediates (2-6) may be prepared by esterification of the carboxylic acids. Intermediates (2-8) may be prepared by SNAr of the aryl chloride with the appropriate amine. Intermediates (2-10) may be prepared by ring expansion of the dicarbonyl compound followed by esterification. Intermediates (2-12) may be prepared by condensation of chloroanilines with acetoacetates followed by bromination, carbonylation and esterification. Bromination of intermediates (2-2), (2-4), (2-6), (2-8), (2-10), (2-12) and (2-13) using NBS may yield compounds of general formula (Ii).

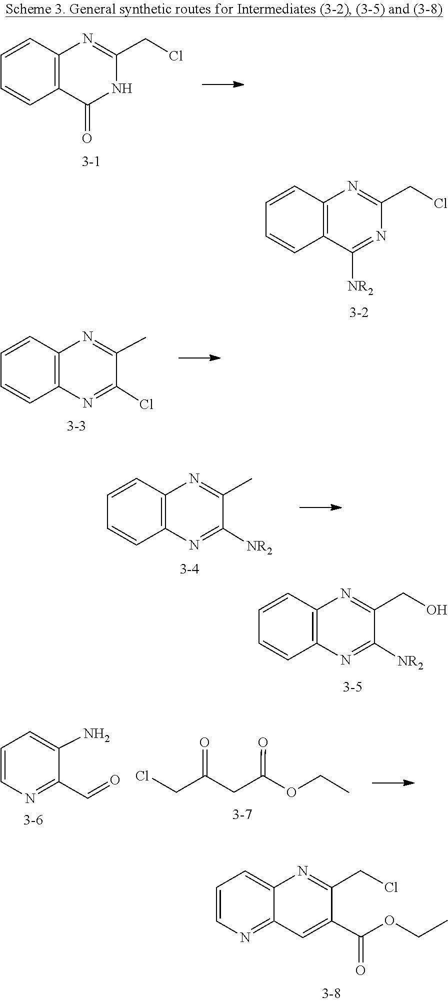

In Scheme 3, group “—NR₂” may be:

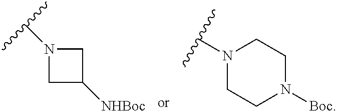 [SMILES: *N1CC(NC(=O)OC(C)(C)C)C1 ; *N1CCN(C(=O)OC(C)(C)C)CC1]

In accordance with Scheme 3, intermediates (3-2), (3-5) and (3-8) may be prepared by standard means. For example, 2-(chloromethyl)-3H-quinazolin-4-one (3-1) may be treated with POCl₃ to generate an aryl chloride which may be subjected to standard SNAr conditions with the appropriate amine to give intermediates (3-2). 2-Chloro-3-methylquinoxaline (3-3) may undergo SNAr with the appropriate amine followed by oxidation and reduction to give alcohol intermediates (3-5). Condensation of 3-aminopicolinaldehyde (3-6) and ethyl 4-chloro-3-oxobutanoate (3-7) may give intermediate (3-8).

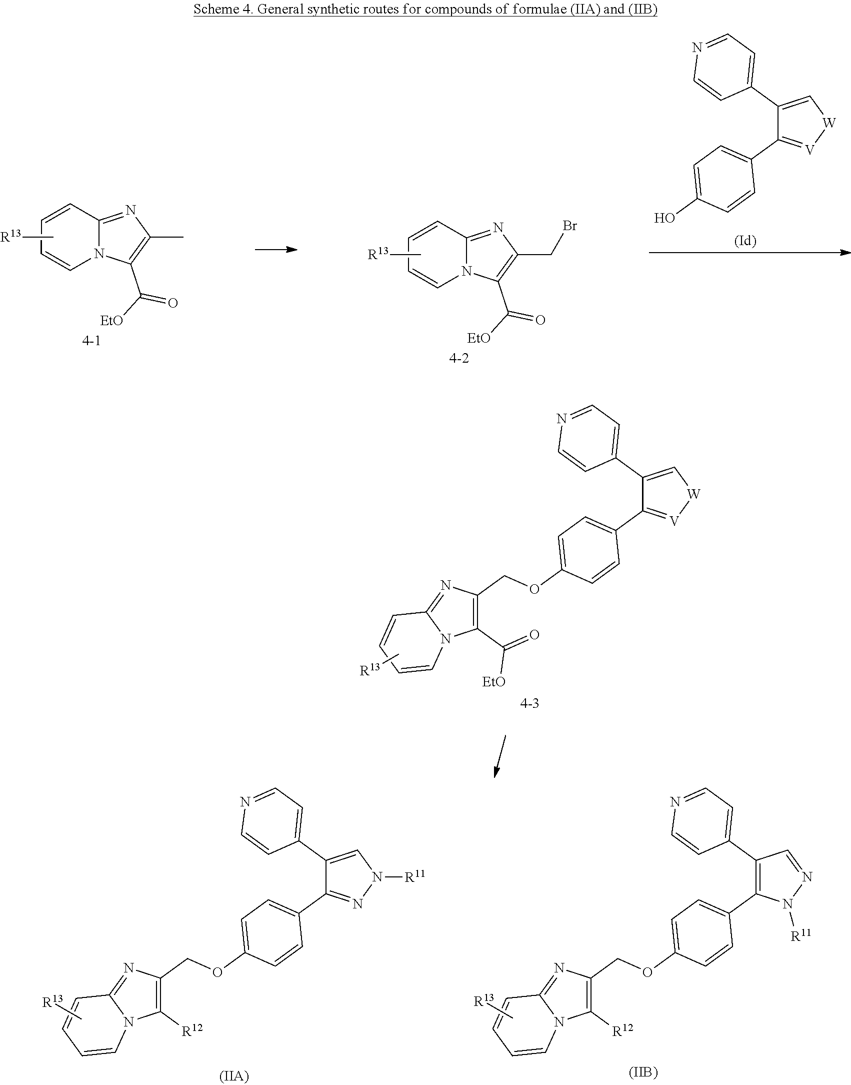

With reference to Scheme 4, compounds of general formulae (IIA) and (IIB) may be prepared by standard means. For example, intermediates (4-1) may be brominated using NBS followed by alkylation with compounds of general formula (Id) to give intermediates (4-3). Saponification followed by amide coupling with the appropriate amines using standard amide coupling conditions such as HATU gives compounds of general formulae (IIA) and (IIB).

### EXAMPLES

Exemplary compounds of the invention, and exemplary compounds useful in methods of the invention, will now be described by reference to the illustrative synthetic schemes for their general preparation below and the specific examples that follow. Artisans will recognise that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Reactions may be performed between the melting point and the reflux temperature of the solvent, or at higher temperatures by using seal reaction vessels, and preferably between 0° C. and the reflux temperature of the solvent. Reactions may be heated employing conventional heating or microwave heating. Reactions may also be conducted in sealed pressure vessels above the normal reflux temperature of the solvent.

All of the derivatives of the compounds of the invention, and in particular the above-mentioned Formulae, can be prepared by the procedures described in the general methods presented below or by routine modifications thereof. The present invention also encompasses any one or more of these processes for preparing the derivatives of the Formulae, in addition to any novel intermediates used therein.

The routes below, including those mentioned in the Examples and Intermediates, illustrate methods of synthesising the compounds of the invention. The skilled person will appreciate that the compound of the invention, and intermediates thereto, could be made by methods other than those specifically described herein, for example by adaptation of the methods described herein, for example by methods known in the art.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect phenol or carboxylic acid groups. The protecting groups used in the preparation of the compounds of the invention may be used in a conventional manner.

In the general synthetic methods below, unless otherwise specified, the substituents are as defined above with reference to the compound of the various formulae above.

Where ratios of solvents are given, the ratios are by volume.

The skilled person will appreciate that the experimental conditions set forth in the schemes that follow are illustrative of suitable conditions for effecting the transformations shown, and that it may be necessary or desirable to vary the precise conditions employed for the preparation of the compound of the invention. It will be further appreciated that it may be necessary or desirable to carry out the transformations in a different order from that described in the schemes, or to modify one or more of the transformations, to provide the desired compound of the invention.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The compounds of the invention may be prepared by any method known in the art for the preparation of compounds of analogous structure. In particular, the compound of the invention can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of the compound of the invention. It will be further appreciated that it may be necessary or desirable to carry out the transformations in a different order from that described in the schemes, or to modify one or more of the transformations, to provide the desired compound of the invention

The following abbreviations have been used:
aq aqueous
Boc tert-butyloxycarbonyl
Bn benzyl
BOP benzotriazol-1-yloxytris(dimethylamino)phosphonium hexafluorophosphate
DCM dichloromethane
DIPEA diisopropylethylamine
DMAP 4-dimethylaminopyridine
DMF dimethylformamide
DMFDMA N,N-dimethylformamide dimethyl acetal
dppf 1,1′-Bis(diphenylphosphino)ferrocene
ES+ electrospray ionization
h hour(s)
HATU (1-[bis(dimethylamino)methylene]-1H-1,2,3-triazolo[4,5 b]pyridinium 3-oxide hexafluorophosphate
HPBC hydroxypropyl-β-cyclodextrin
HPLC high performance liquid chromatography
LCMS liquid chromatography-mass spectrometry
min minute(s)
NBS N-bromosuccinimide
NMP N-methyl-2-pyrrolidone
Rt retention time
sat saturated
TEA triethylamine
TFA trifluoroacetic acid
THF tetrahydrofuran
TLC thin layer chromatography
UPLC ultra performance liquid chromatography

### Experimental Methods

Reactions were conducted at room temperature unless otherwise specified. Microwave reactions were performed with a Biotage microwave reactor using process vials fitted with aluminium caps and septa. Preparative chromatography was performed using CombiFlash systems equipped with Isolute Flash II silica columns. Reverse phase column chromatography was performed using CombiFlash systems equipped with RediSep Rf C18 columns. Reverse Phase HPLC was performed on either a Gilson system with a UV detector or an ACCQPrep system with UV and mass detection, equipped with ACE-5AQ, 100×21.2 mm, 5 μm columns. The purest fractions were collected, concentrated, and dried under vacuum. Compounds were typically dried in a vacuum oven at 50-60° C. prior to purity analysis. Compound analysis was performed by UPLC using an Agilent 1290 Infinity system (Methods listed below). LCMS analysis was performed using a Waters UPLC Acquity H-Class system with PDA and QDa detectors or Agilent 6140 Series Quadrupole Mass Spectrometer with a multimode source using Phenomenex Kinetex XB-C18 columns, or a Shimadzu LCMS-2020 system with PDA: SPD-M40 and MS: LCMS-2020 detectors using Kinetex EVO C18 columns. HRMS analysis was performed using a Waters UPLC Acquity H-Class/XevoG2 QToF system with Acquity PDA detector using Waters HSS T3 columns. The compounds prepared were named using IUPAC nomenclature. All yields quoted factor in purity of the product except those indicated with an asterix.

### UPLC Methods

Method A (10 min, 5-100)

Phenomenex Kinetex XB-C18, 1.7 μm, 2.1×100 mm, 40° C., 0.5 mL/min, 5% MeCN (+0.085% TFA) in water (+0.1% TFA) for 1.0 min, 5-100% over 8.0 min, hold for 0.2 min, re-equilibrate 0.8 min. 200-300 nm.

Method B (5 min, 5-100)

Phenomenex Kinetex XB-C18, 1.7 μm, 2.1×50 mm, 40° C., 0.8 mL/min, 5% MeCN (+0.1% TFA) in water (+0.1% TFA) for 1.0 min, 5-100% over 3.0 min, hold for 0.2 min, re-equilibrate 0.8 min. 200-300 nm.

Method C (5 min, 5-100)

Phenomenex Kinetex XB-C18, 1.7 μm, 2.1×50 mm, 40° C., 0.8 mL/min, 5% MeCN (+0.1% formic acid) in water (+0.1% formic acid) for 0.7 min, 5-100% over 3.0 min, hold for 0.3 min, re-equilibrate 1.0 min. 254 nm.

### INTERMEDIATE 1

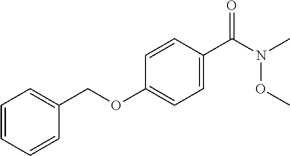 [SMILES: CON(C)C(=O)c1ccc(OCc2ccccc2)cc1]

### 4-Benzyloxy-N-methoxy-N-methyl-benzamide

Oxalyl chloride (3.76 mL, 43.8 mmol) was added dropwise to a suspension of 4-benzyloxybenzoic acid (5.00 g, 21.9 mmol) in DCM (75 mL) and DMF (400 μL) at 0° C. The reaction was warmed to room temperature, stirred for 2 h then concentrated in vacuo. The residue was dissolved in DCM (100 mL) and N,O-dimethylhydroxylamine hydrochloride (2.14 g, 21.9 mmol) was added. The reaction was cooled to 0° C. and TEA (7.63 mL, 54.8 mmol) was added dropwise then warmed to room temperature and stirred for 18 h. The reaction mixture was partitioned between DCM (250 mL) and sat aq NaHCO₃ (250 mL). The aq layer was extracted with DCM (250 mL) and the organic layers combined, washed with brine (250 mL), dried (MgSO₄) and concentrated in vacuo. The residue was purified by normal phase column chromatography to give the title compound (4.53 g, 76.3%) as a white solid. UPLC (Method A) Rt 5.53 min, 100%. LCMS (ES+): 272.1 [MH]⁺.

### INTERMEDIATE 2

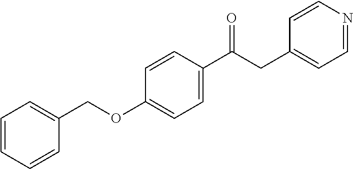 [SMILES: O=C(Cc1ccncc1)c1ccc(OCc2ccccc2)cc1]

### 1-(4-Benzyloxyphenyl)-2-(4-pyridyl)ethanone

Under N₂ n-BuLi (2.5M in hexanes, 13.4 mL, 33.4 mmol) was added dropwise to a solution of diisopropylamine (4.71 mL, 33.4 mmol) in THF (40 mL) at −78° C. The reaction was stirred for 30 min, warmed to 0° C. and stirred for 30 min. 4-Methylpyridine (3.28 mL, 33.4 mmol) was added dropwise and the reaction was stirred for 30 min. In a separate flask under N₂, intermediate 1 (4.53 g, 16.7 mmol) was dissolved in THF (100 mL) and cooled to −78° C. The solution of 4-methylpyrine anion was added dropwise over 1 h. The reaction was stirred for 1 h. AcOH (20 mL) was added, and the reaction was allowed to warm to room temperature overnight. The reaction mixture was concentrated in vacuo then partitioned between DCM (250 mL) and water (250 mL). The aq portion was extracted with DCM (250 mL). The combined organic portions were washed with sat NaHCO₃ (250 mL), dried (MgSO₄) and concentrated in vacuo to give the title compound (4.85 g, 93.0%) as a light yellow solid. UPLC (Method A) Rt 4.67 min, 97.2%. LCMS (ES+): 304.2 [MH]⁺.

### INTERMEDIATES 3 AND 4

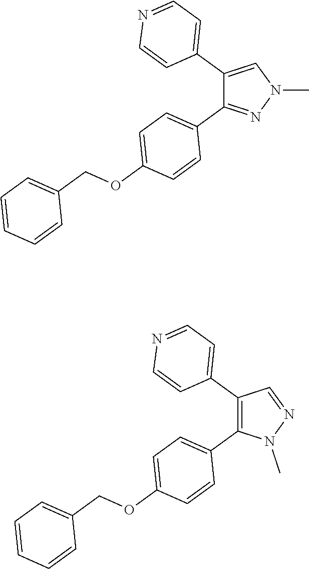 [SMILES: Cn1cc(-c2ccncc2)c(-c2ccc(OCc3ccccc3)cc2)n1 ; Cn1ncc(-c2ccncc2)c1-c1ccc(OCc2ccccc2)cc1]

### 4-[3-(4-Benzyloxyphenyl)-1-methyl-pyrazol-4-yl]pyridine and 4-[5-(4-benzyloxyphenyl)-1-methyl-pyrazol-4-yl]pyridine

Intermediate 2 (3.85 g, 97.2% pure, 12.3 mmol) in DMFDMA (25 mL) was heated at reflux for 2 h then concentrated in vacuo. The residue was dissolved in EtOH (60 mL), methylhydrazine (1.95 mL, 37.0 mmol) and conc sulfuric acid (138.3 μL, 2.46 mmol) were added and the reaction was heated at 70° C. for 3 h. The reaction mixture was concentrated in vacuo then partitioned between DCM (250 mL) and sat aq NaHCO₃ (250 mL). The aq layer was extracted with DCM (250 mL) and the organic layers combined, dried (MgSO₄) and concentrated in vacuo. The residue was purified by normal phase column chromatography (1% TEA buffered) to give the title compounds (2.84 g, 65.9%) as a yellow solid and (625 mg, 10.7%) as a yellow solid respectively. UPLC Rt 4.67 min, 97.6%. LCMS (ES+): 342.2 [MH]⁺. UPLC (Method A) 4.74 min, 72.2%. LCMS (ES+): 342.3 [MH]⁺.

### INTERMEDIATE 5

### 4-[3-(4-Benzyloxyphenyl)-1H-pyrazol-4-yl]pyridine

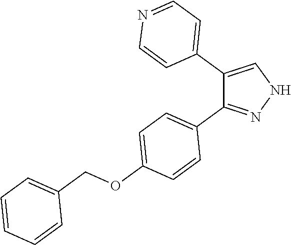 [SMILES: c1ccc(COc2ccc(-c3n[nH]cc3-c3ccncc3)cc2)cc1]

A mixture of intermediate 2 (8.88 g, 97.2% pure, 28.5 mmol) in DMFDMA (56.7 mL, 427 mmol) was heated at reflux for 1.5 h. The reaction mixture was concentrated in vacuo then dissolved in EtOH (250 mL), hydrazine monohydrate (4.23 mL, 85.4 mmol) was added and the reaction was heated at 70° C. for 2 h. The reaction mixture was concentrated in vacuo then triturated with Et₂O (3×100 mL) to give the title compound (8.42 g, 89.7%) as a yellow solid. UPLC (Method A) Rt 4.28 min, 99.2%. LCMS (ES+): ES+: 328.2 [MH]⁺.

### INTERMEDIATE 6

### tert-Butyl 3-(4-benzyloxyphenyl)-4-(4-pyridyl)pyrazole-1-carboxylate

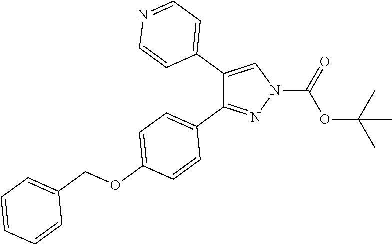 [SMILES: CC(C)(C)OC(=O)n1cc(-c2ccncc2)c(-c2ccc(OCc3ccccc3)cc2)n1]

TEA (950 μL, 6.82 mmol) was added to a solution of intermediate 5 (1.49 g, 4.55 mmol), DMAP (55.5 mg, 455 μmol) and Boc₂O (1.49 g, 6.82 mmol) in THF (45 mL) and the reaction was stirred for 2 h. The reaction mixture was concentrated in vacuo then purified by normal phase column chromatography to give the title compound (1.68 g, 85.9%) as a white solid. UPLC (Method A) Rt 5.58 min, 99.2%. LCMS (ES+): 428.3 [MH]⁺.

### INTERMEDIATE 7

### 2-[[3-(4-Benzyloxyphenyl)-4-(4-pyridyl)pyrazol-1-yl]methoxy]ethyl-trimethyl-silane

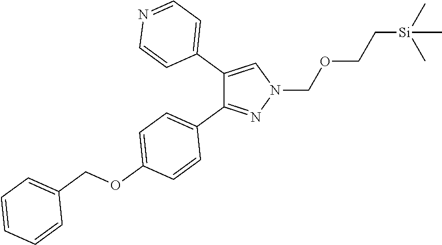 [SMILES: C[Si](C)(C)CCOCn1cc(-c2ccncc2)c(-c2ccc(OCc3ccccc3)cc2)n1]

To a stirred mixture of intermediate 5 (500 mg, 1.53 mmol) and Cs₂CO₃ (1.50 g, 4.58 mmol) in DMF (20 mL) were added SEMCI (306 mg, 1.83 mmol) dropwise at 0° C. under N₂ and stirred for 2 days. The reaction was quenched with water, extracted with EtOAc (3×15 mL), the combined organic layers were washed with water (3×10 mL), dried (Na₂SO₄) then concentrated in vacuo. The residue was purified by Prep-TLC to give the title compound (220 mg, 31.5%)* as an off-white solid. LCMS (ES+): 458.0 [MH]⁺.

### INTERMEDIATE 8

### 4-[3-(4-Benzyloxyphenyl)-1-ethyl-pyrazol-4-yl]pyridine

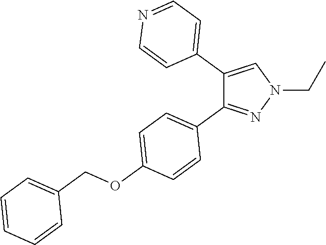 [SMILES: CCn1cc(-c2ccncc2)c(-c2ccc(OCc3ccccc3)cc2)n1]

To a solution of intermediate 5 (700 mg, 2.14 mmol) in dry DMF (15 mL) was added NaH (60% in oil, 102 mg) at 0° C. under N₂. The mixture was stirred for 35 min then ethyl iodide (500 mg, 3.21 mmol) was added and the allowed to warm to room temperature for 1 h. The reaction was quenched with water, extracted with DCM (3×25 mL), dried (Na₂SO₄) and concentrated in vacuo. The residue was purified by silica gel column chromatography to give the title compound (460 mg, 60.5%)* as an off-white solid. LCMS (ES+): 356.2 [MH]⁺.

### INTERMEDIATES 9 and 10

Intermediates 9 and 10 were prepared similarly to intermediate 8, by coupling of intermediate 5 with the appropriate alkylhalide; see Table 1 below.

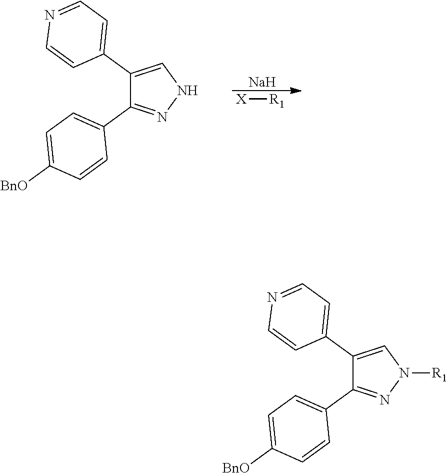

X-R₁ is Br—R₁ or I—R₁.

TABLE 1
Alkylation of pyrazoles

| | | | Halide used, Form, |
|---|---|---|---|
| Int | Structure | Name | Yield, LCMS |
| 9 | 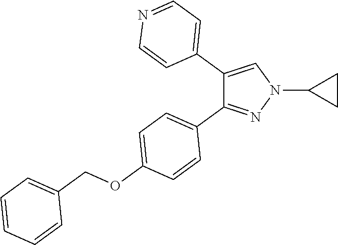 [SMILES: c1ccc(COc2ccc(-c3nn(C4CC4)cc3-c3ccncc3)cc2)cc1] | 4-[3-(4- Benzyloxyphenyl)-1- cyclopropyl-pyrazol- 4-yl]pyridine | From bromocyclopropane Yellow oil Yield 145 mg, 12.9%* LCMS (ES+): 368.0 [MH⁺ |
| 10 | 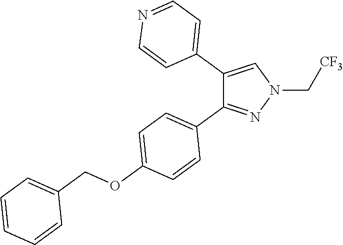 [SMILES: FC(F)(F)Cn1cc(-c2ccncc2)c(-c2ccc(OCc3ccccc3)cc2)n1] | 4-[3-(4- Benzyloxyphenyl)-1- (2,2,2-trifluoroethyl) pyrazol-4-yl]pyridine | From 1,1,1-trifluoro-2- iodoethane Brown solid Yield 112 mg, 17.6%* LCMS (ES+): 410.1 [MH]⁺. |

### INTERMEDIATE 11

### 4-[1-Methyl-4-(4-pyridyl)pyrazol-3-yl]phenol

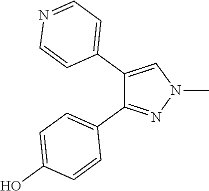 [SMILES: Cn1cc(-c2ccncc2)c(-c2ccc(O)cc2)n1]

Intermediate 3 (3.48 g, 97.6% pure, 9.95 mmol) was dissolved in EtOH (100 mL) and EtOAc (100 mL) and the solution was passed through an H-cube (70×4 mm 10% Pd/C CatCart, 1.0 mL/min, 60° C., 50 bar) twice. The reaction mixture was concentrated in vacuo to give the title compound (2.57 g, 98.9%) as a white solid. UPLC (Method A) Rt 2.73 min, 96.1%. LCMS (ES+): 252.1 [MH]⁺.

### INTERMEDIATE 12

### 4-[2-Methyl-4-(4-pyridyl)pyrazol-3-yl]phenol

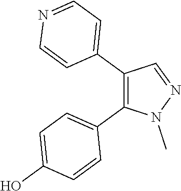 [SMILES: Cn1ncc(-c2ccncc2)c1-c1ccc(O)cc1]

Intermediate 4 (130 mg, 0.38 mmol) was dissolved in MeCOH (15 mL) and passed through an H-cube (70×4 mm 10% Pd/C CatCart, 1.0 mL/min, 35° C.). The reaction was concentrated in vacuo to give the title compound (70.0 mg, 69.9%) as a white solid. UPLC (Method B) Rt 1.68 min, 95.5%. LCMS (ES+): 252.1 [MH]⁺.

### INTERMEDIATE 13

### tert-Butyl 3-(4-hydroxyphenyl)-4-(4-pyridyl)pyrazole-1-carboxylate

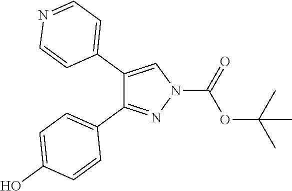 [SMILES: CC(C)(C)OC(=O)n1cc(-c2ccncc2)c(-c2ccc(O)cc2)n1]

Intermediate 6 (1.68 mg, 99.2% pure, 3.91 mmol) was dissolved in EtOH (80 mL) and the solution was passed through an H-cube (30×4 mm 10% Pd/C CatCart, 1.0 mL/min, 22° C., full H₂ mode) in a continuous loop for 6 h. The reaction mixture was concentrated in vacuo to give the tile compound (1.36 g, 99.3%) as a white solid. UPLC (Method A) Rt 3.99 min, 96.2%. LCMS (ES+): 338.2 [MH]⁺.

### INTERMEDIATE 14

### 4-[4-(4-Pyridyl)-1-(2-trimethylsilylethoxymethyl)pyrazol-3-yl]phenol

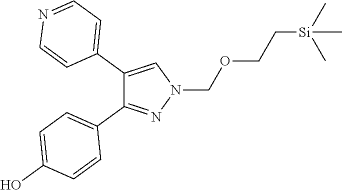 [SMILES: C[Si](C)(C)CCOCn1cc(-c2ccncc2)c(-c2ccc(O)cc2)n1]

To a solution of intermediate 7 (220 mg, 0.48 mmol) in MeOH (10 mL) was added Pd/C (10%, 200 mg) in a pressure vessel. The mixture was hydrogenated under 10 bar of hydrogen for 2.5 h, filtered through a Celite pad and concentrated in vacuo to give the title compound. The crude material was use in the next step without any further purification. LCMS (ES+): 368.1 [MH]⁺.

### INTERMEDIATES 15 to 17

Intermediates 15-17 were prepared similarly to intermediate 14, by deprotection of the benzyl group via hydrogenation, see Table 2 below.

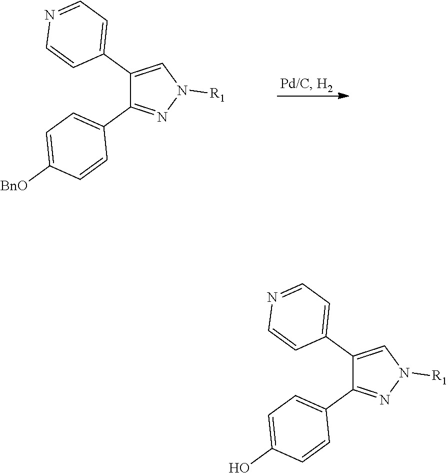

TABLE 2
Deprotection of benzyl group via hydrogenation

| | | | Intermediate, Form, |
|---|---|---|---|
| Int | Structure | Name | Yield, LCMS |
| 15 | 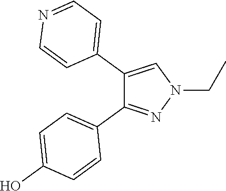 [SMILES: CCn1cc(-c2ccncc2)c(-c2ccc(O)cc2)n1] | 4-[1-Ethyl-4-(4- pyridyl)pyrazol-3- yl]phenol | From intermediate 8 Yellow solid Yield 180 mg, 71.3%* LCMS (ES+): 266.1 [MH]+. |
| 16 | 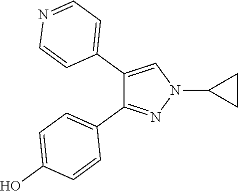 [SMILES: Oc1ccc(-c2nn(C3CC3)cc2-c2ccncc2)cc1] | 4-[1-Cyclopropyl-4- (4-pyridyl)pyrazol-3- yl]phenol | From intermediate 9 Orange solid Yield 56.2 mg, 51.3%* LCMS (ES+): 278.2 [MH]⁺ |
| 17 | 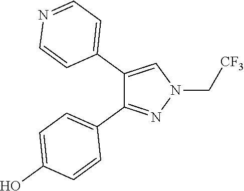 [SMILES: Oc1ccc(-c2nn(CC(F)(F)F)cc2-c2ccncc2)cc1] | 4-[4-(4-Pyridyl)-1- (2,2,2-trifluoroethyl) pyrazol-3-yl]phenol | From intermediate 10 White solid Yield 63.0 mg, 63.5%* LCMS (ES+): 320.1 [MH]⁺ |

### INTERMEDIATE 18

### tert-Butyl 2-methylquinoline-4-carboxylate

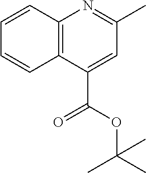 [SMILES: Cc1cc(C(=O)OC(C)(C)C)c2ccccc2n1]

N,N′-Dicyclohexylcarbodiimide (1.65 g, 8.01 mmol) was added portion wise to a suspension of 2-methylquinoline-4-carboxylic acid (1.00 g, 5.34 mmol), DMAP (65.3 mg, 534 μmol) and tert-butanol (1.02 mL, 10.7 mmol) in DCM (60 mL) at the mixture stirred for 16 h. The reaction mixture was filtered and concentrated in vacuo. The residue was purified by normal phase column chromatography to give the title compound (539 mg, 41.2%) as a yellow oil. UPLC (Method A) Rt 4.31 min, 99.4%. LCMS (ES+): 244.2 [MH]⁺.

### INTERMEDIATE 19

### Ethyl 7-fluoro-2-methyl-quinoline-3-carboxylate

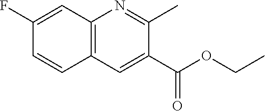 [SMILES: CCOC(=O)c1cc2ccc(F)cc2nc1C]

To a stirred mixture of 4-fluoro-2-nitrobenzaldehyde (2.00 g, 11.8 mmol) and Fe (3.30 g, 59.1 mmol) in AcOH (20 mL) was added ethyl acetoacetate (1.85 g, 14.2 mmol) and stirred at 50° C. for 2 h. The resulting mixture was filtered, the filter cake was washed with DCM (3×30 mL), the combined organic layers were washed with water (3×30 mL), dried (Na₂SO₄) and concentrated in vacuo. The residue was purified by silica gel column chromatography to give the title compound (760 mg, 27.6%)* as a white solid. LCMS (ES+): 234.1 [MH]⁺.

### INTERMEDIATES 20 and 21

Intermediates 20 and 21 were prepared similarly to intermediate 19, by condensation of the appropriate benzaldehyde with ethylacetoacetate, see Table 3 below.

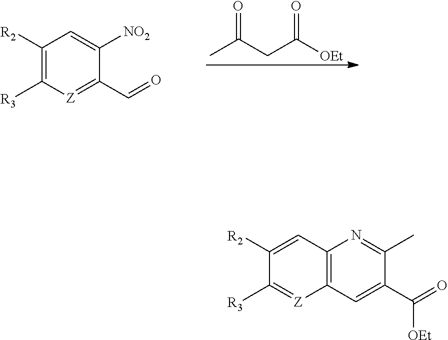

TABLE 3
Condensation reactions to give quinolines

| | | | Intermediate, Form, |
|---|---|---|---|
| Int | Structure | Name | Yield, LCMS |
| 20 | 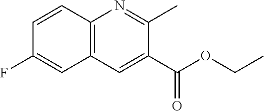 [SMILES: CCOC(=O)c1cc2cc(F)ccc2nc1C] | Ethyl 6-fluoro-2- methyl-quinoline-3- carboxylate | From 5-fluoro-2- nitrobenzaldehyde Yellow solid Yield 670 mg, 24.3%* LCMS (ES+): 234.1 [MH]⁺ |
| 21 | 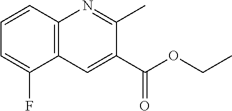 [SMILES: CCOC(=O)c1cc2c(F)cccc2nc1C] | Ethyl 5-fluoro-2- methyl-quinoline-3- carboxylate | From 2-fluoro-6- nitrobenzaldehyde Yellow solid Yield 1.98 g, 71.8%* LCMS (ES+): 234.1 [MH]⁺ |

### INTERMEDIATE 22

### Methyl 6-bromo-2-methyl-quinoline-3-carboxylate

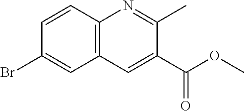 [SMILES: COC(=O)c1cc2cc(Br)ccc2nc1C]

To a stirred solution of 2-amino-5-bromobenzaldehyde (1.70 g, 8.50 mmol) in methyl acetoacetate (10 mL) was added water (30 μL) dropwise then heated at 80° C. for 3 h. EtOAc (50 mL) was added, washed with H₂O (3×20 mL) then concentrated in vacuo. The residue was purified by silica gel column chromatography to give the title compound (1.80 g, 75.6%)* as a yellow solid. LCMS (ES+): 280.0 [MH]⁺.

### INTERMEDIATE 23

### Methyl 5-bromo-2-methyl-quinoline-3-carboxylate

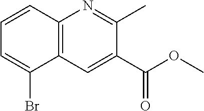 [SMILES: COC(=O)c1cc2c(Br)cccc2nc1C]

A mixture of 2-amino-6-bromobenzaldehyde (1.00 g, 5.00 mmol) and methyl acetoacetate (6.0 mL) and H₂O (0.1 mL) was stirred at 80° C. for 3 h. The resulting mixture was concentrated in vacuo then purified by silica gel column chromatography to give the title compound (0.90 g, 64.3%)* as a yellow solid. LCMS (ES+): 280.2 [MH]⁺.

### INTERMEDIATE 24

### Ethyl 6-fluoro-2-methyl-quinoline-4-carboxylate

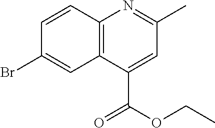 [SMILES: CCOC(=O)c1cc(C)nc2ccc(Br)cc12]

To a stirred mixture of 5-fluoro-1H-indole-2,3-dione (2.00 g, 12.1 mmol) and KOH (3.40 g, 60.6 mmol) in EtOH (165 mL) was added acetone (1.41 g, 24.2 mmol) dropwise then heated at 80° C. for 2 h. The reaction was neutralized to pH 7 with conc. HCl then concentrated in vacuo. The residue was dissolved in EtOH:Toluene (1:1), H₂SO₄ (2.0 mL) was added then heated at 80° C. overnight. The reaction was neutralized to pH 7 with NaOH, extracted with EtOAc (3×50 mL), the combined organic layers washed with water (3×20 mL), dried (Na₂SO₄) and concentrated in vacuo. The residue was purified by silica gel column chromatography to give the title compound (720 mg, 25.5%)* as an off-white solid. LCMS (ES+): 233.9 [MH]⁺.

### INTERMEDIATE 25

### 4-Bromo-5-chloro-2-methyl-quinoline

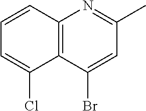

To a stirred mixture of 3-chloroaniline (1.85 g, 14.5 mmol) and ethyl acetoacetate (1.89 g, 14.5 mmol) in dioxane (40 mL) was added polyphosphoric acid (10.0 g, 86.9 mmol) and the reaction heated at 100° C. overnight. The mixture was basified to pH 14 with aq NaOH then extracted with EtOAc (3×30 mL). The aqueous phase was acidified to pH 5 with aq HCl, extracted with EtOAc (3×30 mL). The combined organic layers were washed with water (3×20 mL), dried (Na₂SO₄) and concentrated in vacuo. The residue (600 mg, 3.10 mmol) was dissolved in MeCN (20 mL) and PBr₃ (2.10 g, 7.75 mmol) was added then heated at 80° C. for 3 h. The mixture was basified to pH 9 with sat aq NaHCO₃, extracted with EtOAc (3×15 mL), the combined organic layers were washed with water (3×10 mL), dried (Na₂SO₄) then concentrated in vacuo. The residue was purified by Prep-TLC to give the title compound (310 mg, 39.0%)* as a yellow solid. LCMS (ES+): 258.6 [MH]⁺.

### INTERMEDIATE 26

### Methyl 5-chloro-2-methyl-quinoline-4-carboxylate

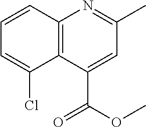 [SMILES: COC(=O)c1cc(C)nc2cccc(Cl)c12]

To a solution of intermediate 25 (290 mg, 1.13 mmol) and TEA (172 mg, 1.70 mmol) in MeCOH (10 mL) was added Pd(dppf)Cl₂ (92.1 mg, 0.11 mmol) in a pressure vessel. The mixture was purged with N₂ for 5 min and then was pressurized to 10 bar with carbon monoxide for 6 h. The resulting mixture was filtered, the filter cake was washed with MeCOH (3×5 mL) then concentrated in vacuo. The residue was purified by silica gel column chromatography to give the title compound (170 mg, 63.8%)* as a yellow oil. LCMS (ES+): 236.0 [MH]⁺.

### INTERMEDIATE 27

### tert-Butyl 4-(3-methylquinoxalin-2-yl)piperazine-1-carboxylate

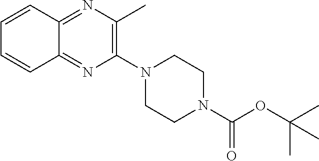 [SMILES: Cc1nc2ccccc2nc1N1CCN(C(=O)OC(C)(C)C)CC1]

To a stirred solution of 2-chloro-3-methylquinoxaline (200 mg, 1.12 mmol) and tert-butyl piperazine-1-carboxylate (209 mg, 1.12 mmol) in DMF was added DIPEA (289 mg, 2.24 mmol) dropwise and stirred 80° C. for 3 h. The reaction was quenched with water, extracted with EtOAc (2×40 mL) then concentrated in vacuo. The residue was purified by Prep-TLC to give the title compound (140 mg, 38.1%) as a light yellow solid. LCMS (ES+): 329.2 [MH]⁺.

### INTERMEDIATE 28

### Methyl 2-(bromomethyl)quinoline-3-carboxylate

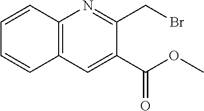 [SMILES: COC(=O)c1cc2ccccc2nc1CBr]

Azobisisobutyronitrile (44.1 mg, 269 μmol) was added to a solution of methyl 2-methylquinoline-3-carboxylate (548 mg, 98.8% pure, 2.69 mmol) and NBS (718 mg, 4.03 mmol) in CCl₄ (13 mL) and the reaction heated at reflux for 4 h. The reaction mixture was filtered and concentrated in vacuo then purified by normal phase column chromatography to give the title compound (492 mg, 60.9%) as a yellow solid. UPLC (Method A) Rt 5.55 min, 93.2%. LCMS (ES+): 280.0 [MH]⁺.

### INTERMEDIATES 29 to 32

Intermediates 29-32 were prepared similarly to intermediate 28, by bromination of the appropriate intermediate with NBS; see Table 4 below.

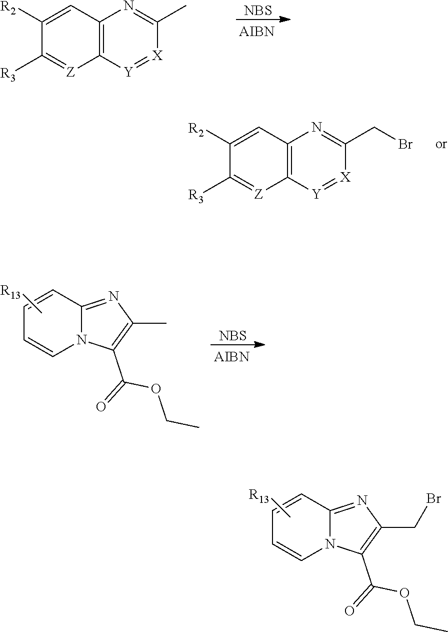

TABLE 4
Bromination of methyl heterocycles

| | | | Intermediate, Form, |
|---|---|---|---|
| Int | Structure | Name | Yield, LCMS, UPLC |
| 29 | 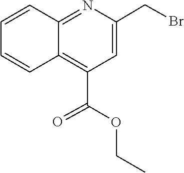 [SMILES: CCOC(=O)c1cc(CBr)nc2ccccc12] | Ethyl 2- (bromomethyl)quinoline- 4-carboxylate | From Ethyl 2- methylquinoline-4- carboxylate Red solid Yield 110 mg, 17.9% LCMS (ES+): 294.1 [MH]⁺. |
| 30 | 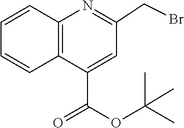 [SMILES: CC(C)(C)OC(=O)c1cc(CBr)nc2ccccc12] | tert-Butyl 2- (bromomethyl) quinoline-4-carboxylate | From Intermediate 18 Light-yellow oil Yield 328 mg, 37.5% LCMS (ES+): 322.0 [MH]⁺. UPLC (Method A) Rt 6.85 min, 99.1%. |
| 31 | 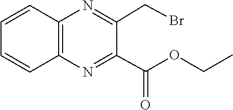 [SMILES: CCOC(=O)c1nc2ccccc2nc1CBr] | Ethyl 3-(bromomethyl) quinoxaline-2- carboxylate | From ethyl 3- methylquinoxaline-2- carboxylate Pink solid Yield 264 mg, 28.4% LCMS (ES+): 295.0 [MH]⁺. UPLC (Method B) Rt 2.75 min, 73.5%. |
| 32 | 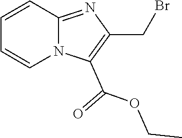 [SMILES: CCOC(=O)c1c(CBr)nc2ccccn12] | Ethyl 2- (bromomethyl)imidazo [1,2-a]pyridine-3- carboxylate | From ethyl 2- methylimidazo[1,2- a]pyridine-3-carboxylate Beige solid Yield 323 mg, 37.4% LCMS (ES+): 282.9 [MH]⁺. UPLC (Method B) Rt 2.26 min, 80.2%. |

### INTERMEDIATE 33

### Methyl 2-(bromomethyl)quinazoline-4-carboxylate

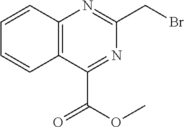 [SMILES: COC(=O)c1nc(CBr)nc2ccccc12]

Oxalyl chloride (0.19 mL, 2.23 mmol) was added to a solution of 2-methylquinazoline-4-carboxylic acid hydrochloride (250 mg, 1.11 mmol) in DCM (11 mL) and DMF (10 μL) at 0° C. The reaction was stirred for 30 min. MeCOH (1.0 mL) was added and the reaction was warmed to room temperature and stirred for 30 min. The mixture was concentrated in vacuo, diluted with EtOAc (30 mL), washed with sat aq NaHCO₃ (30 mL) dried (MgSO₄) and concentrated in vacuo. The residue was dissolved in CCl₄ (5.0 mL) and the mixture was sparged with N₂ for 5 min. Azobisisobutyronitrile (15.5 mg, 9.45 μmol) and NBS (210 mg, 1.18 mmol) were added and the reaction was heated under reflux for 20 h. The mixture filtered and concentrated in vacuo. The residue was purified by normal phase column chromatography to give the title compound (58.0 mg, 20.0%) as a white solid. UPLC (Method B) Rt 2.46 min, 91.5%. LCMS (ES+): 281.0 [MH]⁺.

### INTERMEDIATE 34

### Ethyl 2-(chloromethyl)-1,5-naphthyridine-3-carboxylate

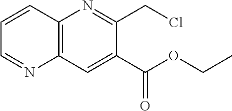 [SMILES: CCOC(=O)c1cc2ncccc2nc1CCl]

3-Aminopicolinaldehyde (500 mg, 4.09 mmol) and ethyl 4-chloro-3-oxobutanoate (0.66 mL, 4.91 mmol) were dissolved in EtOH (27 mL) and heated under reflux for 18 h. The mixture was concentrated in vacuo. The residue was separated between EtOAc (100 mL) and water (100 mL), the aq portion was extracted with EtOAc (100 mL) and the combined organics were dried (MgSO₄) and concentrated in vacuo. The residue purified by trituration in iso-hexane to give the title compound (724 mg, 69.5%) as a brown solid. UPLC (Method B) Rt 2.46 min, 98.5%. LCMS (ES+): 251.0 [MH]⁺.

### INTERMEDIATE 35

### Ethyl 2-(bromomethyl)-7-fluoro-quinoline-3-carboxylate

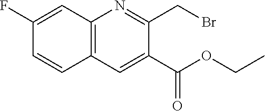 [SMILES: CCOC(=O)c1cc2ccc(F)cc2nc1CBr]

To a stirred mixture of intermediate 19 (757 mg, 3.25 mmol) and benzoyl peroxide (83.2 mg, 0.33 mmol) in CCl₄ (15 mL) were added NBS (520 mg, 2.92 mmol) and stirred at 80° C. for 2 days. The mixture was allowed to cool to room temperature, quenched with sat aq Na₂S₂O₃ and extracted with DCM (3×20 mL). The combined organic layers were washed with water (3×10 mL), dried (Na₂SO₄) and concentrated in vacuo. The residue was purified by reverse phase column chromatography to give the title compound (540 mg, 53.3%)* as a white solid. LCMS (ES+): 312.1 [MH]⁺.

### INTERMEDIATE 36 to 42

Intermediates 36-42 were prepared similarly to intermediate 35, by bromination of intermediates 20-24 and 26-27 with NBS; see Table 5 below.

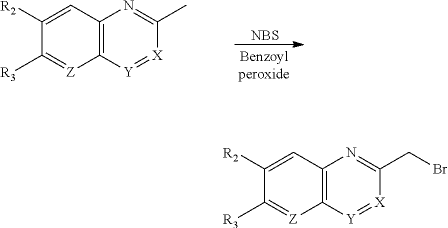

TABLE 5
Bromination of methyl heterocycles

| | | | Intermediate, Form, |
|---|---|---|---|
| Int | Structure | Name | Yield, LCMS |
| 36 | 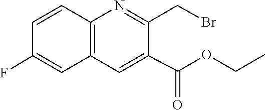 [SMILES: CCOC(=O)c1cc2cc(F)ccc2nc1CBr] | Ethyl 2- (bromomethyl)-6- fluoro-quinoline-3- carboxylate | From Intermediate 20 Yellow solid Yield 170 mg, 19.0%* LCMS (ES+): 312.0 [MH]⁺ |
| 37 | 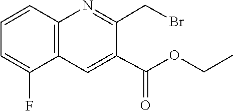 [SMILES: CCOC(=O)c1cc2c(F)cccc2nc1CBr] | Ethyl 2- (bromomethyl)-5- fluoro-quinoline-3- carboxylate | From Intermediate 21 Yellow solid Yield 1.1 g, 42.2%* LCMS (ES+): 312.2 [MH]⁺ |
| 38 | 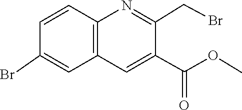 [SMILES: COC(=O)c1cc2cc(Br)ccc2nc1CBr] | Methyl 6-bromo-2- (bromomethyl) quinoline-3- carboxylate | From Intermediate 22 White solid Yield 1.4 g, 48.2%* LCMS (ES+): 360.2 [MH]⁺ |
| 39 | 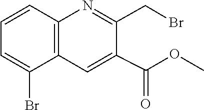 [SMILES: COC(=O)c1cc2c(Br)cccc2nc1CBr] | Methyl 5-bromo-2- (bromomethyl) quinoline-3- carboxylate | From Intermediate 23 Light yellow solid Yield 300 mg, 46.8%* LCMS (ES+): 360.2 [MH]⁺ |
| 40 | 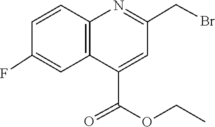 [SMILES: CCOC(=O)c1cc(CBr)nc2ccc(F)cc12] | Ethyl 2- (bromomethyl)-6- fluoro-quinoline-4- carboxylate | From Intermediate 24 Yellow solid Yield 115 mg, 28.6%* LCMS (ES+): 312.0 [MH]⁺. |
| 41 | 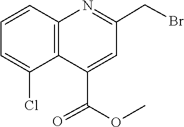 [SMILES: COC(=O)c1cc(CBr)nc2cccc(Cl)c12] | Methyl 2- (bromomethyl)-5- chloro-quinoline- 4-carboxylate | From Intermediate 26 Yellow solid Yield 80 mg, 35.3%* LCMS (ES+): 315.5 [MH]⁺. |
| 42 | 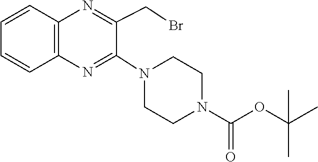 [SMILES: CC(C)(C)OC(=O)N1CCN(c2nc3ccccc3nc2CBr)CC1] | tert-Butyl 4-[3- (bromomethyl) quinoxalin-2- yl]piperazine-1- carboxylate | From Intermediate 27 Yellow solid Yield 30.0 mg, 17.3%* LCMS (ES+): 407.1 [MH]⁺. |

### 4-[2-(Chloromethyl)quinazolin-4-yl]-1,4-thiazinane 1,1-dioxide

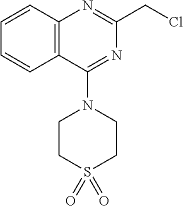 [SMILES: O=S1(=O)CCN(c2nc(CCl)nc3ccccc23)CC1]

2-(Chloromethyl)-3H-quinazolin-4-one (500 mg, 2.57 mmmol) was dissolved in POCl₃ (12 mL, 129 mmol). DIPEA (0.3 mL, 1.72 mmol) was added at 0° C. under N₂ and the reaction heated at 100° C. for 3 h. The mixture was allowed to cool then concentrated in vacuo. The residue was dissolved in dioxane (20 mL), thiomorpholine-1,1-dioxide (485 mg, 3.59 mmol) and DIPEA (619 mg, 4.79 mmol) were added at 0° C. under N₂ then heated at 60° C. for 2 days. The resulting mixture was extracted with EtOAc (3×20 mL), the combined organic layers were washed with brine (3×10 mL), dried (Na₂SO₄) then concentrated in vacuo. The residue was purified by silica gel column chromatography to give the title compound (145 mg, 19.4%)* as a dark red solid. LCMS (ES+): 312.1 [MH]⁺.

### INTERMEDIATES 44 and 45

Intermediates 44-45 were prepared similarly to intermediate 43, by chlorination followed by SNAr with the appropriate amine; see Table 6 below.

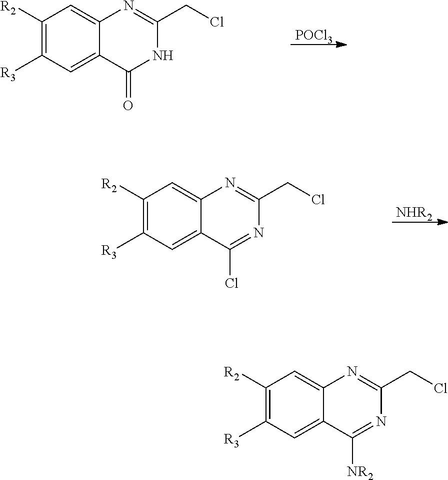

In which group “NR₂” may be

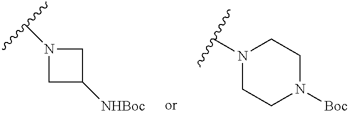 [SMILES: *N1CC(NC(=O)OC(C)(C)C)C1 ; *N1CCN(C(=O)OC(C)(C)C)CC1]

TABLE 6
Chlorination followed by SNAr

| | | | Amine, Form, |
|---|---|---|---|
| Int | Structure | Name | Yield, LCMS |
| 44 | 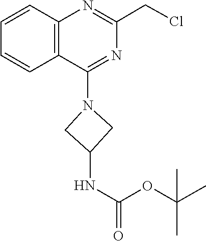 [SMILES: CC(C)(C)OC(=O)NC1CN(c2nc(CCl)nc3ccccc23)C1] | tert-Butyl N-[1-[2- (chloromethyl) quinazolin-4- ylazetidin-3- yl]carbamate | From tert-butyl N-(azetidin- 3-yl) carbamate Light yellow solid Yield 150 mg, 16.9%* LCMS 349.0 [MH]⁺ |
| 45 | 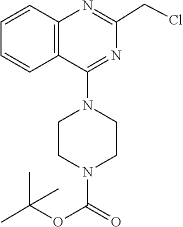 [SMILES: CC(C)(C)OC(=O)N1CCN(c2nc(CCl)nc3ccccc23)CC1] | tert-Butyl 4-[2- (chloromethyl) quinazolin-4- yl]piperazine-1- carboxylate | From tert-butyl piperazine-1- carboxylate Yellow solid Yield 510 mg, 39.4%* LCMS 363.2 [MH]⁺ |

### INTERMEDIATE 46

### tert-Butyl N-[1-(3-methylquinoxalin-2-yl)azetidin-3-yl]carbamate

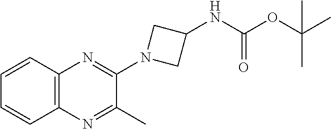 [SMILES: Cc1nc2ccccc2nc1N1CC(NC(=O)OC(C)(C)C)C1]

To a stirred solution of 2-chloro-3-methylquinoxaline (530 mg, 2.97 mmol) and tert-butyl N-(azetidin-3-yl)carbamate (511 mg, 2.97 mmol) in DMF was added DIPEA (959 mg, 7.42 mmol) dropwise and stirred for 3 h. The reaction was quenched with Water at room temperature, extracted with EtOAc (2×100 mL). The combined organic layers were concentrated in vacuo. The residue was purified by prep-TLC to give the title compound (500 mg, 53.6%)* as a yellow solid. LCMS (ES+): 315.2 [MH]⁺.

### INTERMEDIATE 47

### tert-Butyl N-[1-[3-(hydroxymethyl)quinoxalin-2-yl]azetidin-3-yl]carbamate

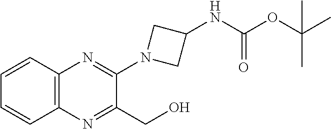 [SMILES: CC(C)(C)OC(=O)NC1CN(c2nc3ccccc3nc2CO)C1]

To a stirred solution of intermediate 46 (200 mg, 0.64 mmol) in 1,4-dioxane was added SeO₂ (141 mg, 1.27 mmol) and H₂O (115 mg, 6.36 mmol) dropwise then heated at 60° C. overnight. The reaction was quenched with water, was extracted with EtOAc (2×30 mL), the combined organic layers were concentrated in vacuo. The residue was dissolved in THF:MeOH (1:1, 3.0 mL) then NaBH(OAc)₃ (238 mg, 1.13 mmol) added and stirred for 20 min. The reaction was quenched with water, extracted with EtOAc (2×30 mL), the combined organic layers were concentrated in vacuo then purified by prep-TLC to give the title compound (110 mg, 59.1%)* as a yellow solid. LCMS (ES+): 331.2 [MH]⁺.

### INTERMEDIATE 48

### 2-[[4-[4-(4-Pyridyl)-1H-pyrazol-3-yl]phenoxy]methyl]quinoline

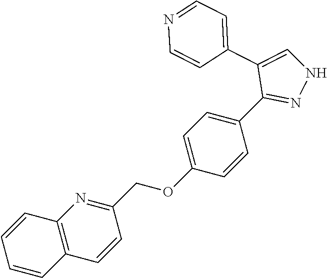 [SMILES: c1ccc2nc(COc3ccc(-c4n[nH]cc4-c4ccncc4)cc3)ccc2c1]

A mixture of intermediate 13 (100 mg, 96.2% pure, 285 μmol), 2-bromomethylquinoline (69.7 mg, 314 μmol) and Cs₂CO₃ (102 mg, 314 μmol) in DMF (3.0 mL) was stirred for 1 h. 1M aq HCl (3.0 mL) was added and the reaction was stirred for 3 days. The reaction mixture was partitioned between DCM (50 mL) and sat aq NaHCO₃ (50 mL). The aq layer was extracted with DCM (50 mL) and the organic layers combined, dried (MgSO₄) and concentrated in vacuo. The residue was purified by reverse phase HPLC to give the title compound (37.4 mg, 34.5%) as a white solid. UPLC (Method A) Rt 3.37 min, 99.6%. LCMS (ES+): 379.1 [MH]⁺.

### INTERMEDIATE 49

### Ethyl 2-[4-(4-pyridyl)-3-[4-(2-quinolylmethoxy)phenyl]pyrazol-1-yl]acetate

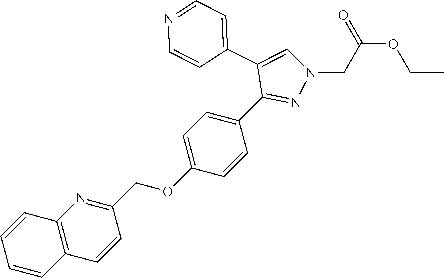 [SMILES: CCOC(=O)Cn1cc(-c2ccncc2)c(-c2ccc(OCc3ccc4ccccc4n3)cc2)n1]

Ethyl bromoacetate (47.7 μL, 431 μmol) was added to a suspension of intermediate 48 (150 mg, 98.9% pure, 392 μmol), K₂CO₃ (65.0 mg, 470 μmol) and tetrabutylammonium iodide (14.5 mg, 39.2 μmol) in DMF (4.0 mL), and then heated at 80° C. for 1 h. The reaction was diluted with water (50 mL) and extracted with EtOAc (2×50 mL). The combined organics were washed with water (2×50 mL), brine (50 mL), dried (MgSO₄) and concentrated in vacuo. The residue was purified by normal phase column chromatography to give the title compound (134 mg, 35.8%) as an orange solid. UPLC (Method B) Rt 2.16 min, 48.6%. LCMS (ES+): 465.2 [MH]⁺.

### INTERMEDIATE 50

### Methyl 2-[[4-[1-methyl-4-(4-pyridyl)pyrazol-3-yl]phenoxy]methyl]quinoline-3-carboxylate

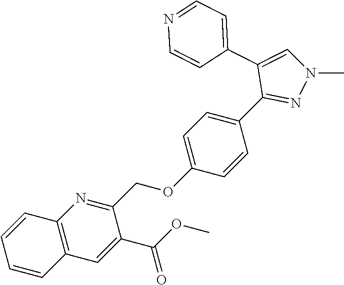 [SMILES: COC(=O)c1cc2ccccc2nc1COc1ccc(-c2nn(C)cc2-c2ccncc2)cc1]

Under N₂ a solution of intermediate 11 (300 mg, 96.1% pure, 1.15 mmol) in DMF (4.0 mL) was added dropwise to a suspension of NaH (60% in mineral oil, 50.5 mg, 1.26 mmol) in DMF (8.0 mL) at 0° C. and stirred for 30 min. Intermediate 28 (345 mg, 93.2% pure, 1.15 mmol) was added and the mixture was allowed to warm to room temperature over 16 h. The reaction mixture was partitioned between DCM (100 mL), H₂O (100 mL) and brine (50 mL), the aq layer extracted with DCM (100 mL) and the organic layers combined, washed with brine (100 mL), dried (MgSO₄) and concentrated in vacuo. The residue was purified by normal phase column chromatography to give the title compound (439 mg, 82.5%) as a yellow solid. UPLC (Method A), Rt 4.52 min, 97.1%. LCMS (ES+): 451.2 [MH]⁺.

### INTERMEDIATES 51 to 64

Intermediates 51-64 were prepared similarly to intermediate 50, by alkylation of the appropriate phenol intermediates with the appropriate bromide intermediates using NaH; see Table 7 below.

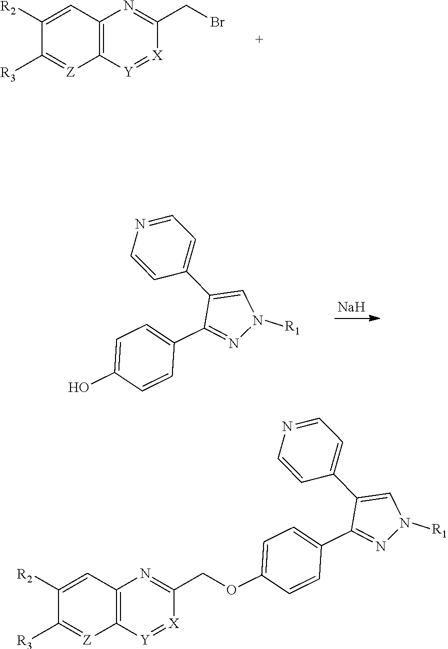

TABLE 7
Alkylation reactions using NaH as a base

| | | | Intermediate(s), |
|---|---|---|---|
| | | | Form, |
| | | | Yield, LCMS, |
| Int | Structure | Name | UPLC |
| 51 | 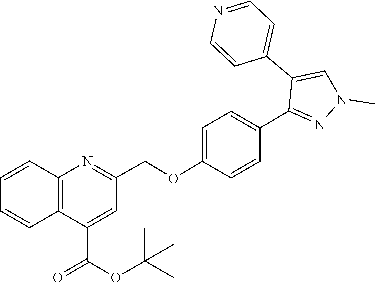 [SMILES: Cn1cc(-c2ccncc2)c(-c2ccc(OCc3cc(C(=O)OC(C)(C)C)c4ccccc4n3)cc2)n1] | tert-Butyl 2-[[4-[1- methyl-4-(4- pyridyl)pyrazol-3- yl]phenoxy]methyl] quinoline-4- carboxylate | From Intermediates 11 and 30 Yellow gum Yield 500 mg, 90.1% LCMS (ES+): 493.3 [MH]⁺. UPLC (Method A), Rt 5.23 min, 89.5%. |
| 52 | 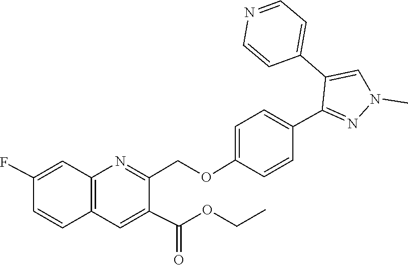 [SMILES: CCOC(=O)c1cc2ccc(F)cc2nc1COc1ccc(-c2nn(C)cc2-c2ccncc2)cc1] | Ethyl 7-fluoro-2-[[4- [1-methyl-4-(4- pyridyl)pyrazol-3- yl]phenoxy]methyl] quinoline-3- carboxylate | From Intermediates 11 and 35 Yellow solid Yield 110 mg, 57.3%* LCMS (ES+): 483.1 [MH]⁺ |
| 53 | 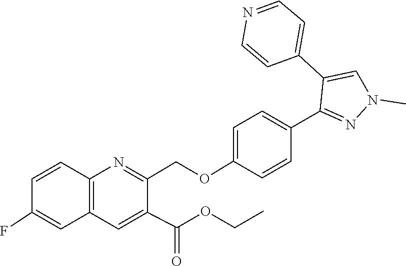 [SMILES: CCOC(=O)c1cc2cc(F)ccc2nc1COc1ccc(-c2nn(C)cc2-c2ccncc2)cc1] | Ethyl 6-fluoro-2-[[4- [1-methyl-4-(4- pyridyl)pyrazol-3- yl]phenoxy]methyl] quinoline-3- carboxylate | From Intermediates 11 and 36 Yellow solid Yield 200 mg, 76.1%* LCMS (ES+): 483.2 [MH]⁺ |
| 54 | 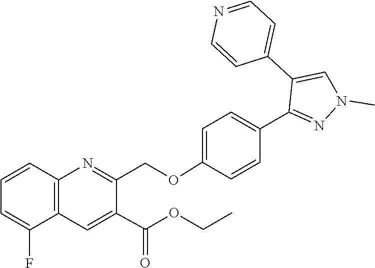 [SMILES: CCOC(=O)c1cc2c(F)cccc2nc1COc1ccc(-c2nn(C)cc2-c2ccncc2)cc1] | Ethyl 5-fluoro-2-[[4- [1-methyl-4-(4- pyridyl)pyrazol-3- yl]phenoxy]methyl] quinoline-3- carboxylate | From Intermediates 11 and 37 White solid Yield 116 mg, 60.4%* LCMS (ES+): 482.9 [MH]⁺ |
| 55 | 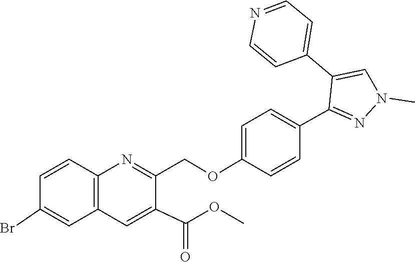 [SMILES: COC(=O)c1cc2cc(Br)ccc2nc1COc1ccc(-c2nn(C)cc2-c2ccncc2)cc1] | Methyl 6-bromo-2-[[4- [1-methyl-4-(4- pyridyl)pyrazol-3- yl]phenoxy]methyl] quinoline-3- carboxylate | From Intermediates 11 and 38 Yellow solid Yield 107 mg, 24.2%* LCMS (ES+): 529.1 [MH]⁺ |
| 56 | 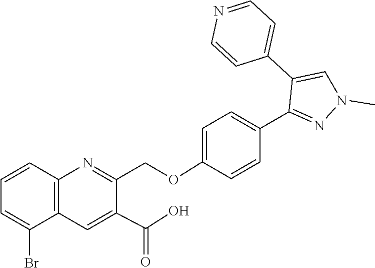 [SMILES: Cn1cc(-c2ccncc2)c(-c2ccc(OCc3nc4cccc(Br)c4cc3C(=O)O)cc2)n1] | 5-Bromo-2-[[4-[1- methyl-4-(4- pyridyl)pyrazol-3- yl]phenoxy]methyl] quinoline-3- carboxylic acid | From Intermediates 11 and 39 Light yellow solid Yield 150 mg, 52.3%* LCMS (ES+): 515.3 [MH]⁺ |
| 57 | 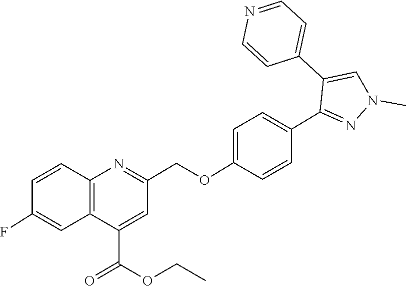 [SMILES: CCOC(=O)c1cc(COc2ccc(-c3nn(C)cc3-c3ccncc3)cc2)nc2ccc(F)cc12] | Ethyl 6-fluoro-2-[[4- [1-methyl-4-(4- pyridyl)pyrazol-3- yl]phenoxy]methyl] quinoline-4- carboxylate | From Intermediates 11 and 40 Off-white solid Yield 98.0 mg, 72.9%* LCMS (ES+): 483.2 [MH]⁺ |
| 58 | 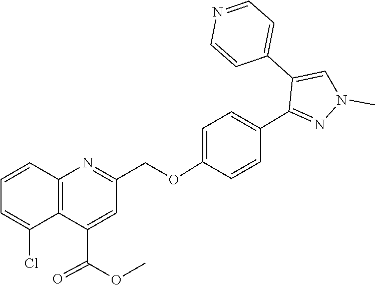 [SMILES: COC(=O)c1cc(COc2ccc(-c3nn(C)cc3-c3ccncc3)cc2)nc2cccc(Cl)c12] | Methyl 5-chloro-2-[[4- [1-methyl-4-(4- pyridyl)pyrazol-3- yl]phenoxy]methyl] quinoline-4- carboxylate | From Intermediates 11 and 41 Yellow solid Yield 80.0 mg, 92.1%* LCMS (ES+): 485.1 [MH]⁺. |
| 59 | 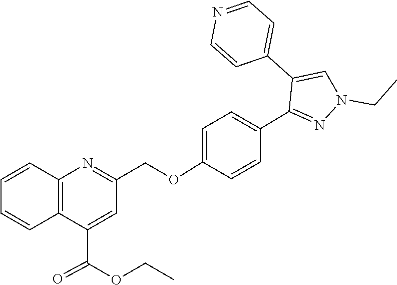 [SMILES: CCOC(=O)c1cc(COc2ccc(-c3nn(CC)cc3-c3ccncc3)cc2)nc2ccccc12] | Ethyl 2-[[4-[1-ethyl-4- (4-pyridyl)pyrazol-3- yl]phenoxy]methyl] quinoline-4- carboxylate | From Intermediates 15 and 29 Off-white solid Yield 35.0 mg, 38.8%* LCMS (ES+): 479.2 [MH]⁺ |
| 60 | 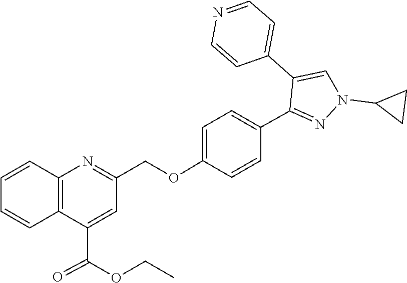 [SMILES: CCOC(=O)c1cc(COc2ccc(-c3nn(C4CC4)cc3-c3ccncc3)cc2)nc2ccccc12] | Ethyl 2-[[4-[1- cyclopropyl-4-(4- pyridyl)pyrazol-3- yl]phenoxy]methyl] quinoline-4- carboxylate | From Intermediates 16 and 29 Orange solid Yield 43.0 mg, Crude LCMS (ES+): 491.3 [MH]⁺ |
| 61 | 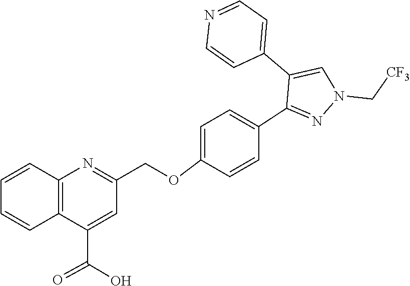 [SMILES: O=C(O)c1cc(COc2ccc(-c3nn(CC(F)(F)F)cc3-c3ccncc3)cc2)nc2ccccc12] | 2-[[4-[4-(4-Pyridyl)-1- (2,2,2- trifluoroethyl)pyrazol- 3-yl]phenoxy]methyl] quinoline-4- carboxylic acid | From Intermediates 17 and 29 Yellow solid Yield 45.0 mg, 45.2%* LCMS (ES+): 505.1 [MH]⁺ |
| 62 | 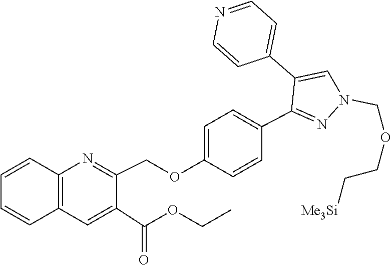 [SMILES: CCOC(=O)c1cc2ccccc2nc1COc1ccc(-c2nn(COCC[Si](C)(C)C)cc2-c2ccncc2)cc1] | Ethyl 2-[[4-[4-(4- pyridyl)-1-(2- trimethylsilylethoxy methyl)pyrazol-3- yl]phenoxy]methyl] quinoline-3- carboxylate | From Intermediates 14 and the ethyl ester equivalent of intermediate 28 Yellow solid Yield 31.0 mg, 98.1%* LCMS (ES+): 581.2 [MH]⁺. |
| 63 | 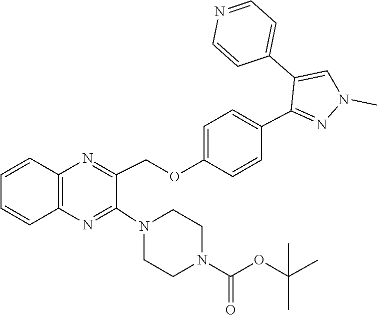 [SMILES: Cn1cc(-c2ccncc2)c(-c2ccc(OCc3nc4ccccc4nc3N3CCN(C(=O)OC(C)(C)C)CC3)cc2)n1] | tert-Butyl 4-[3-[[4-[1- methyl-4-(4- pyridyl)pyrazol-3- yl]phenoxy]methyl] quinoxalin-2- yl]piperazine-1- carboxylate | From Intermediates 11 and 42 Material used crude in the next step LCMS (ES+): 578.3 [MH]⁺. |
| 64 | 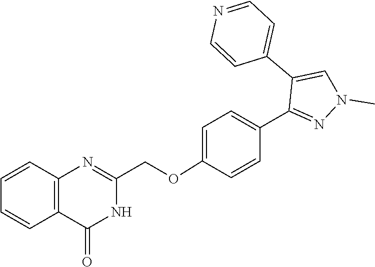 [SMILES: Cn1cc(-c2ccncc2)c(-c2ccc(OCc3nc4ccccc4c(=O)[nH]3)cc2)n1] | 2-[[4-[1-Methyl-4-(4- pyridyl)pyrazol-3- yl]phenoxy]methyl]- 3H-quinazolin-4-one | From Intermediate 11 Yellow solid Yield 30.0 mg, 9.21%* LCMS (ES+): 410.1 [MH]⁺. |

### INTERMEDIATE 65

### Methyl 2-[[4-[1-methyl-4-(4-pyridyl)pyrazol-3-yl]phenoxy]methyl]quinazoline-4-carboxylate

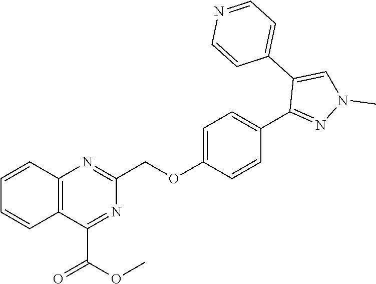 [SMILES: COC(=O)c1nc(COc2ccc(-c3nn(C)cc3-c3ccncc3)cc2)nc2ccccc12]

A mixture of intermediate 11 (56.1 mg, 99.2% pure, 0.22 mmol), intermediate 33 (68.0 mg, 91.5% pure, 0.22 mmol) and Cs₂CO₃ (79.3 mg, 0.24 mmol) in DMF (3.0 mL) was stirred for 16 h. The mixture was diluted with DCM (20 mL), washed with sat aq NaHCO₃ (20 mL), dried (MgSO₄) and concentrated in vacuo. The residue was purified by normal phase column chromatography to give the title compound (35.0 mg, 34.6%) as a yellow solid. UPLC (Method B) Rt 2.24 min, 98.7%. LCMS (ES+): 452.1 [MH]⁺.

### INTERMEDIATE 66 to 72

Intermediates 66-72 were prepared similarly to intermediate 65, by alkylation of the appropriate phenol intermediates with the appropriate bromide/chloride intermediates using Cs₂CO₃, see Table 8 below.

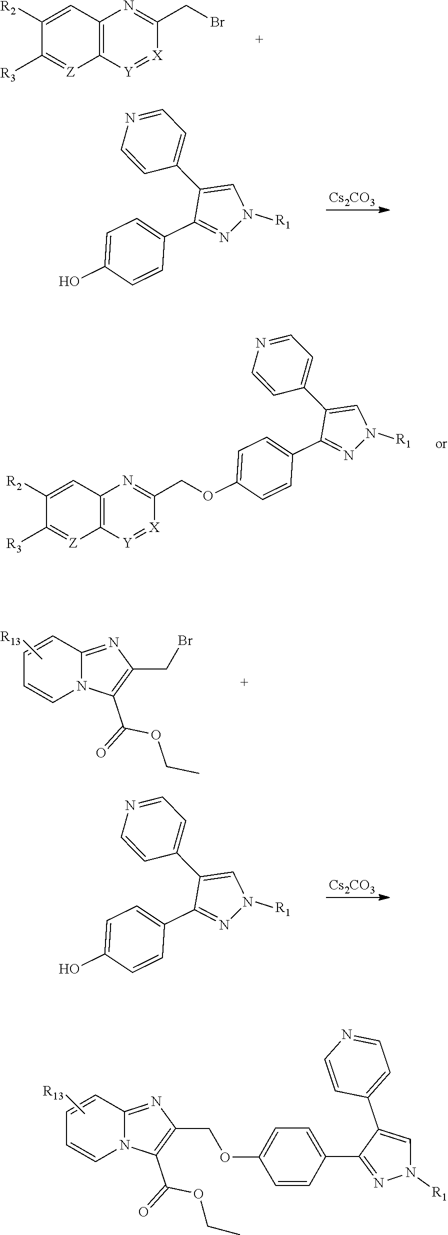

TABLE 8
Alkylation reactions using Cs₂CO₃ as a base

| | | | Intermediate(s), |
|---|---|---|---|
| | | | Form, |
| Int | Structure | Name | Yield, LCMS, UPLC |
| 66 | 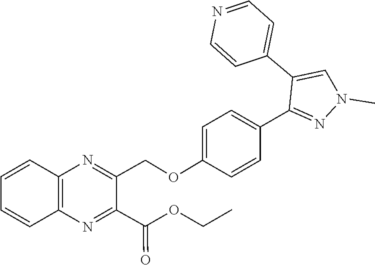 [SMILES: CCOC(=O)c1nc2ccccc2nc1COc1ccc(-c2nn(C)cc2-c2ccncc2)cc1] | Ethyl 3-[[4-[1- methyl-4-(4- pyridyl)pyrazol-3- yl]phenoxy] methyl] quinoxaline-2- carboxylate | From Intermediates 11 and 31 White solid Yield 47.0 mg, 28.2% LCMS (ES+): 466.2 [MH]⁺ UPLC (Method B): Rt 2.37 min, 93.8% |
| 67 | 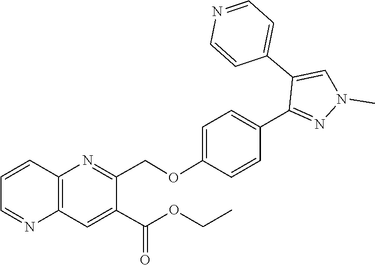 [SMILES: CCOC(=O)c1cc2ncccc2nc1COc1ccc(-c2nn(C)cc2-c2ccncc2)cc1] | Ethyl 2-[[4-[1- methyl-4-(4- pyridyl)pyrazol-3- yl]phenoxy] methyl]-1,5- naphthyridine-3- carboxylate | From Intermediates 11 and 34 Brown solid Yield 87.0 mg, 76.4% LCMS (ES+): 466.2 [MH]⁺ UPLC (Method B): Rt 2.24 min, 96.8% |
| 68 | 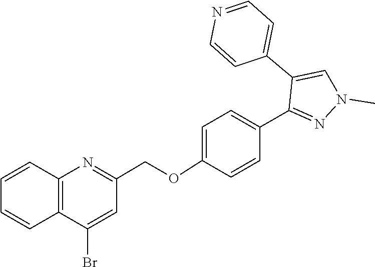 [SMILES: Cn1cc(-c2ccncc2)c(-c2ccc(OCc3cc(Br)c4ccccc4n3)cc2)n1] | 4-Bromo-2-[[4-[1- methyl-4-(4- pyridyl)pyrazol-3- yl]phenoxy] methyl]quinoline | From Intermediate 11 and bromo-2- (bromomethyl) quinoline Yellow gum Yield 431mg, 76.5% LCMS (ES+): 471.2 [MH]⁺. UPLC (Method A) Rt 4.94 min, 96.0% |
| 69 | 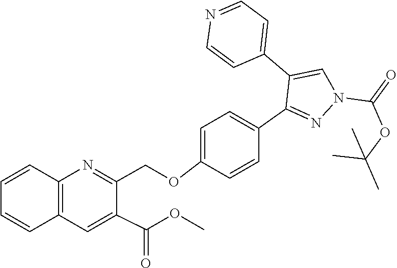 [SMILES: COC(=O)c1cc2ccccc2nc1COc1ccc(-c2nn(C(=O)OC(C)(C)C)cc2-c2ccncc2)cc1] | Methyl 2-[[4-[1- tert- butoxycarbonyl- 4-(4- pyridyl)pyrazol-3- yl]phenoxy] methyl]quinoline-3- carboxylate | From Intermediate 13 and 28 Yellow solid Yield 62.0 mg, 59.8% LCMS (ES+): 537.3 [MH]⁺ UPLC (Method A): Rt 5.41 min, 88.6% |
| 70 | 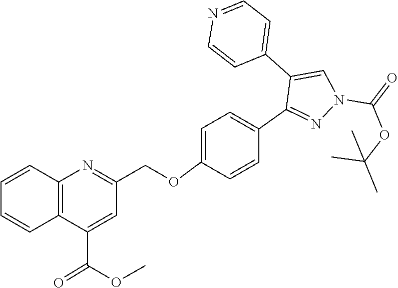 [SMILES: COC(=O)c1cc(COc2ccc(-c3nn(C(=O)OC(C)(C)C)cc3-c3ccncc3)cc2)nc2ccccc12] | Methyl 2-[[4-[1- tert- butoxycarbonyl- 4-(4- pyridyl)pyrazol-3- yl]phenoxy] methyl]quinoline-4- carboxylate | From Intermediates 13 and the methyl ester analogue of intermediate 29 Yellow liquid Yield 153 mg, 87.5% LCMS (ES+): 537.3 [MH]⁺ UPLC (Method A): Rt 5.57 min, 87.5% |
| 71 | 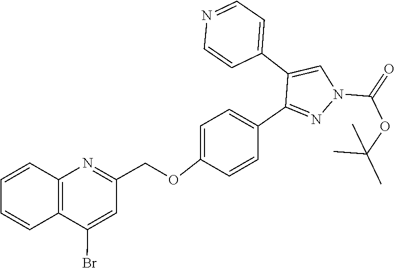 [SMILES: CC(C)(C)OC(=O)n1cc(-c2ccncc2)c(-c2ccc(OCc3cc(Br)c4ccccc4n3)cc2)n1] | tert-Butyl 3-[4-[(4- bromo-2- quinolyl)methoxy] phenyl]-4-(4- pyridyl)pyrazole- 1-carboxylate | From Intermediate 13 and bromo-2- (bromomethyl) quinoline Yellow solid Yield 153 mg, 87.5% LCMS (ES+): 557.1 [MH]⁺. UPLC (Method A) Rt 5.91 min, 93.1% |
| 72 | 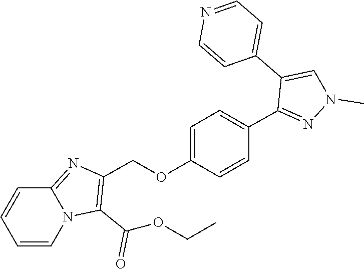 [SMILES: CCOC(=O)c1c(COc2ccc(-c3nn(C)cc3-c3ccncc3)cc2)nc2ccccn12] | Ethyl 2-[[4-[1- methyl-4-(4- pyridyl)pyrazol-3- yl]phenoxy] methyl]imidazo [1,2-a]pyridine-3- carboxylate | From Intermediates 11 and 32 Yellow solid Yield 86 mg, 43.0% LCMS (ES+): 454.1 [MH]⁺. UPLC (Method B) Rt 2.10 min, 89.6% |

### INTERMEDIATE 73

### tert-Butyl N-[1-[2-[[4-[1-methyl-4-(4-pyridyl)pyrazol-3-yl]phenoxy]methyl]quinazolin-4-yl]azetidin-3-yl]carbamate

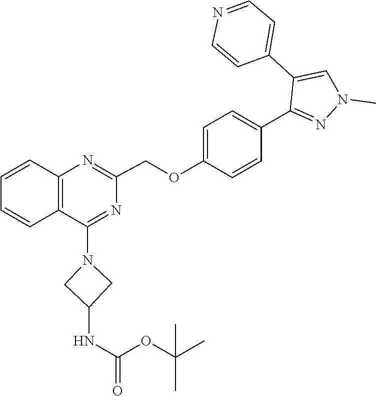 [SMILES: Cn1cc(-c2ccncc2)c(-c2ccc(OCc3nc(N4CC(NC(=O)OC(C)(C)C)C4)c4ccccc4n3)cc2)n1]

A mixture of intermediate 44 (50.0 mg, 0.14 mmol) and intermediate 11 (36.0 mg, 0.14 mmol) and K₂CO₃ (39.6 mg, 0.29 mmol) in MeCN (2.0 mL) was heated at 80° C. for 2 h. The reaction was concentrated in vacuo then purified by prep-TLC to Give the title compound (59.1 mg, 73.2%)* as a light yellow solid. LCMS (ES+): 564.3 [MH]⁺.

### INTERMEDIATE 74

### tert-Butyl 4-[2-[[4-[1-methyl-4-(4-pyridyl)pyrazol-3-yl]phenoxy]methyl]quinazolin-4-yl]piperazine-1-carboxylate

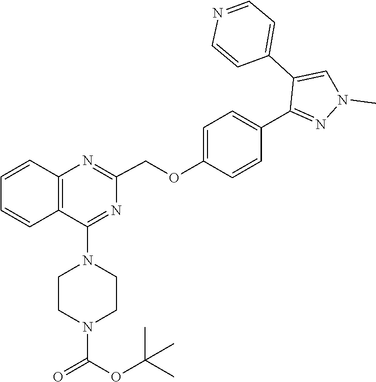 [SMILES: Cn1cc(-c2ccncc2)c(-c2ccc(OCc3nc(N4CCN(C(=O)OC(C)(C)C)CC4)c4ccccc4n3)cc2)n1]

Intermediate 74 was prepared similarly to intermediate 73, by alkylation of intermediate 11 with intermediate 45 to give the title compound (28.0 mg, 40.6%)* as a brown oil. LCMS (ES+): 578.3 [MH]⁺

### INTERMEDIATE 75

### Methyl 6-methyl-2-[[4-[1-methyl-4-(4-pyridyl)pyrazol-3-yl]phenoxy]methyl]quinoline-3-carboxylate

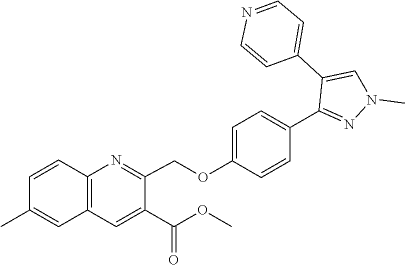

To a solution of intermediate 55 (100 mg, 0.19 mmol) and potassium difluoro(methyl)borane fluoride (23.0 mg, 0.19 mmol) in anhydrous dioxane (3.0 mL) was added Cs₂CO₃ (123 mg, 0.38 mmol) and Pd(dppf)Cl₂·DCM (15.0 mg, 0.02 mmol) the reaction heated at 80° C. for 16 h under N₂. The reaction was concentrated in vacuo then purified by silica gel column chromatography to give the title compound (120 mg) as a yellow solid. The material was taken into the next reaction without any further purification. LCMS (ES+): 465.15 [MH]⁺

### INTERMEDIATE 76

### 5-Methyl-2-[[4-[1-methyl-4-(4-pyridyl)pyrazol-3-yl]phenoxy]methyl]quinoline-3-carboxylic acid

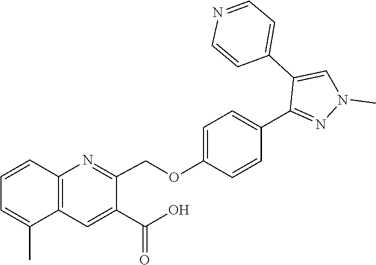 [SMILES: Cc1cccc2nc(COc3ccc(-c4nn(C)cc4-c4ccncc4)cc3)c(C(=O)O)cc12]

A mixture of intermediate 56 (100 mg, 0.19 mmol) and potassium trifluoro (methyl)boranuide (35.0 mg, 0.29 mmol) and Pd(PPh₃)₂Cl₂ (27.0 mg, 0.04 mmol) and Cs₂CO₃ (126 mg, 0.39 mmol) in anhydrous 1,4-dioxane was stirred at 100° C. under N₂ overnight. The resulting mixture was filtered, the filter cake washed with 1,4-dioxane (2×5 mL) and concentrated in vacuo. The residue was purified by reverse phase column chromatography to give the title compound (20.0 mg, 22.9%) as a yellow oil. LCMS (ES+): 451.1 [MH]⁺.

### INTERMEDIATE 77

### tert-Butyl N-[1-[3-[[4-[1-methyl-4-(4-pyridyl)pyrazol-3-yl]phenoxy]methyl]quinoxalin-2-yl]azetidin-3-yl]carbamate

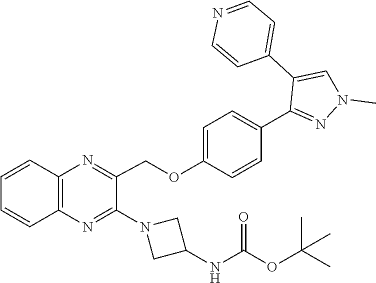 [SMILES: Cn1cc(-c2ccncc2)c(-c2ccc(OCc3nc4ccccc4nc3N3CC(NC(=O)OC(C)(C)C)C3)cc2)n1]

To a stirred solution of intermediate 47 (45.0 mg, 0.14 mmol), intermediate 11 (51.3 mg, 0.20 mmol) and PPh₃ (53.6 mg, 0.20 mmol) in anhydrous THF was added diisopropyl azodicarboxylate (41.3 mg, 0.20 mmol) dropwise at 0° C. under N₂ and stirred overnight. The reaction was quenched with water, extracted with EtOAc (2×30 mL), the organic layers were concentrated in vacuo then purified by prep-TLC to give the title compound (40.0 mg, 52.1%)* as a yellow solid. LCMS (ES+): 564.3 [MH]⁺.

### INTERMEDIATE 78

### 4-Methylsulfanyl-2-[[4-[4-(4-pyridyl)-1H-pyrazol-3-yl]phenoxy]methyl]quinoline

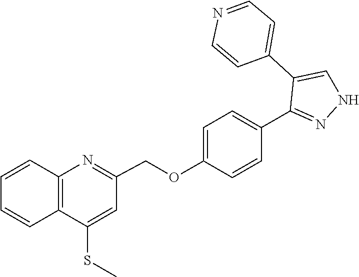 [SMILES: CSc1cc(COc2ccc(-c3n[nH]cc3-c3ccncc3)cc2)nc2ccccc12]

A solution of intermediate 71 (154 mg, 93.1% pure, 257 μmol) in DMF (2.6 mL) was sparged with N₂ for 5 min. Sodium thiomethoxide (39.7 μmg, 566 μmol) was added and the reaction was stirred at 100° C. for 19 h. The reaction mixture was partitioned between EtOAc (20 mL) and H₂O (20 mL). The aq layer was extracted with EtOAc (20 mL), the organic layers combined, dried (MgSO₄) and concentrated in vacuo to give the title compound (109 mg, 80.6%) as a yellow solid. UPLC (Method A) Rt 3.52 min, 80.7%. LCMS (ES+): 425.1 [MH]⁺.

### INTERMEDIATE 79

### 4-Methylsulfinyl-2-[[4-[4-(4-pyridyl)-1H-pyrazol-3-yl]phenoxy]methyl]quinoline

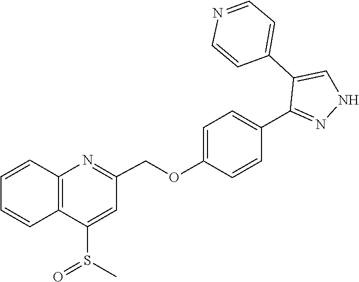 [SMILES: CS(=O)c1cc(COc2ccc(-c3n[nH]cc3-c3ccncc3)cc2)nc2ccccc12]

3-Chloroperbenzoic acid (61.3 mg, 70-75% pure, 249 μmol) was added portion wise to a solution of intermediate 78 (109 mg, 80.7% pure, 207 μmol) in DCM (4.1 mL) at 0° C. and the reaction was stirred for 10 min. The reaction was warmed to room temperature and stirred for 1 h. The reaction mixture was partitioned between DCM (20 mL) and sat aq NaHCO₃ (20 mL). The aq layer was extracted with DCM (20 mL) and the organic layers combined, dried (MgSO₄) and concentrated in vacuo to give the title compound (91.0 mg, 61.1%) as a yellow solid. UPLC (Method A) Rt 3.62 min, 61.3%. LCMS (ES+): 441.0 [MH]⁺.

### INTERMEDIATE 80

### N-[2-[[4-[1-Methyl-4-(4-pyridyl)pyrazol-3-yl]phenoxy]methyl]-4-quinolyl]-1,1-diphenyl-methanimine

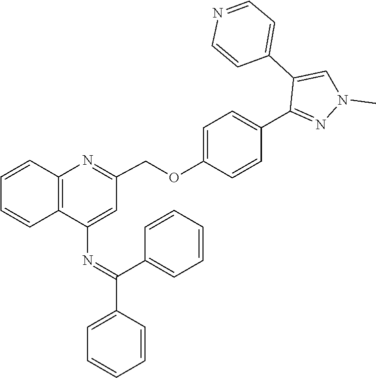 [SMILES: Cn1cc(-c2ccncc2)c(-c2ccc(OCc3cc(N=C(c4ccccc4)c4ccccc4)c4ccccc4n3)cc2)n1]

To a stirred solution of diphenylmethanimine (61.5 mg, 0.34 mmol), intermediate 68 (80.0 mg, 0.17 mmol), K₂CO₃ (46.9 mg, 0.34 mmol) in dioxane (3.0 mL) was added Pd₂(dba)₃·CHCl₃ (17.6 mg, 0.02 mmol) and XPhos (16.2 mg, 0.03 mmol) in portions under N₂ then heated at 100° C. overnight. The reaction was concentrated in vacuo then purified by prep-TLC to give the title compound (70.0 mg, 72.1%)* as a yellow solid. LCMS (ES+): 572.4 [MH]⁺.

### INTERMEDIATE 81

### N-[Dimethyl(oxo)-λ\hat{ }{6}-sulfanylidene]-2-[[4-[4-(4-pyridyl)-1-(2-trimethylsilylethoxymethyl)pyrazol-3-yl]phenoxy]methyl]quinoline-3-carboxamide

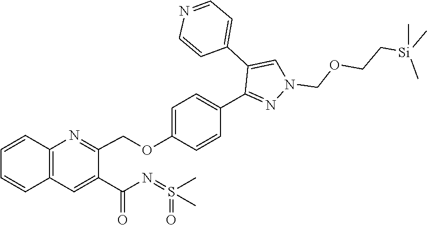 [SMILES: C[Si](C)(C)CCOCn1cc(-c2ccncc2)c(-c2ccc(OCc3nc4ccccc4cc3C(=O)N=S(C)(C)=O)cc2)n1]

Intermediate 62 (32.0 mg, 0.06 mmol) was dissolved in THF:H₂O (1:1), LiOH (4.00 mg, 0.17 mmol) was added and was stirred for 3 h. The mixture was acidified to pH 5 with aq HCl, extracted with EtOAc (3×5 mL), the combined organic layers were washed with brine (3×5 mL), dried (Na₂SO₄) and concentrated in vacuo. The residue was dissolved in DCM (5.0 mL), S,S-dimethyl sulfoximine (8.40 mg, 0.09 mmol), DMAP (6.60 mg, 0.05 mmol), 2-chloro-1-methylpyridin-1-ium iodide (23.1 mg, 0.09 mmol) and DIPEA (17.5 mg, 0.14 mmol) were added and the reaction stirred for 2 h. The reaction was quenched with water, extracted with DCM (3×5 mL), the combined organic layers were washed with brine (3×5 mL), dried (Na₂SO₄) and concentrated in vacuo. The residue was purified by Prep-TLC to give the title compound (15.0 mg, 52.8%)* as a yellow solid. LCMS (ES+): 628.3 [MH]⁺.

### Example 1

### 2-[[4-[2-Methyl-4-(4-pyridyl)pyrazol-3-yl]phenoxy]methyl]quinoline-4-carboxylic acid

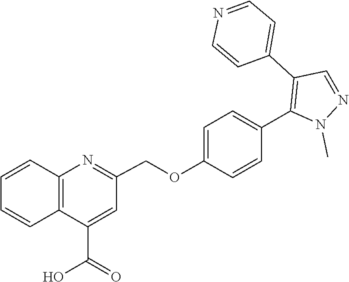 [SMILES: Cn1ncc(-c2ccncc2)c1-c1ccc(OCc2cc(C(=O)O)c3ccccc3n2)cc1]

A mixture of intermediate 12 (150 mg, 0.60 mmol), methyl 2-(chloromethyl)quinoline-4-carboxylate (159 mg, 97.1% pure, 0.66 mmol), and Cs₂CO₃ (391 mg, 1.19 mmol) in DMF (3.0 mL) was stirred at 40° C. for 3 days. The reaction was diluted with DCM (20 mL), washed with water (2×10 mL) and concentrated in vacuo. The residue suspended in THF (5.0 mL), aq NaOH (2.00 mL, 1.0M, 2.00 mmol) was added and stirred at 30° C. overnight. The reaction was concentrated in vacuo and purified by reverse phase HPLC (formic acid buffered) then isolated using SPE on a Biotage SCX-II column to give the title compound (65.0 mg, 24.7%) as a pale-yellow solid. UPLC (Method A) Rt 3.92 min, 98.9%. HRMS (ES+/QToF) m/z: [M+H]⁺ Calcd for C₂₆H₂₁N₄O₃ 437.1614; Found 437.1608.

### Example 2

### 2-[[4-[2-Methyl-4-(4-pyridyl)pyrazol-3-yl]phenoxy]methyl]quinoline-3-carboxylic acid

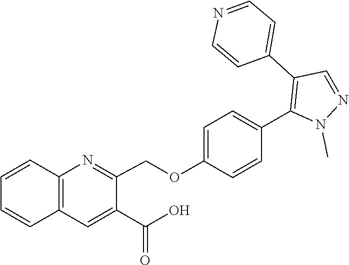 [SMILES: Cn1ncc(-c2ccncc2)c1-c1ccc(OCc2nc3ccccc3cc2C(=O)O)cc1]

Example 2 was prepared similarly to example 1, from intermediates 12 and 28 to give the title compound (35.5 mg, 13.4%) as a pale-yellow solid. LCMS (Method A) Rt 3.93 min, 98.6%. HRMS (ES+/QToF) m/z: [M+H]⁺ Calcd for C₂₆H₂₁N₄O₃ 437.1614; Found 437.1605.

### Example 3

### 2-[[4-[1-Methyl-4-(4-pyridyl)pyrazol-3-yl]phenoxy]methyl]quinoline-3-carboxylic acid

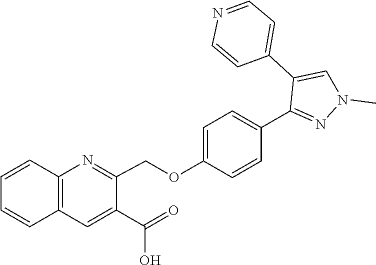 [SMILES: Cn1cc(-c2ccncc2)c(-c2ccc(OCc3nc4ccccc4cc3C(=O)O)cc2)n1]

LiOH·H₂O (119 mg, 2.84 mmol) was added to a solution of intermediate 50 (439 mg, 97.1% pure, 946 μmol) in THF (5.0 mL) and water (5.0 mL) and stirred for 2 h. The volatiles were removed in vacuo. To the remaining aq portion was added 1M aq HCl (2.84 mL). The resulting solid was collected by filtration and washed with water (2×5 mL) to give 2-[[4-[1-methyl-4-(4-pyridyl)pyrazol-3-yl]phenoxy]methyl]quinoline-3-carboxylic acid (370 mg, 87.8%) as a white solid. UPLC (Method A) Rt 3.78 min, 98.0%. HRMS (ES+/QToF) m/z: [M+H]⁺ Calcd for C₂₆H₂₁N₄O₃ 437.1614; Found 437.1615.

### Example 4

### Ammonium 2-[[4-[1-methyl-4-(4-pyridyl)pyrazol-3-yl]phenoxy]methyl]quinoline-4-carboxylate

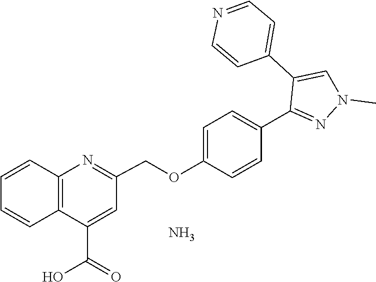

1,4-Dioxane (5.0 mL) and hydrochloric acid (4M in 1,4-dioxane, 5.0 mL, 20 mmol) was added to intermediate 51 (500 mg, 909 μmol) in water (5.0 mL) and the reaction heated at 60° C. for 3 h. The reaction mixture was concentrated in vacuo to give 2-[[4-[1-methyl-4-(4-pyridyl)pyrazol-3-yl]phenoxy]methyl]quinoline-4-carboxylic acid dihydrochloride (510 mg, 99.8%) as a brown solid. 50 mg was dissolved in THF (2.0 mL) and water (2.0 mL) and neutralised to pH 7 then purified by reverse phase HPLC (ammonia buffered) to give the title compound (14.8 mg, 3.58%) as a white solid. UPLC (Method A), Rt 3.77 min, 99.6%. HRMS (ES+/QToF) m/z: [M+H]⁺ Calcd for C₂₆H₂₁N₄O₃ 437.1614; Found 437.1611.

### Example 5

### 2-[4-(4-Pyridyl)-3-[4-(2-quinolylmethoxy)phenyl]pyrazol-1-yl]acetic acid

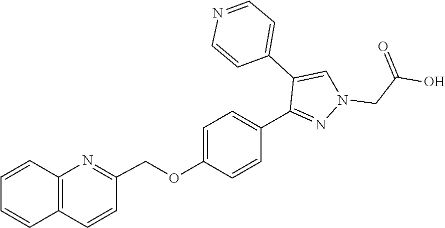 [SMILES: O=C(O)Cn1cc(-c2ccncc2)c(-c2ccc(OCc3ccc4ccccc4n3)cc2)n1]

1.0M NaOH (1.50 mL, 1.50 mmol) was added to a solution of intermediate 49 (134 mg, 48.6% pure, 140 μmol) in THF (1.5 mL) and MeCOH (0.5 mL) and stirred for 1 h. The mixture was neutralised with the addition of 1M HCl and concentrated in vacuo. The residue was purified by reverse phase HPLC to give the title compound (43.0 mg, 70.2%) as a yellow solid. UPLC (Method A) Rt 3.44 min, 99.9%. HRMS (ES+/QToF) m/z: [M+H]⁺ Calcd for C₂₆H₂₁N₄O₃ 437.1614; Found 437.1612.

### Example 6

### 2-[[4-[1-Methylsulfonyl-4-(4-pyridyl)pyrazol-3-yl]phenoxy]methyl]quinoline

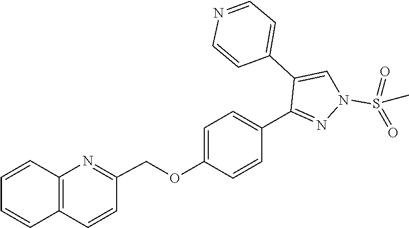 [SMILES: CS(=O)(=O)n1cc(-c2ccncc2)c(-c2ccc(OCc3ccc4ccccc4n3)cc2)n1]

Methanesulfonyl chloride (11.1 μL, 144 μmol) was added to a solution of intermediate 48 (50.0 mg, 98.9% pure, 131 μmol) and TEA (27.3 μL, 196 μmol) in DCM (5.0 mL) at 0° C. and the reaction stirred for 30 min. The reaction was then concentrated in vacuo and stirred in 10% MeCOH/Water (3.0 mL) for 10 min before the resultant solid was collected by filtration. The solid was then purified by reverse phase HPLC to give the title compound (8.50 mg, 13.6%) as a white solid. UPLC (Method A) Rt 3.92 min, 95.4%. HRMS (ES+/QToF) m/z: [M+H]⁺ Calcd for C₂₅H₂₁N₄O₃S 457.1334; Found 457.1331.

### Example 7

### N-[Dimethyl(oxo)-λ6-sulfanylidene]-5-methyl-2-[[4-[1-methyl-4-(4-pyridyl)pyrazol-3-yl]phenoxy]methyl]quinoline-3-carboxamide

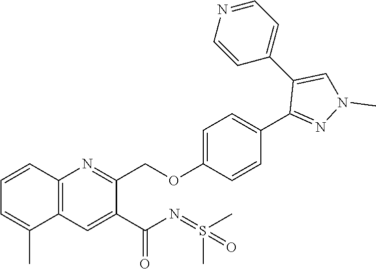 [SMILES: Cc1cccc2nc(COc3ccc(-c4nn(C)cc4-c4ccncc4)cc3)c(C(=O)N=S(C)(C)=O)cc12]

A mixture of intermediate 76 (20.0 mg, 0.04 mmol), iminodimethyl-λ6-sulfanone (8.00 mg, 0.08 mmol), DMAP (6.00 mg, 0.05 mmol), 2-chloro-1-methylpyridin-1-ium iodide (22.0 mg, 0.08 mmol) and TEA (13.0 mg, 0.13 mmol) in DCM was stirred overnight. The reaction mixture was concentrated in vacuo then purified by Prep-HPLC (NH₄HCO₃ buffered) to give the title compound (4.40 mg, 18.7%)* as a light yellow solid. UPLC (Method C) Rt 1.73 min, 99.7%. HRMS (ES+/QToF) m/z: [M+H]⁺ Calcd for C₂₉H₂₈N₅O₃S 526.1913; Found 526.1915.

### Example 8

### N-[Dimethyl(oxo)-λ6-sulfanylidene]-2-[[4-[4-(4-pyridyl)-1-(2,2,2-trifluoroethyl)pyrazol-3-yl]phenoxy]methyl]quinoline-4-carboxamide

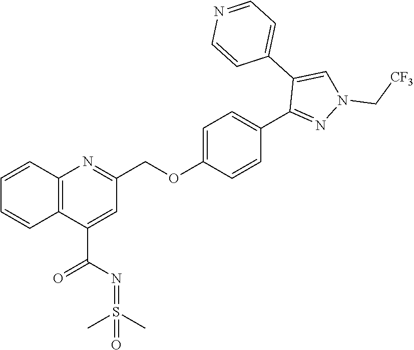 [SMILES: CS(C)(=O)=NC(=O)c1cc(COc2ccc(-c3nn(CC(F)(F)F)cc3-c3ccncc3)cc2)nc2ccccc12]

A mixture of intermediate 61 (45.0 mg, 0.09 mmol), iminodimethyl-λ6-sulfanone (16.6 mg, 0.18 mmol), DMAP (13.0 mg, 0.11 mmol), 2-chloro-1-methylpyridin-1-ium iodide (45.5 mg, 0.18 mmol), DIPEA (34.5 mg, 0.27 mmol) in DMF (5.0 mL) was stirred for 2 h. The reaction was quenched with water, extracted with EtOAc (2×5 mL), the combined organic layers washed with brine (2×5 mL), dried (Na₂SO₄) and concentrated in vacuo. The residue was purified by Prep-HPLC (NH₄HCO₃ buffered) to give the title compound (2.00 mg, 3.7%)* as a purple solid. UPLC (Method C) Rt 1.89 min, 97.3%. HRMS (ES+/QToF) m/z: [M+H]⁺ Calcd for C₂₉H₂₅N₅O₃F3S 580.1630; Found 580.1627.

### Example 9

### 2-[[4-[1-Methyl-4-(4-pyridyl)pyrazol-3-yl]phenoxy]methyl]-N-methylsulfonyl-quinoline-3-carboxamide

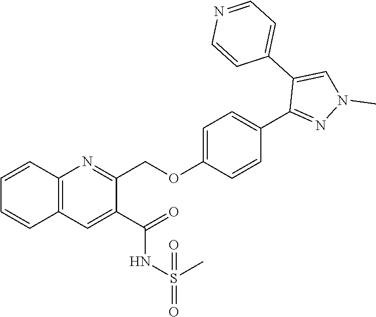 [SMILES: Cn1cc(-c2ccncc2)c(-c2ccc(OCc3nc4ccccc4cc3C(=O)NS(C)(=O)=O)cc2)n1]

DIPEA (58.7 μL, 337 μmol) was added to a suspension of example 3 (50.0 mg, 98.0% pure, 112 μmol), methanesulfonamide (21.4 mg, 224 μmol) and HATU (85.4 mg, 224 μmol) in DCM (1.2 mL) and stirred for 6.5 h. The reaction mixture was partitioned between DCM (30 mL) and H₂O (30 mL). The aq layer was extracted with DCM (30 mL) and the organic layers combined, dried (MgSO₄) and concentrated in vacuo. The residue was purified by reverse phase HPLC to give the title compound (18.8 mg, 32.1%) as a white solid. UPLC (Method A), Rt 3.96 min, 98.4%. HRMS (ES+/QToF) m/z: [M+H]⁺ Calcd for C₂₇H₂₄N₅O₄S 514.1549; Found 514.1542.

### Examples 10 to 14

Examples 10-14 were prepared similarly to example 9, by amide coupling of the appropriate intermediates with the appropriate amines using HATU; see Table 9 below.

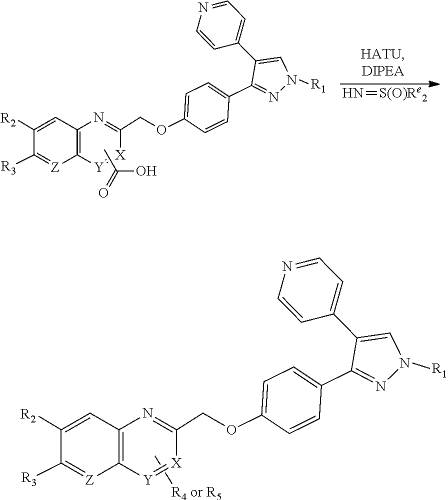

In the above scheme R⁴ and R⁵ can be —C(O)N═S(O)R^{e}₂.

TABLE 9
HATU couplings

| | | | Intermediate, Form, |
|---|---|---|---|
| Ex | Structure | Name | Yield, UPLC, HRMS |
| 10 | 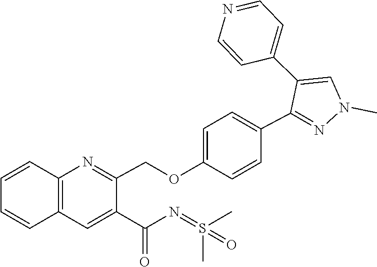 [SMILES: Cn1cc(-c2ccncc2)c(-c2ccc(OCc3nc4ccccc4cc3C(=O)N=S(C)(C)=O)cc2)n1] | N-[Dimethyl(oxo)- λ6-sulfanylidene]-2- [4-[1-methyl-4-(4- pyridyl)pyrazol-3- yl]phenoxy]methyl] quinoline-3- carboxamide | From example 3 White solid Yield 32.6 mg, 56.1% UPLC (Method A): Rt 3.71 min, 98.9% HRMS: Calcd for C₂₈H₂₆N₅O₃S 512.1756; Found 512.1758. |
| 11 | 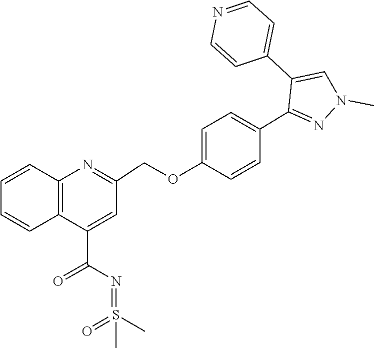 [SMILES: Cn1cc(-c2ccncc2)c(-c2ccc(OCc3cc(C(=O)N=S(C)(C)=O)c4ccccc4n3)cc2)n1] | N-[Dimethyl(oxo)- λ6-sulfanylidene]-2- [[4-[1-methyl-4-(4- pyridyl)pyrazol-3- yl]phenoxy]methyl] quinoline-4- carboxamide | From example 4 White solid Yield 165 mg, 40.6% UPLC (Method A): Rt 3.82 min, 100% HRMS: Calcd for C₂₈H₂₆N₅O₃S 512.1756; Found 512.1754. |
| 12 | 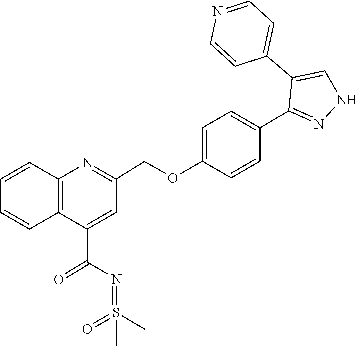 [SMILES: CS(C)(=O)=NC(=O)c1cc(COc2ccc(-c3n[nH]cc3-c3ccncc3)cc2)nc2ccccc12] | N-[Dimethyl(oxo)- λ6-sulfanylidene]-2- [[4-[4-(4-pyridyl)- 1H-pyrazol-3- yl]phenoxy]methyl] quinoline-4- carboxamide | From example 30 White solid Yield 42.8 mg, 27.6% UPLC (Method A): Rt 3.56 min, 98.9% HRMS: Calcd for C₂₇H₂₄N₅O₃S 498.1600; Found 498.1597. |
| 13 | 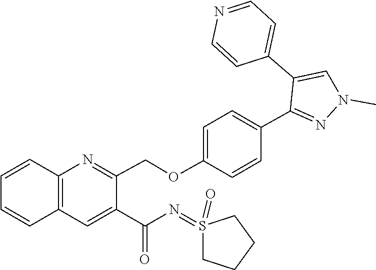 [SMILES: Cn1cc(-c2ccncc2)c(-c2ccc(OCc3nc4ccccc4cc3C(=O)N=S3(=O)CCCC3)cc2)n1] | 2-[[4-[1-Methyl-4-(4- pyridyl)pyrazol-3- yl]phenoxy]methyl]- N-(1-oxothiolan-1- ylidene)quinoline-3- carboxamide | From example 3 White solid Yield 91.4 mg, 75.1% UPLC (Method A): Rt 3.96 min, 100% HRMS: Calcd for C₃₀H₂₈N₅O₃S 538.1913; Found 538.1916. |
| 14 | 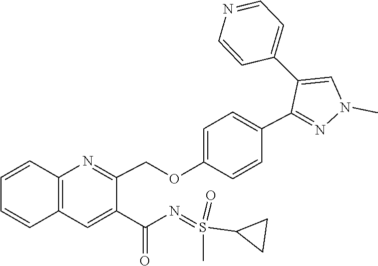 [SMILES: Cn1cc(-c2ccncc2)c(-c2ccc(OCc3nc4ccccc4cc3C(=O)N=S(C)(=O)C3CC3)cc2)n1] | N-(Cyclopropyl- methyl-oxo-λ6- sulfanylidene)-2-[[4- [1-methyl-4-(4- pyridyl)pyrazol-3- yl]phenoxy]methyl] quinoline-3- carboxamide | From example 3 White solid Yield 72.2 mg, 59.3% UPLC (Method A): Rt 3.96 min, 100% HRMS: Calcd for C₃₀H₂₈N₅O₃S 538.1913; Found 538.1913. |

### Example 15

### N-Methyl-2-[[4-[1-methyl-4-(4-pyridyl)pyrazol-3-yl]phenoxy]methyl]-N-methylsulfonyl-quinoline-3-carboxamide

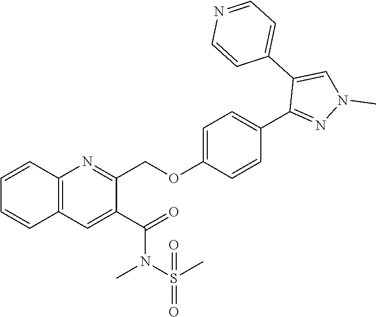 [SMILES: CN(C(=O)c1cc2ccccc2nc1COc1ccc(-c2nn(C)cc2-c2ccncc2)cc1)S(C)(=O)=O]

MeI (5.89 μL, 94.6 μmol) was added to the formic acid salt of example 9 (48.0 mg, 99.5% purity, 78.9 μmol) and K₂CO₃ (16.4 mg, 118 μmol) in acetone:DMF (2:1, 1.5 mL) and stirred for 42 h. The mixture was concentrated in vacuo then purified by reverse phase HPLC (NH3 buffered) to give the title compound (15.7 mg, 37.4%) as a white solid. UPLC (Method A) Rt 3.93 min, 99.1%. HRMS (ES+/QToF) m/z: [M+H]⁺ Calcd for C₂₈H₂₆N₅O₄S 528.1706; Found 528.1713.

### Example 16

### N-Methyl-2-[[4-[1-methyl-4-(4-pyridyl)pyrazol-3-yl]phenoxy]methyl]-N-methylsulfonyl-quinoline-4-carboxamide

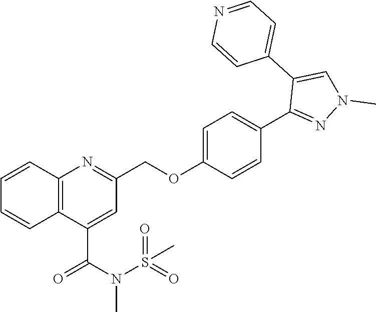 [SMILES: CN(C(=O)c1cc(COc2ccc(-c3nn(C)cc3-c3ccncc3)cc2)nc2ccccc12)S(C)(=O)=O]

DIPEA (0.10 mL, 0.57 mmol) was added to a solution of example 4 (102 mg, 98.2% pure, 0.23 mmol) and T3P (50 wt % solution in EtOAc, 0.28 mL, 0.47 mmol) in DMF (1.5 mL) and the mixture stirred for 5 min before N-methylmethane sulfonamide (40.0 μL, 0.47 mmol) was added. The reaction mixture was stirred at 40° C. for 3 h. The mixture was diluted with DCM (20 mL), washed with 1M aq NaOH (25 mL), dried (Na₂SO₄), and concentrated in vacuo. The residue was purified by reverse phase HPLC (NH₃ buffered) to give the title compound (19.7 mg, 16.3%) as a white solid. UPLC (Method A) Rt 4.26 min, 99.7%. HRMS (ES+/QToF) m/z: [M+H]⁺ Calcd for C₂₈H₂₆N₅O₄S 528.1706; Found 528.1705.

### Example 17

### N-[Dimethyl(oxo)-λ6-sulfanylidene]-2-[[4-[1-methyl-4-(4-pyridyl)pyrazol-3-yl]phenoxy]methyl]quinazoline-4-carboxamide

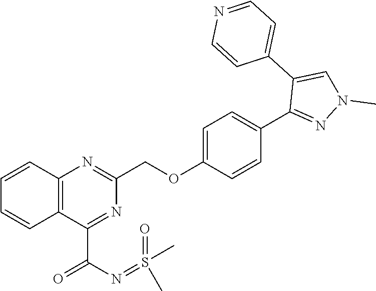 [SMILES: Cn1cc(-c2ccncc2)c(-c2ccc(OCc3nc(C(=O)N=S(C)(C)=O)c4ccccc4n3)cc2)n1]

Intermediate 65 (35.0 mg, 98.7% pure, 76.5 μmol) was dissolved in THF (2.0 mL) and water (2.0 mL). LiOH·H₂O (3.85 mg, 91.8 μmol) was added and the reaction was stirred for 1 h. The mixture was concentrated in vacuo. The residue was dissolved in DMF (2.0 mL), HATU (58.2 mg, 0.15 mmol), S,S-dimethyl sulfoximine (14.3 mg, 0.15 mmol) and DIPEA (26.7 μL, 0.15 mmol) were added and the reaction was stirred for 1.5 h. The mixture was diluted with DCM (30 mL), washed with sat aq NaHCO₃ (30 mL), dried (MgSO₄) and concentrated in vacuo. The residue was purified by reverse phase HPLC (NH₃ buffered) to give the title compound (24.4 mg, 61.8%) as a yellow solid. UPLC (Method A) Rt 3.68 min, 99.4%. HRMS (ES+/QToF) m/z: [M+H]⁺ Calcd for C₂₇H₂₅N₆O₃S 513.1709; Found 513.1710.

### Examples 18 to 20

Examples 18-20 were prepared similarly to example 17, by saponification of the appropriate intermediates followed by amide coupling with the appropriate amines using HATU; see Table 10 below.

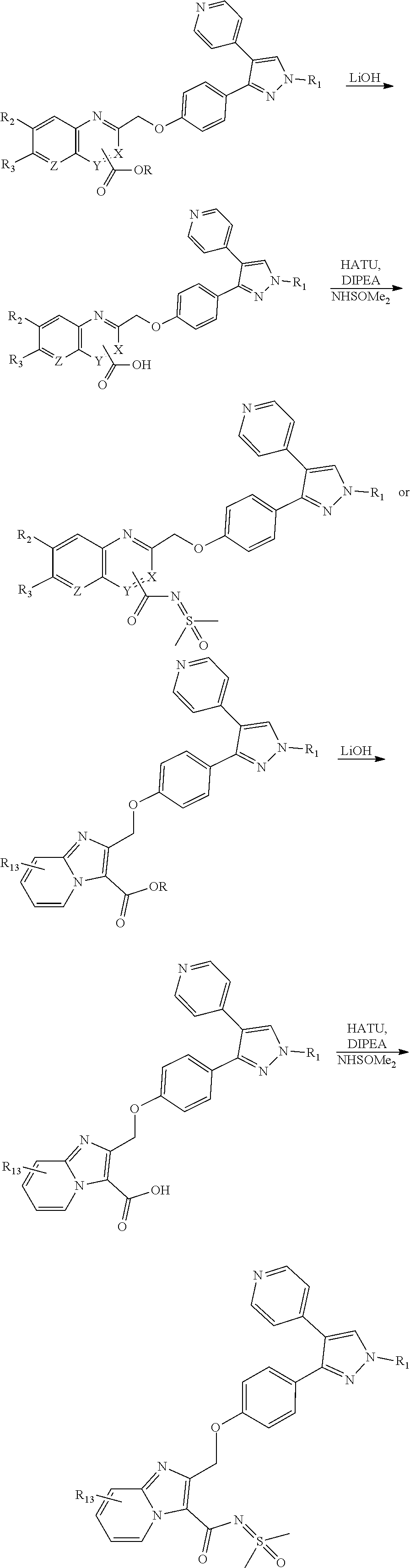

TABLE 10
Saponification followed by HATU couplings

| | | | Intermediate, Form, |
|---|---|---|---|
| Ex | Structure | Name | Yield, UPLC, HRMS |
| 18 | 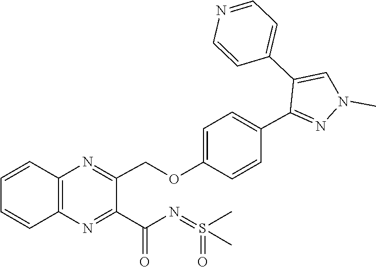 [SMILES: Cn1cc(-c2ccncc2)c(-c2ccc(OCc3nc4ccccc4nc3C(=O)N=S(C)(C)=O)cc2)n1] | N-[Dimethyl(oxo)- λ6-sulfanylidene]-3- [[4-[1-methyl-4-(4- pyridyl)pyrazol-3- yl]phenoxy]methyl] quinoxaline-2- carboxamide | From Intermediate 66 Yellow solid Yield 39.6 mg, 81.6% UPLC (Method A): Rt 3.78 min, 100% HRMS: Calcd for C₂₇H₂₅N₆O₃S 513.1709; Found 513.1708. |
| 19 | 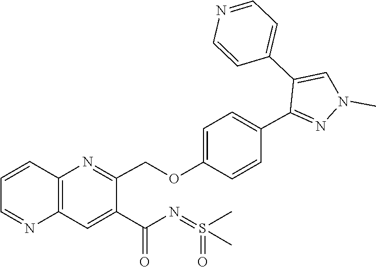 [SMILES: Cn1cc(-c2ccncc2)c(-c2ccc(OCc3nc4cccnc4cc3C(=O)N=S(C)(C)=O)cc2)n1] | N-[Dimethyl(oxo)- λ6-sulfanylidene]-2- [[4-[1-methyl-4-(4- pyridyl)pyrazol-3- yl]phenoxy]methyl]- 1,5-naphthyridine- 3-carboxamide | From Intermediate 67 Yellow solid Yield 49.9 mg, 53.8% UPLC (Method A): Rt 3.44 min, 100% HRMS: Calcd for C₂₇H₂₅N₆O₃S 513.1709; Found 513.1708. |
| 20 | 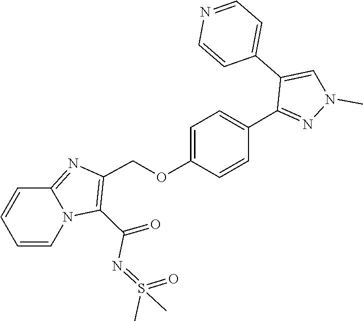 [SMILES: Cn1cc(-c2ccncc2)c(-c2ccc(OCc3nc4ccccn4c3C(=O)N=S(C)(C)=O)cc2)n1] | N-[Dimethyl(oxo)- λ6-sulfanylidene]-2- [[4-[1-methyl-4-(4- pyridyl)pyrazol-3- yl]phenoxy]methyl] imidazo[1,2- a]pyridine-3- carboxamide | From Intermediate 72 Pink solid Yield 19.4 mg, 22.5% UPLC (Method A) Rt 3.09 min, 98.7% HRMS: Calcd for C₂₆H₂₅N₆O₃S 501.1709; Found 501.1707. |

### Example 21

### N-[Dimethyl(oxo)-λ6-sulfanylidene]-7-fluoro-2-[[4-[l-methyl-4-(4-pyridyl)pyrazol-3-yl]phenoxy]methyl]quinoline-3-carboxamide

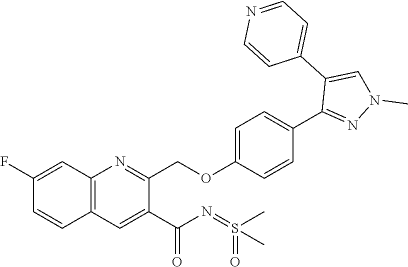 [SMILES: Cn1cc(-c2ccncc2)c(-c2ccc(OCc3nc4cc(F)ccc4cc3C(=O)N=S(C)(C)=O)cc2)n1]

Intermediate 52 (110 mg, 0.23 mmol) was dissolved in THF:H₂O (1:1), LiCH (10.9 mg, 0.46 mmol) was added and stirred for 12 h. The residue was acidified to pH 4 with aq HCl. The resulting mixture was extracted with EtOAc (3×5 mL), the combined organic layers washed with brine (3×3 mL), dried (Na₂SO₄) and concentrated in vacuo. The residue was dissolved in DCM (5.0 mL), S,S-dimethyl sulfoximine (6.20 mg, 0.07 mmol), 2-chloro-1-methylpyridin-1-ium iodide (16.9 mg, 0.07 mmol), DMAP (4.80 mg, 0.04 mmol) and DIPEA (12.8 mg, 0.10 mmol) were added and stirred for 2 h. The resulting mixture was extracted with DCM (3×5 mL), the combined organic layers were washed with water (3×5 mL), dried (Na₂SO₄) and concentrated in vacuo. The residue was purified by Prep-HPLC to give the title compound (2.80 mg, 16.0%)* as a white solid. UPLC (Method C) Rt 1.79 min, 96.5%. HRMS (ES+/QToF) m/z: [M+H]⁺ Calcd for C₂₈H₂₅N₅O₃FS 530.1662; Found 530.1657.

### Examples 22 to 28

Examples 22-28 were prepared similarly to example 21, by saponification of the appropriate intermediates followed by amide coupling with S,S-dimethyl sulfoximine using 2-chloro-1-methylpyridin-1-ium iodide; see Table 11 below.

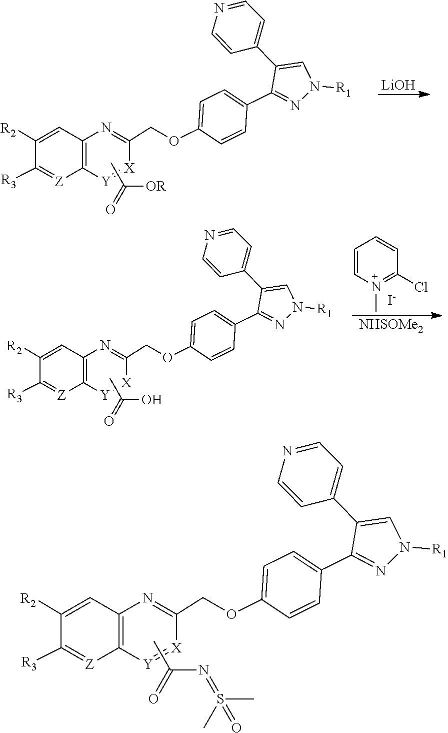

In the above scheme, group “OR” can be O-alkyl, such as —OMe or -OEt.

TABLE 11
Saponification followed by amide couplings using 2-chloro-1-
methylpyridin-1-ium iodide

| | | | Intermediate, Form, |
|---|---|---|---|
| Ex | Structure | Name | Yield, UPLC, HRMS |
| 22 | 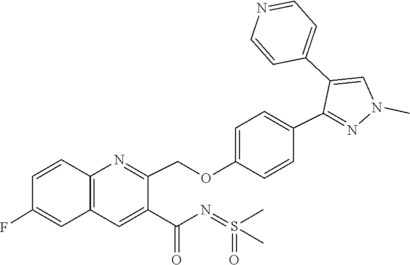 [SMILES: Cn1cc(-c2ccncc2)c(-c2ccc(OCc3nc4ccc(F)cc4cc3C(=O)N=S(C)(C)=O)cc2)n1] | N-[Dimethyl(oxo)-λ6- sulfanylidene]-6- fluoro-2-[[4-[1- methyl-4-(4- pyridyl)pyrazol-3- yl]phenoxy]methyl] quinoline-3- carboxamide | From Intermediate 53 White solid Yield 62.9 mg, 28.7%* UPLC (Method C) Rt 1.74 min, 99.6% HRMS: Calcd for C₂₈H₂₅N₅O₃FS 530.1662; Found 530.1662. |
| 23 | 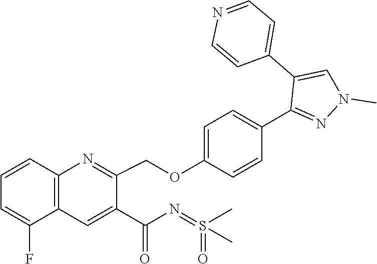 [SMILES: Cn1cc(-c2ccncc2)c(-c2ccc(OCc3nc4cccc(F)c4cc3C(=O)N=S(C)(C)=O)cc2)n1] | N-[Dimethyl(oxo)-λ6- sulfanylidene]-5- fluoro-2-[[4-[1- methyl-4-(4- pyridyl)pyrazol-3- yl]phenoxy]methyl] quinoline-3- carboxamide | From Intermediate 54 White solid Yield 26.5 mg, 17.5%* UPLC (Method C) Rt 1.73 min, 99.2% HRMS: Calcd for C₂₈H₂₅N₅O₃FS 530.1662; Found 530.1659. |
| 24 | 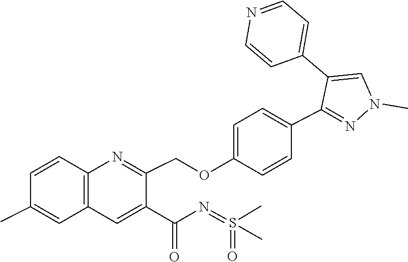 | N-[Dimethyl(oxo)-λ6- sulfanylidene]-6- methyl-2-[[4-[1- methyl-4-(4- pyridyl)pyrazol-3- yl]phenoxy]methyl] quinoline-3- carboxamide | From Intermediate 75 Yellow oil Yield 1.6 mg, 4.57%* UPLC (Method C) Rt 1.79 min, 68.6%. HRMS: Calcd for C₂₈H₂₅N₅O₃FS 530.1662; Found 530.1659. |
| 25 | 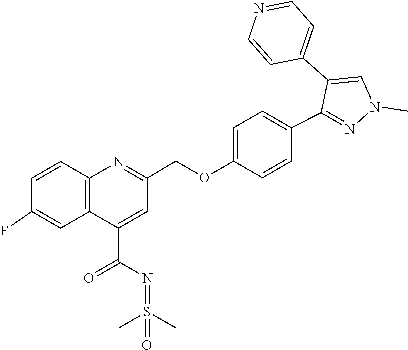 [SMILES: Cn1cc(-c2ccncc2)c(-c2ccc(OCc3cc(C(=O)N=S(C)(C)=O)c4cc(F)ccc4n3)cc2)n1] | N-[Dimethyl(oxo)-λ6- sulfanylidene]-6- fluoro-2-[[4-[1- methyl-4-(4- pyridyl)pyrazol-3- yl]phenoxy]methyl] quinoline-4- carboxamide | From Intermediate 57 White solid Yield 15.0 mg, 14.3%* UPLC (Method C) Rt 1.75 min, 98.7%. HRMS: Calcd for C₂₈H₂₅N₅O₃FS 530.1662; Found 530.1663. |
| 26 | 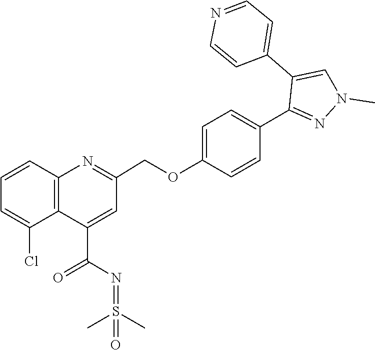 [SMILES: Cn1cc(-c2ccncc2)c(-c2ccc(OCc3cc(C(=O)N=S(C)(C)=O)c4c(Cl)cccc4n3)cc2)n1] | 5-Chloro-N- [Dimethyl(oxo)-λ6- sulfanylidene]-2-[[4- [1-methyl-4-(4- pyridyl)pyrazol-3- yl]phenoxy]methyl] quinoline-4- carboxamide | From Intermediate 58 Off white solid Yield 1.2 mg, 6.47%* UPLC (Method C) Rt 1.71 min, 91.5%. HRMS: Calcd for C₂₈H₂₅N₅O₃SCl 546.1367; Found 546.1368. |
| 27 | 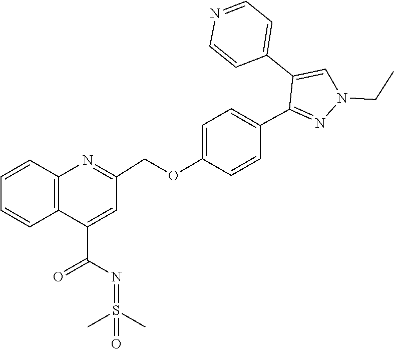 [SMILES: CCn1cc(-c2ccncc2)c(-c2ccc(OCc3cc(C(=O)N=S(C)(C)=O)c4ccccc4n3)cc2)n1] | N-[Dimethyl(oxo)-λ6- sulfanylidene]-2-[[4- [1-ethyl-4-(4- pyridyl)pyrazol-3- yl]phenoxy]methyl] quinoline-4- carboxamide | From Intermediate 59 White solid Yield 2.8 mg, 8.0%* UPLC (Method C) Rt 1.76 min, 99.4%. HRMS: Calcd for C₂₉H₂₈N₅O₃S 526.1913; Found 526.1912. |
| 28 | 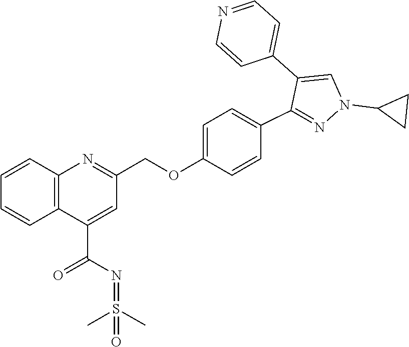 [SMILES: CS(C)(=O)=NC(=O)c1cc(COc2ccc(-c3nn(C4CC4)cc3-c3ccncc3)cc2)nc2ccccc12] | 2-[[4-[1-Cyclopropyl- 4-(4-pyridyl)pyrazol- 3- yl]phenoxy]methyl]- N-[dimethyl(oxo)-λ6- sulfanylidene] quinoline-4- carboxamide | From Intermediate 60 Yellow solid Yield 4.2 mg, 35.0%* UPLC (Method C) Rt 1.80 min, 99.0%. HRMS: Calcd for C₃₀H₂₈N₅O₃S 538.1913; Found 538.1916. |

### Example 29

### 2-[[4-[4-(4-Pyridyl)-1H-pyrazol-3-yl]phenoxy]methyl]quinoline-3-carboxylic acid

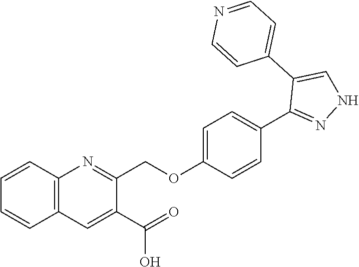 [SMILES: O=C(O)c1cc2ccccc2nc1COc1ccc(-c2n[nH]cc2-c2ccncc2)cc1]

HCl (4M in 1,4-dioxane) (3.00 mL, 12.0 mmol) was added to a solution of intermediate 69 (70.0 mg, 88.6% pure, 116 μmol) in 1,4-dioxane (3.0 mL) and the reaction was stirred for 16 h. The reaction mixture was concentrated in vacuo. The residue was dissolved in THF (1.0 mL) and water (1.0 mL), LiOH·H₂O (24.2 mg, 578 μmol) was added and the reaction was stirred for 2 h. The reaction mixture was neutralised to pH 7 by addition of 1.0M aq HCl and concentrated in vacuo. The residue was purified by reverse phase HPLC to give the title compound (16.1 mg, 32.9%) as a white solid. UPLC (Method A) Rt 3.58 min, 99.6%. HRMS (ES+/QToF) m/z: [M+H]⁺ Calcd for C₂₅H₁₉N₄O₃FS 423.1457; Found 423.1455.

### Example 30

### 2-[[4-[4-(4-Pyridyl)-1H-pyrazol-3-yl]phenoxy]methyl]quinoline-4-carboxylic acid

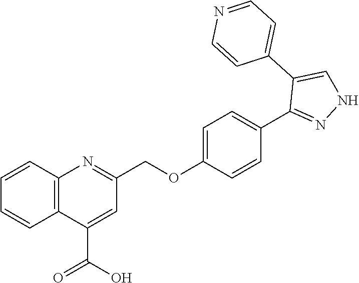 [SMILES: O=C(O)c1cc(COc2ccc(-c3n[nH]cc3-c3ccncc3)cc2)nc2ccccc12]

LiOH·H₂O (31.4 mg, 749 μmol) was added to a solution of intermediate 70 (153 mg, 87.5% pure, 250 μmol) in THF (2.5 mL) and water (2.5 mL) and the reaction was stirred for 16 h. The THF was removed in vacuo. The remaining aq portion was neutralised to pH 7 with 1M aq HCl and the resulting precipitate was collected by filtration. The product was purified by reverse phase HPLC to give the title compound (8.27 mg, 7.76%) as a white solid. UPLC (Method A) Rt 3.48 min, 98.9%. HRMS (ES+/QToF) m/z: [M+H]⁺ Calcd for C₂₅H₁₉N₄O₃ 423.1457; Found 423.1459.

### Example 31

### N-[Dimethyl(oxo)-λ6-sulfanylidene]-2-[[4-[4-(4-pyridyl)-1H-pyrazol-3-yl]phenoxy]methyl]quinoline-3-carboxamide

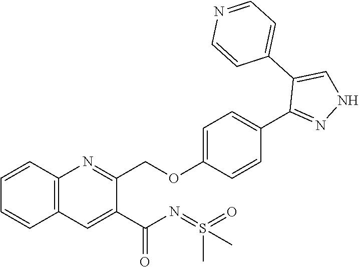 [SMILES: CS(C)(=O)=NC(=O)c1cc2ccccc2nc1COc1ccc(-c2n[nH]cc2-c2ccncc2)cc1]

Intermediate 81 (20.0 mg) was dissolved in DCM (1.0 mL) and TFA (0.2 mL) and stirred for 45 min. The reaction was concentrated in vacuo then purified by Prep-HPLC (TFA buffered) to give the title compound (7.90 mg, 49.8%)* as a yellow solid. LCMS (ES+): 498.2 [MH]⁺. UPLC (Method C) Rt 1.60 min, 88.3%. HRMS (ES+/QToF) m/z: [M+H]⁺ Calcd for C₂₇H₂₄N₅O₃S 498.1600; Found 498.1604.

### Example 32

### Dimethyl-oxo-[[2-[[4-[4-(4-pyridyl)-1H-pyrazol-3-yl]phenoxy]methyl]-4-quinolyl]imino]-λ6-sulfane

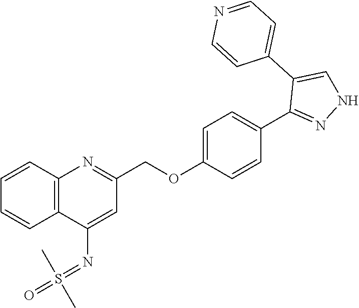 [SMILES: CS(C)(=O)=Nc1cc(COc2ccc(-c3n[nH]cc3-c3ccncc3)cc2)nc2ccccc12]

A mixture of Intermediate 71 (155 mg, 93.1% pure, 259 μmol), S,S-dimethyl sulfoximine (96.5 mg, 1.04 mmol), Pd(OAc)₂ (2.91 mg, 12.9 μmol), BINAP (24.2 mg, 38.8 μmol) and Cs₂CO₃ (169 mg, 518 μmol) in toluene (2.6 mL) was sparged with N₂ for 5 min. The reaction was heated at reflux for 18 h then in a microwave reactor at 120° C. for 30 min. MeCOH (10 mL) was added, and the mixture was filtered, concentrated in vacuo then purified by normal phase column chromatography and by reverse phase HPLC (ammonia buffered) to give the title compound (1.92 mg, 1.57%) as a yellow solid. UPLC (Method A) Rt 3.31 min, 99.3%. HRMS (ES+/QToF) m/z: [M+H]⁺ Calcd for C₂₆H₂₄N₅O₂S 470.1651; Found 470.1650.

### Example 33

### Imino-methyl-oxo-[2-[[4-[4-(4-pyridyl)-1H-pyrazol-3-yl]phenoxy]methyl]-4-quinolyl]-λ6-sulfane

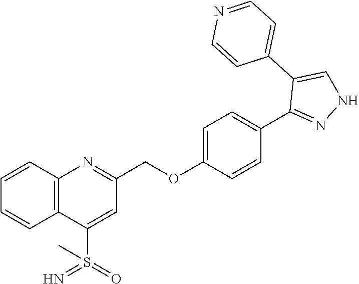 [SMILES: CS(=N)(=O)c1cc(COc2ccc(-c3n[nH]cc3-c3ccncc3)cc2)nc2ccccc12]

A mixture of intermediate 79 (91.0 mg, 61.3% pure, 127 μmol), ammonium acetate (117 mg, 1.52 mmol) and iodobenzene diacetate (367 mg, 1.14 mmol) in MeCOH (1.0 mL) was stirred for 48 h. Ammonium acetate (39.0 mg, 507 umol) and iodobenzene diacetate (122 mg, 380 umol) were added and the reaction was stirred for 24 h. DCM (10 mL) and sat aq NaHCO₃ (10 mL) were added. The organic and aq layers were decanted off then purified by normal phase column chromatography and by reverse phase HPLC (ammonia buffered) to give the title compound (3.52 mg, 6.02%) as a yellow solid. UPLC (Method A) Rt 3.48 min, 98.6%, HRMS (ES+/QToF) m/z: [M+H]⁺ Calcd for C₂₅H₂₂N₅O₂S 456.1494; Found 456.1492.

### Example 34

### N-[2-[[4-[1-Methyl-4-(4-pyridyl)pyrazol-3-yl]phenoxy]methyl]-4-quinolyl]methanesulfonamide

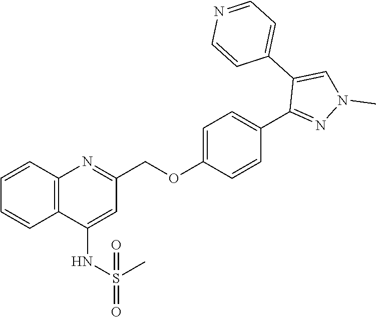 [SMILES: Cn1cc(-c2ccncc2)c(-c2ccc(OCc3cc(NS(C)(=O)=O)c4ccccc4n3)cc2)n1]

Under N₂ a mixture of intermediate 68 (113 mg, 96.0% pure, 230 umol), Pd₂(dba)₃ (12.6 mg, 13.8 μmol), tBuXPhos (17.6 mg, 41.1 μmol), K₂CO₃ (63.6 mg, 460 umol) and methanesulfonamide (21.9 mg, 230 umol) in 1,4-dioxane (1.2 mL) was sparged with N2 for 5 min. The reaction was heated at 60-90° C. for 19 h. The reaction mixture was filtered through Celite and concentrated in vacuo then purified by reverse phase HPLC to give the title compound (28.8 mg, 25.6%) as a white solid. UPLC (Method A), Rt 3.62 min, 99.3%. HRMS (ES+/QToF) m/z: [M+H]⁺ Calcd for C₂₆H₂₄N₅O₂S 486.1600; Found 486.1603.

### Example 35

### 1-Methyl-3-[2-[[4-[1-methyl-4-(4-pyridyl)pyrazol-3-yl]phenoxy]methyl]-4-quinolyl]urea

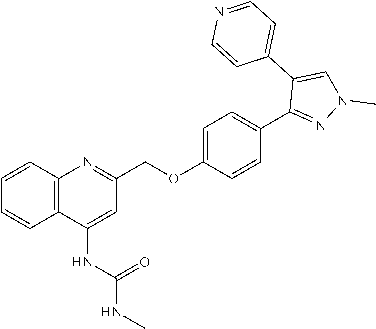 [SMILES: CNC(=O)Nc1cc(COc2ccc(-c3nn(C)cc3-c3ccncc3)cc2)nc2ccccc12]

A mixture of intermediate 68 (113 mg, 96.0% pure, 230 μmol)N-methylurea (85.3 mg, 1.15 mmol) Pd₂(dba)₃ (21.1 mg, 23.0 μmol), xantphos (26.6 mg, 46.0 μmol), CuI (13.1 mg, 69.0 μmol) and NaOtBu (111 mg, 1.15 mmol) in 1,4-dioxane (2.0 mL) was sparged with N₂ for 5 min. The reaction was heated at 110° C. for 30 min in a microwave reactor. The reaction mixture was diluted with MeCOH (10 mL) and filtered through celite and the filtrate was concentrated in vacuo. The residue was purified by reverse phase column chromatography and by reverse phase HPLC (formic acid buffered). The fractions were pooled, neutralised with sat aq NaHCO₃ (25 mL), extracted with DCM (2×25 mL), dried (MgSO₄) and concentrated in vacuo to give the title compound (16.5 mg, 15.4%) as a white solid. UPLC (Method A) Rt 3.33 min, 99.5%. HRMS (ES+/QToF) m/z: [M+H]⁺ Calcd for C₂₇H₂₅N₆O₂ 465.2039; Found 465.2042.

### Example 36

### 1-[Dimethyl(oxo)-λ6-sulfanylidene]-3-[2-[[4-[1-methyl-4-(4-pyridyl)pyrazol-3-yl]phenoxy]methyl]-4-quinolyl]urea

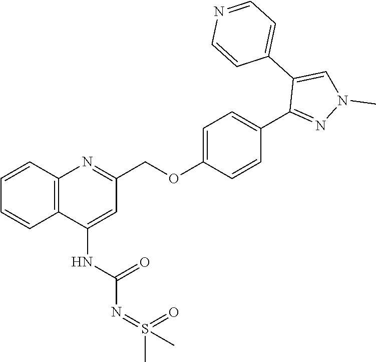 [SMILES: Cn1cc(-c2ccncc2)c(-c2ccc(OCc3cc(NC(=O)N=S(C)(C)=O)c4ccccc4n3)cc2)n1]

To a stirred solution of intermediate 80 (70.0 mg, 0.12 mmol) in THF (1.0 mL) was added HCl (2M, 1.0 mL) and stirred for 20 min. The reaction was concentrated in vacuo. The residue was dissolved in DCM, DIPEA (108 mg, 0.83 mmol) and triphosgene (12.4 mg, 0.04 mmol) were added dropwise at 0° C. under N₂ then stirred for at 0° C. for 30 min. Iminodimethyl-λ6-sulfanone (15.5 mg, 0.17 mmol) was added and stirred for 1 h. The reaction was concentrated in vacuo then purified by prep-HPLC (NH₄HCO₃ buffered) to give the title compound (2.80 mg, 6.37%)* as a white solid. UPLC (Method C) Rt 1.41 min, 96.1%. HRMS (ES+/QToF) m/z: [M+H]⁺ Calcd for C₂₆H₂₇N₆O₂S 527.1865; Found 527.1868.

### Example 37

### 4-[2-[[4-[1-Methyl-4-(4-pyridyl)pyrazol-3-yl]phenoxy]methyl]quinazolin-4-yl]-1,4-thiazinane 1,1-dioxide

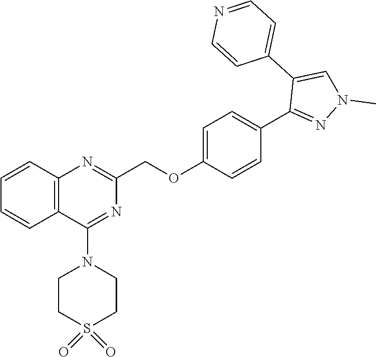 [SMILES: Cn1cc(-c2ccncc2)c(-c2ccc(OCc3nc(N4CCS(=O)(=O)CC4)c4ccccc4n3)cc2)n1]

To a solution of intermediate 11 (50.0 mg, 0.20 mmol) in dry DMF (8.0 mL) was added NaH (60% in oil, 16.0 mg) at 0° C. and stirred for 30 min. Intermediate 43 (93.0 mg, 0.30 mmol) was added and the mixture was allowed to warm to room temperature for 1 h. The reaction mixture was quenched by water and extracted with EtAOc (3×25 mL), the combined organic layers were washed with water (3×10 mL), dried (Na₂SO₄) and concentrated in vacuo. The residue was purified by prep-HPLC to give the title compound (36.0 mg, 34.4%)* as a white solid. UPLC (Method C) Rt 1.49 min, 99.0%. HRMS (ES+/QToF) m/z: [M+H]⁺ Calcd for C₂₈H₂₇N₆O₂S 527.1865; Found 527.1863.

### Example 38

### 4-[2-[[4-[1-Methyl-4-(4-pyridyl)pyrazol-3-yl]phenoxy]methyl]quinazolin-4-yl]piperazin-2-one

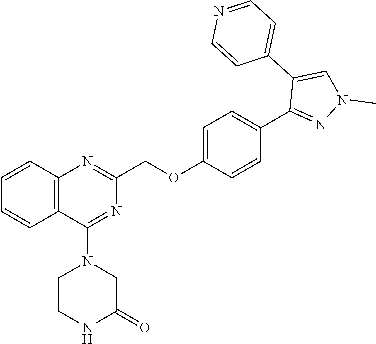 [SMILES: Cn1cc(-c2ccncc2)c(-c2ccc(OCc3nc(N4CCNC(=O)C4)c4ccccc4n3)cc2)n1]

A mixture of intermediate 64 (30.0 mg, 0.07 mmol), piperazin-2-one (11.0 mg, 0.11 mmol), BOP (38.9 mg, 0.09 mmol) and 1,8-diazabicyclo(5.4.0)undec-7-ene (33.5 mg, 0.21 mmol) in DMF was stirred for 2 h. The resulting mixture was quenched with water (5.0 mL), extracted with EtOAc (2×5.0 mL), the combined organic layers were washed with water (2×5.0 mL), dried (Na₂SO₄) then concentrated in vacuo. The residue was purified by silica gel column chromatography and by prep-HPLC (NH₄HCO₃ buffered) to give the title compound (6.30 mg, 17.5%)* as a white solid. UPLC (Method C) Rt 1.32 min, 99.3%. HRMS (ES+/QToF) m/z: [M+H]⁺ Calcd for C₂₆H₂₆N₇O₂ 492.2148; Found 492.2147.

### Example 39

### 1-[2-[[4-[1-Methyl-4-(4-pyridyl)pyrazol-3-yl]phenoxy]methyl]quinazolin-4-yl]azetidin-3-amine

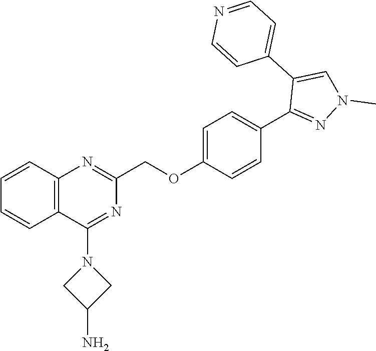 [SMILES: Cn1cc(-c2ccncc2)c(-c2ccc(OCc3nc(N4CC(N)C4)c4ccccc4n3)cc2)n1]

A solution of intermediate 73 (50.0 mg, 0.09 mmol) and TFA (2.0 mL) in DCM (4.0 mL) was stirred for 1 h. The reaction was concentrated in vacuo then purified by prep-HPLC (NH₄HCO₃ buffered) to give the title compound (6.30 mg, 15.1%)* as a yellow solid. UPLC (Method C) Rt 1.10 min, 89.0%. HRMS (ES+/QToF) m/z: [M+H]⁺ Calcd for C₂₇H₂₆N₇O 464.2199; Found 464.2199. EXAMPLES 40 to 42 Examples 40-42 were prepared similarly to example 39, by Boc deprotection of the appropriate intermediates with TFA; see Table 12 below.

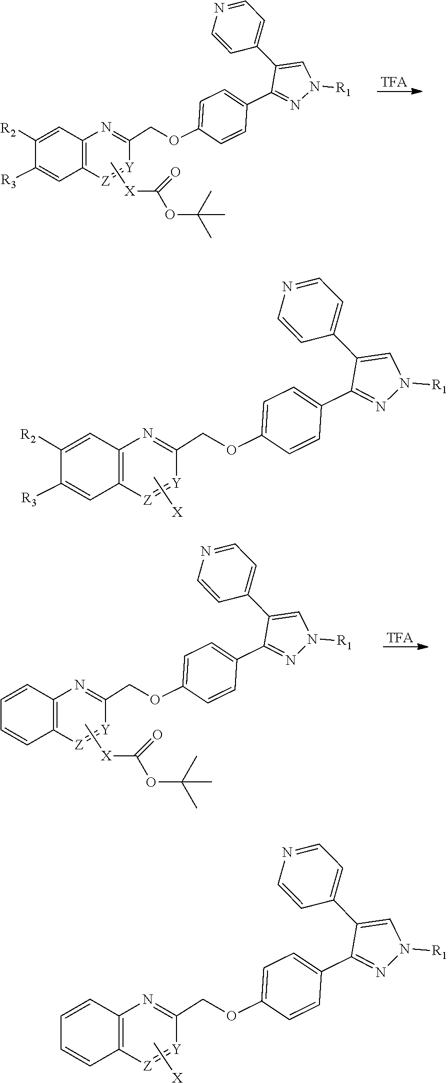

In the above scheme, X is

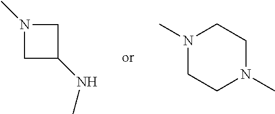

TABLE 12
TFA deprotections

| | | | Intermediate, Form, |
|---|---|---|---|
| Ex | Structure | Name | Yield, UPLC, HRMS |
| 40 | 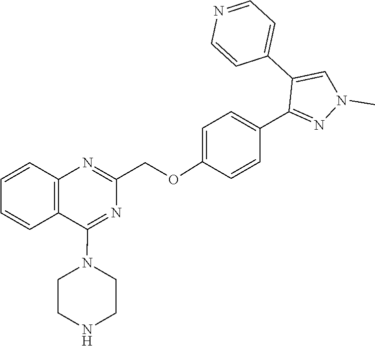 [SMILES: Cn1cc(-c2ccncc2)c(-c2ccc(OCc3nc(N4CCNCC4)c4ccccc4n3)cc2)n1] | 2-[[4-[1-Methyl-4-(4- pyridyl)pyrazol-3- yl]phenoxy]methyl]- 4-piperazin-1-yl- quinazoline | From Intermediate 74 White solid Yield 4.20 mg, 24.2%* UPLC (Method C) Rt 1.24 min, 94.3%. HRMS: Calcd for C₂₈H₂₈N₇O 478.2355; Found 478.2352. |
| 41 | 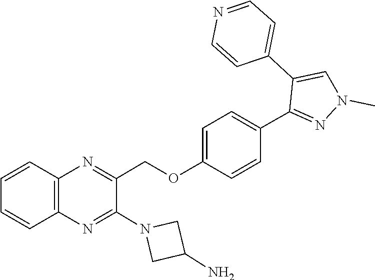 [SMILES: Cn1cc(-c2ccncc2)c(-c2ccc(OCc3nc4ccccc4nc3N3CC(N)C3)cc2)n1] | 1-[3-[[4-[1-Methyl-4- (4-pyridyl)pyrazol- 3- yl]phenoxy]methyl] quinoxalin-2- yl]azetidin-3-amine | From Intermediate 77 Yellow solid Yield 3.90 mg, 15.0%* UPLC (Method C) Rt 1.43 min, 93.5%. HRMS: Calcd for C₂₇H₂₆N₇O 464.2199; Found 464.2196. |
| 42 | 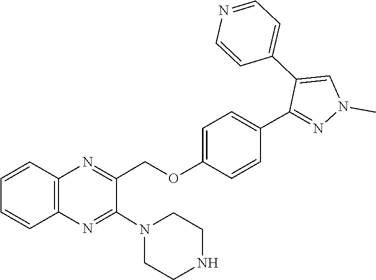 [SMILES: Cn1cc(-c2ccncc2)c(-c2ccc(OCc3nc4ccccc4nc3N3CCNCC3)cc2)n1] | 2-[[4-[1-Methyl-4-(4- pyridyl)pyrazol-3- yl]phenoxy]methyl]- 3-piperazin-1-yl- quinoxaline | From Intermediate 63 Yellow solid Yield 6.80 mg, 26.8%* UPLC (Method C) Rt 1.45 min, 98.2%. HRMS: Calcd for C₂₈H₂₈N₇O 478.2355; Found 478.2354. |

### Biochemical Human PDE10A Activity Assay—PDE10A2 Phosphate Sensor Assay

Semi Log compound dilutions starting at a final concentration of 50 μM were dispensed into a black 384 well plate alongside, DMSO and an Inhibited control using the Echo Acoustic dispenser. Both Human PDE10A2 and CD73 in a tris-based assay buffer at the final assay concentrations of 0.25 nM and 1 nM respectively, were pre incubated with the compounds for 15 minutes at room temperature, prior to the addition of the substrates, cGMP, and Phosphate Sensor, also diluted in a tris-based assay buffer at the final assay concentrations of 3 μM and 0.9 μM, respectively. The plate was incubated for a further 35 minutes at room temperature before the fluorescence intensity was measured using an optical filter of Ex 430 nm/Em 450 nm on the BMG CLARIOStar Plate Reader. Data was analysed using a 4-parameter fit.

Cell Based Human PDE10A Activity Assay—cAMP HTRF Assay in HEK 293 rhPDE10A2 Cell Line

Semi log compound dilutions starting at a final concentration of 10 μM were dispensed into a white 384 well plate, alongside DMSO and an Inhibited control using the Tecan D300e digital dispenser. HEK 293 cells overexpressing recombinant human PDE10A2 were seeded on top of the compounds at 2500 cells/well in a volume 5 μL/well. The plate was incubated at room temperature for 60 minutes. To induce endogenous cAMP, 5 μL/well of Forskolin at a final assay concentration of 10 μM was added to the plate. The plate was incubated at room temperature for a further 45 minutes. cAMP HTRF detection reagents were added, and the plate was incubated for 60 minutes at room temperature. The FRET signal was measured using an HTRF optical filter (337/620/665) on the BMG PHERAstar FS Plate Reader. Data was analysed using a 4-parameter fit.

TABLE 13
PDE10 inhibition data

| | | cAMP HTRF Assay |
|---|---|---|
| | PDE10A2 Phosphate | in HEK 293 |
| | Sensor Enzyme Assay | rhPDE10A2 cell line |
| Example | (pIC₅₀) | (pIC₅₀) |
| 1 | 9.43 | 8.71 |
| 2 | 9.69 | 9.93 |
| 3 | 10.1 | 8.56 |
| 4 | 10.1 | 9.09 |
| 5 | 9.41 | 7.12 |
| 6 | 8.36 | 7.19 |
| 7 | 9.65 | 8.83 |
| 8 | 10.2 | 9.42 |
| 9 | 10.1 | 8.78 |
| 10 | 10 | 8.96 |
| 11 | 10.1 | 9.00 |
| 12 | 9.84 | 8.30 |
| 13 | 9.86 | 9.01 |
| 14 | 10.0 | 9.17 |
| 15 | 9.22 | 7.97 |
| 16 | 9.87 | 9.22 |
| 17 | 9.92 | 8.52 |
| 18 | 9.34 | 6.99 |
| 19 | 9.43 | 8.27 |
| 20 | 9.76 | 8.17 |
| 21 | 8.32 | 6.26 |
| 22 | 9.49 | 7.76 |
| 23 | 9.59 | 8.40 |
| 24 | 8.29 | 6.48 |
| 25 | 9.41 | 8.44 |
| 26 | 9.13 | 7.37 |
| 27 | 10.2 | 9.27 |
| 28 | 9.42 | 8.56 |
| 29 | 10 | 9.46 |
| 30 | 10.2 | 9.86 |
| 31 | 10 | 7.86 |
| 32 | 10.3 | 7.33 |
| 33 | 9.32 | 8.05 |
| 34 | 9.54 | 8.92 |
| 35 | 9.67 | 8.46 |
| 36 | 9.46 | 7.78 |
| 37 | 9.65 | 8.83 |
| 38 | 10.1 | 9.57 |
| 39 | 9.46 | 7.61 |
| 40 | 8.98 | 8.34 |
| 41 | 9.45 | 8.12 |
| 42 | 8.96 | 7.91 |

The data in the table above show that the compounds of the invention, compounds of formulae (IA), (IB), (IIA), and (IIB), are potent PDE10A inhibitors, and may therefore be suitable for use in the treatment of inflammatory bowel diseases, such as ulcerative colitis and/or Crohn's disease.

### Determination of CNS Penetration In Vivo

Male Sprague Dawley Rats 300-350 g (Charles River, UK) were group housed, n=3, under a 12 hour light/dark cycle with food and water available ad libitum. Two days prior to dosing, animals were anaesthetised with inhaled isoflurane, and the right jugular vein was exposed and surgically cannulated. Animals were then housed singly for recovery, and throughout the remaining procedure. On the day of dosing animals were weighed, tail marked and dosed intravenously via the indwelling cannula with compound at 1 mg/kg in a volume of 3-5 mL/kg. Animals were culled at 10-30 min post dose via intravenous administration of pentobarbital. Post mortem blood was withdrawn via cardiac puncture, and briefly stored in K2 EDTA blood tubes on ice before being spun at 14,000 g for 4 min at 4° C. Plasma was withdrawn into a 96 well plate, placed on dry ice and stored at −80° C. Brains were quickly dissected and placed on dry ice before storage at −80° C.

Following dosing of test compound (intravenous) to Male Sprague-Dawley Rats, animals are sacrificed at one timepoint. Plasma is isolated from whole blood following cardiac exsanguination by centrifugal blood fractionation and whole brains isolated. Samples are stored on-ice and transferred to the Bioanalytical lab storage at −80° C. Bioanalysis of plasma and brain samples is performed as detailed below.

### Plasma Bioanalysis

Typically, a 1.00 mg/mL DMSO stock was used to prepare calibration standards of test compound in the range 1.00-6,000 ng/mL. Calibration lines were prepared by printing known masses of analyte into a 96-well plate in the range 25 to 150,000 pg. A volume of 25 μL of control male Sprague-Dawley Rat plasma was added to each well to prepare calibration standards at the appropriate concentration across the calibration range. Experimental samples were thawed to room temperature and 25 μL aliquots were added to the 96-well precipitation plate alongside the calibration lines. Samples were extracted using protein precipitation (agitation for at least 5 min at RT with 400 μL of MeCN containing 25 ng/mL tolbutamide as an internal standard). Protein precipitates were separated from the extracted test compound by centrifugation at 4000 rpm for 5 min, 4° C. The resulting supernatants were diluted in a ratio of 1:2 with diluent, 1:1 MeOH:H₂O.

Samples were analysed by UPLC-MS/MS on either an AB Sciex AP16500 QTrap or Waters TQ-S mass spectrometer using previously optimised analytical MRM (multiple reaction monitoring) methods, specific to the test compound.

The concentration of test compound in isolated samples was determined following analysis of the samples against the two replicates of the calibration line, injected before and after the sample set with an appropriate regression and weighting used. Only calibrators within ±15% of the expected test concentration value were included in the calibration line (±20% at the LLoQ) and any samples that fell outside of the limits of the calibration line were deemed to be less than or above the limit of quantification (LLoQ/ALoQ).

### Brain Bioanalysis

Typically, a 1.00 mg/mL DMSO stock was used to prepare calibration standards of test compound in the range 3.00-18,000 ng/mL. Calibration lines were prepared by printing known masses of analyte into a 96-well plate in the range 25 to 150,000 pg. A volume of 25 μL of control male Sprague-Dawley Rat brain homogenate (containing 8.33 mg of brain tissue) was added to each well to prepare calibration standards at the appropriate concentration across the calibration range.

To prepare control and experimental brain homogenates, brains were thawed at room temperature, weighed and a volume of diluent added (50:50 MeCN/H₂O) in the ratio of 2 mL per gram of brain. Homogenisation of brains was performed by bead-beater homogenisation using Precellys Evolution and CKMix50 7 mL mixed ceramic bead homogenisation tubes.

Aliquots of 25 μL experimental sample were extracted alongside the calibration lines using protein precipitation (agitation for at least 5 min at room temperature with 400 μL of MeCN containing 25 ng/mL tolbutamide as an internal standard). Protein precipitates were separated from the extracted test compound by centrifugation at 4000 rpm for 5 min, 4° C. The resulting supernatants were diluted in a ratio of 1:2 with diluent, 1:1 MeOH:H₂O.

Samples were analysed by UPLC-MS/MS on either an AB Sciex AP16500 QTrap or Waters TQ-S mass spectrometer using previously optimised analytical MRM (multiple reaction monitoring) methods, specific to the test compound.

The concentration of test compound in isolated samples was determined following analysis of the samples against the two replicates of the calibration line, injected before and after the sample set with an appropriate regression and weighting used. Only calibrators within ±15% of the expected test concentration value (±20% at the LLoQ) were included in the calibration line and any samples that fell outside of the limits of the calibration line were deemed to be less than or above the limit of quantification (LLoQ/ALoQ).

### Determination of Brain to Plasma Ratio and Free Brain Concentrations

Total CNS penetrance was calculated by dividing the concentration in the brain by the concentration in plasma for each timepoint. The mean brain to plasma ratio (Br:PI) was calculated by averaging these ratios (defining which timepoints were used).

The free drug hypothesis states that only unbound compound is able to interact with and elicit a pharmacological effect. Therefore, it is desirable for compounds to have a high free brain concentration. To calculate the free concentrations in each matrix, the determined concentrations are multiplied by the % free value as determined by plasma protein binding and brain tissue binding studies using rapid equilibrium dialysis. These values are then converted to molar concentrations to give a nanomolar free result at each timepoint.

The Kpuu is calculated as the ratio of free drug fraction unbound in brain to free drug unbound in plasma.

TABLE 14
Brain to plasma partitioning (Kpuu)

| | | | Dose | |
|---|---|---|---|---|
| Example | Formulation | Route | [mg/kg] | Kpuu |
| 3 | 30% HPBC in water solution, | IV | 1 | 0.56 |
| | pH adjusted to 4.5 with aq HCl | | | |
| 4 | 30% HPBC in water solution, | IV | 1 | 0.32 |
| | pH adjusted to 4.1 with aq HCl | | | |
| 5 | 10% NMP/90% HPBC (30% | IV | 1 | 0.02 |
| | w/v in saline) solution | | | |
| 9 | 10% NMP in 30% HPBC in | IV | 1 | 0.37 |
| | water solution, pH adjusted to | | | |
| | 4.2 with aq HCl | | | |
| 10 | 10% NMP/90% HPBC (30% | IV | 1 | 0.02 |
| | w/v in saline) solution | | | |
| 11 | 10% NMP/90% HPBC (30% | IV | 1 | 0.02 |
| | w/v in saline) solution | | | |
| 15 | 30% HPBC in water solution, | IV | 1 | 0.06 |
| | pH adjusted to 4.5 with aq HCl | | | |
| 17 | 10% NMP in 30% HPBC in | IV | 1 | 0.06 |
| | water solution, pH adjusted to | | | |
| | 4.0 with aq HCl | | | |
| 18 | 10% NMP in 30% HPBC in | IV | 1 | 0.03 |
| | water solution pH adjusted to | | | |
| | 4.0 with aq HCl | | | |
| 19 | 10% NMP in 30% HPBC in | IV | 1 | 0.05 |
| | water solution, pH adjusted to | | | |
| | 4.5 with aq HCl | | | |
| 20 | 10% NMP in 30% HPBC in | IV | 1 | 0.11 |
| | water solution, pH adjusted to | | | |
| | 4.5 with aq HCl | | | |

The data in the table above shows that the compounds of the invention, compounds of formulae (IA), (IB), (IIA), and (IIB), do not significantly penetrate the central nervous system.

In Vitro Incubations for reactive metabolite screen: GSH trapping study Each compound was incubated at a concentration of 10 μM with human liver microsomes (HLM, 1 mg/mL protein) or human liver S9 (1.5 mg/ml protein) in 100 mM phosphate buffer (pH 7.4) in the presence of NADPH (1 mM) and GSH (1 mM) as trapping agents to test for the formation of reactive metabolites. Incubations were performed for 0 (T0) and 60 (T60) min in a shaking incubator at 370C. The reaction was terminated by the addition of two-fold volume of 75% MeCN, vortexed, and centrifuged at 5-13K rpm for 5-10 min. The supernatant was analysed using LC-MS/MS with HRMS analysis. Formation of GSH conjugate peak is reported as % of parent compound peak at TO.

TABLE 15
GSH trapping study data for compounds of the invention

| | % GSH of adduct formation of |
|---|---|
| Example | parent |
| 4 | ≤0.30% |
| 10 | ≤0.30% |
| 17 | ≤0.30% |
| 18 | ≤0.30% |
| 20 | ≤0.30% |
| 31 | ≤0.30% |
| 41 | ≤0.30% |
| 42 | ≤0.30% |
| PF-02545920 | 0.62% |

The data in the table above demonstrates that the compounds of the invention, compounds of formulae (IA), (IB), (IIA), and (IIB), do not form a significant amount of reactive metabolite that may, for instance, cause idiosyncratic adverse drug reactions, often associated with drug-induced skin, liver and hematopoietic toxicities. The compounds of the invention are less susceptible reactive metabolite formation than PF-02545920.

### Assessing PDE10A Inhibitors for Use in the Treatment of Ulcerative Colitis

To explore the role of PDE10A in ulcerative colitis (UC) the Genotype-Tissue Expression (GTEx) database was used to look at PDE10A RNA expression in normal and diseased tissues. Alongside this, expression levels of guanylate cyclase 2C (GUCY2C) were also assessed. GUCY2C is an enzyme which synthesises cGMP in response to the endogenous peptides guanylin and uroguanylin as well as E. coli heat-stable enterotoxin.

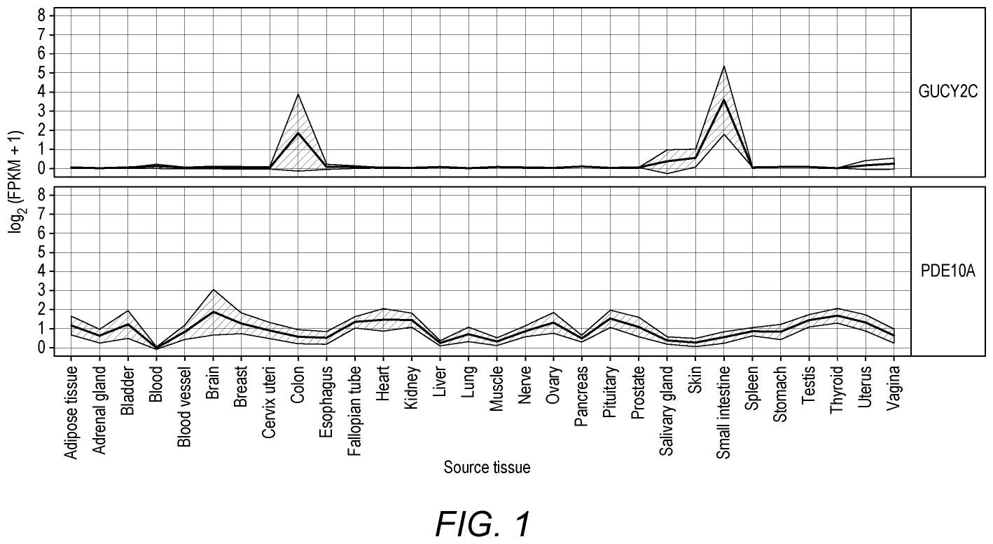

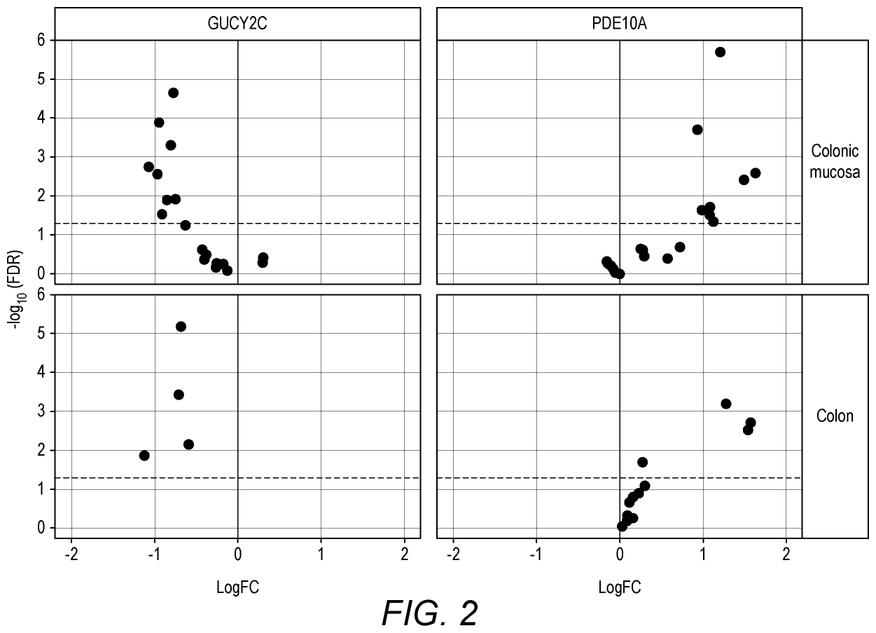

As previously described in the literature, in normal tissue PDE10A is expressed at low levels except in brain (as shown in FIG. 1). However in colonic mucosa and colon tissue from ulcerative colitis patients, PDE10A expression levels were significantly upregulated compared to normal controls (as shown in FIG. 2). This is a finding that has not been previously described in the literature and highlighting a potential undiscovered role for PDE10A in IBD pathology.

GUCY2C was seen to be specifically expressed at high levels in normal colon and small intestine (as shown in FIG. 1) suggesting a role for this enzyme in normal gut homeostasis. In UC colonic mucosa and colon, GUCY2C was significantly downregulated (as shown in FIG. 2), a finding that has previously been described in the literature.

Guanylate cyclase-C and cGMP signalling is downregulated in ulcerative colitis (Brenna et al. The guanylate cyclase-C signaling pathway is down-regulated in inflammatory bowel disease Scand J Gastroenterol. 50(10), 1241-52 (2015)) and decreases in expression of guanylate cyclase 2C, guanylin, and uroguanylin correlate with severity of disease. (Lan et al. Expression of guanylate cyclase-C, guanylin, and uroguanylin is downregulated proportionally to the ulcerative colitis disease activity index Sci Rep. 6, 25034, (2016) published online 29 Apr. 2016 doi: 10.1038/srep25034). This suggests that reduced cGMP signalling plays a role in UC pathology. cGMP in the GI tract has also been shown to play a role in fluid and electrolyte secretion, barrier function, inflammation and proliferation (Waldman et al. Guanylate cyclase-C as a therapeutic target in gastrointestinal disorders., Gut. 67(8), 1543-1552 (2018)).

While less studied in inflammation than cAMP, reduced cGMP signalling has also been shown to increase inflammation in other systems (Ahluwalia et al. Antiinflammatory activity of soluble guanylate cyclase: cGMP-dependent down-regulation of P-selectin expression and leukocyte recruitment. Proc Natl Acad Sci U S A. 101(5), 1386-91 (2004); Raposo et al. Role of iNOS-NO-cGMP signaling in modulation of inflammatory and myelination processes. Brain Res Bull. 104, 60-73 (2014)).

Taken together, in UC colon and colonic mucosa, cGMP hydrolysing activity by PDE10A would be increased and cGMP synthesizing activity by guanylate cyclase 2C would be decreased resulting in a net decrease in cGMP levels and signalling.

The therapeutic potential of inhibitors of PDE10A to treat inflammatory bowel diseases was assessed using tissue samples from inflamed colonic mucosa from ulcerative colitis patients.

The effect of selective PDE10A inhibition was tested on inflamed colonic mucosa from ulcerative colitis patients taken during routine endoscopy (Protocol 1 detailed below). These samples retain a disease phenotype in ex-vivo culture, secrete high basal levels of inflammatory cytokines, and represent a highly relevant and translational disease model. The effect of the PDE10A inhibitors on levels of the inflammatory cytokines IL-6 and IL-8 released from these tissue samples were measured. Both IL-6 and IL-8 are key regulators in ulcerative colitis pathology and their levels correlate with disease severity (Waldner M J et al. Master regulator of intestinal disease: IL-6 in chronic inflammation and cancer development. Semin Immunol. 26(1), 75-9 (2014); Bernardo D et al. IL-6 promotes immune responses in human ulcerative colitis and induces a skin-homing phenotype in the dendritic cells and T-cells they stimulate. Eur J Immunol. 42(5), 1337-53 (2012); Pearl D S, Cytokine mucosal expression in ulcerative colitis, the relationship between cytokine release and disease activity. J Crohns Colitis. 7(6), 481-9 (2013)).

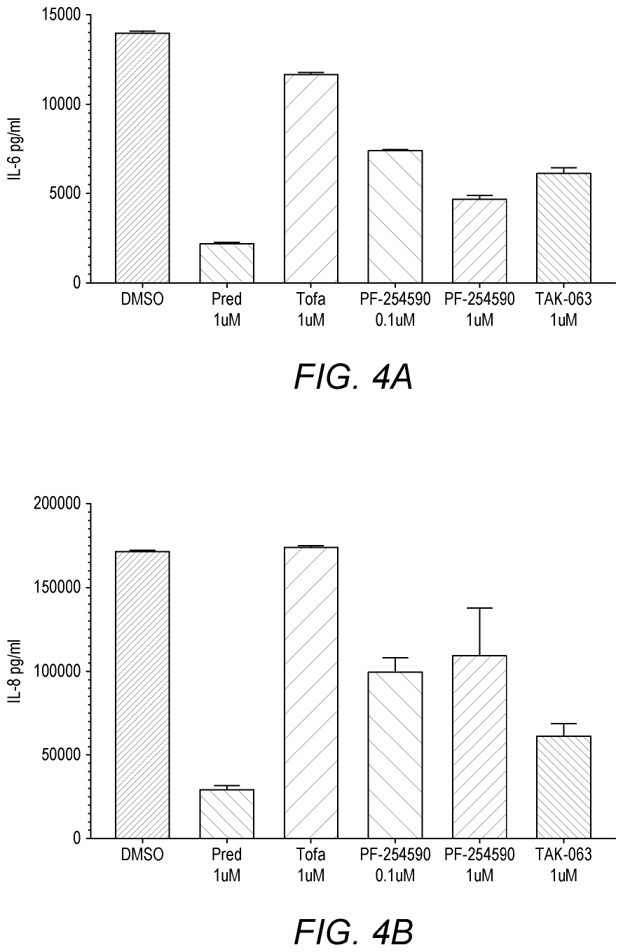

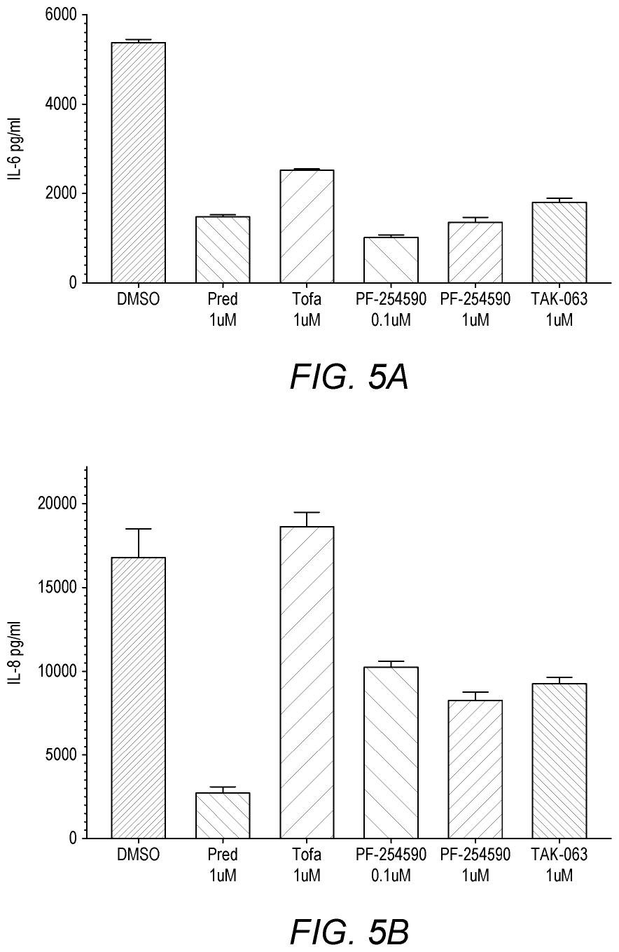

The structurally distinct PDE10A inhibitors PF-02545920 and TAK-063 were tested alongside two positive control compounds, the steroid prednisolone and the Janus kinase inhibitor tofacitinib, in colon biopsy samples from two ulcerative colitis patients. These colon biopsies retain an inflammatory phenotype and secrete high levels of inflammatory cytokines in ex-vivo culture. Selective PDE10A inhibition significantly reduced the secreted levels of IL-6 and IL-8 when compared to the DMSO vehicle (FIGS. 4 and 5). This reduction was comparable to that seen with the positive controls. PF-02545920 was tested at concentrations of 0.1 μM and 1 μM. TAK-063 was tested at a concentration of 1 uM. The doses tested of each inhibitor will result in selective PDE10A inhibition over the other PDE family members.

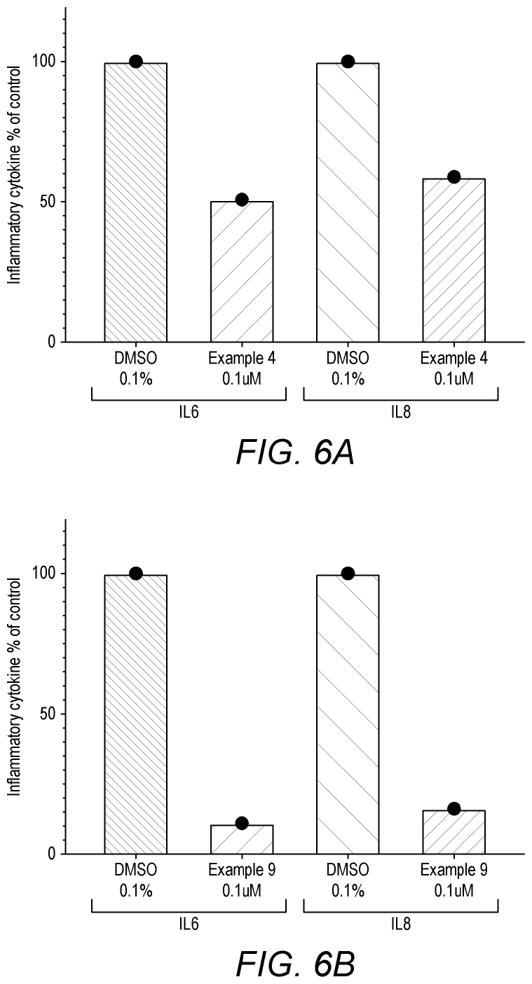

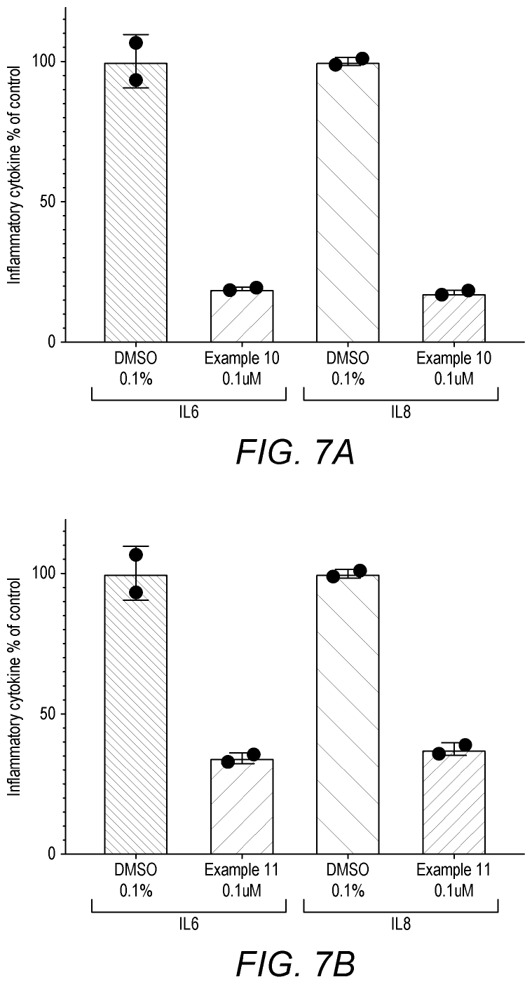

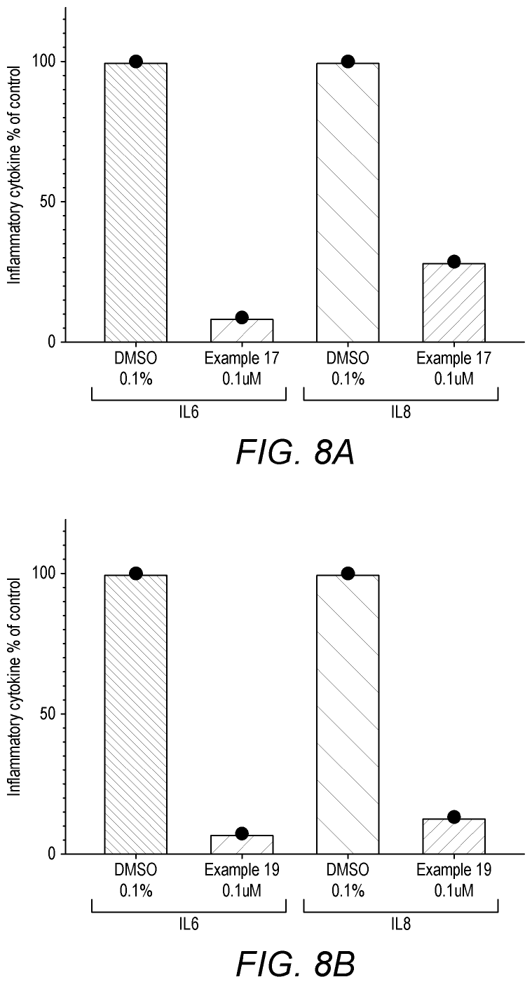

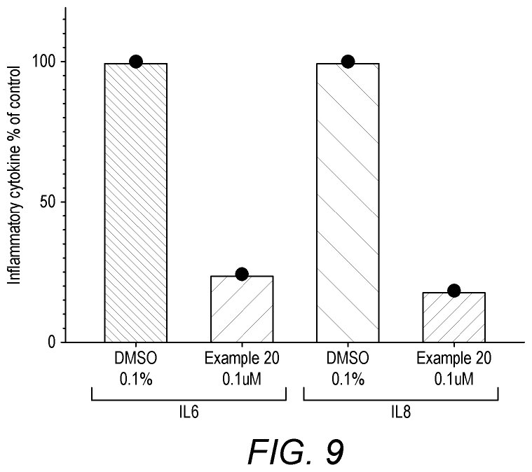

The compounds of the invention in Examples 4, 9, 10, 11, 17, 19 and 20 were tested at a concentration of 100 nM (concentration selective for PDE10A inhibition), and were found to significantly reduce the secreted levels of IL-6 and IL-8 when compared to the vehicle control. The ability of selective PDE10A inhibition by compounds of the invention to significantly reduce levels of pathologic inflammatory cytokines in ex-vivo UC patient-derived colon tissue demonstrates the therapeutic utility of PDE10A inhibitors for the treatment of UC. The results are shown in FIGS. 6 to 9.

The structure of PF-02545920 is below. PF-02545920 is a potent and selective cyclic nucleotide PDE10A competitive inhibitor with a reported IC₅₀ value of 1.26 nM. PF-02545920 has been investigated in clinical trials for the treatment of Huntington's Disease. Patients were given 5 or 20 mg of PF-02545920 twice daily.

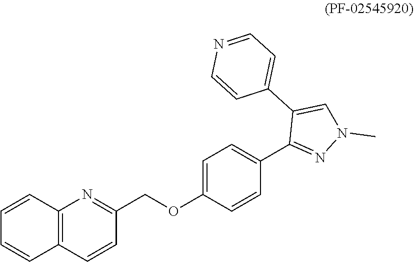 [SMILES: Cn1cc(-c2ccncc2)c(-c2ccc(OCc3ccc4ccccc4n3)cc2)n1]

In isolated enzyme biochemical assays, PF-02545920 has been shown to be a highly selective PDE10A inhibitor with an IC₅₀ for PDE10A <5 nM and IC₅₀s for other PDE family members >1 μM (Grauer S M et. al. Phosphodiesterase 10A inhibitor activity in preclinical models of the positive, cognitive, and negative symptoms of schizophrenia. J Pharmacol Exp Ther. 2009 331(2), 574-90). Therefore, at the test concentration of 0.1 μM and 1 μM in an ex-vivo tissue assay, PF-02545920 will selectively inhibit PDE10A.

The structure of TAK-063 below. TAK-063 was studied in a phase 2 clinical trial for the treatment of people with schizophrenia. TAK-063 was given at 20 mg once per day but may be reduced to 10 mg once per day if the higher dose was intolerable.

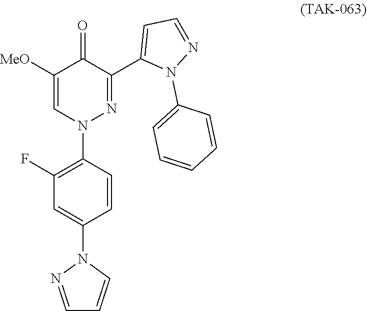 [SMILES: COc1cn(-c2ccc(-n3cccn3)cc2F)nc(-c2ccnn2-c2ccccc2)c1=O]

In isolated enzyme biochemical assays, TAK-063 has been shown to be a highly selective PDE10A inhibitor with an IC₅₀ for PDE10A of 0.3 nM and IC₅₀s for other PDE family members >5 μM (Kunitomo J et. al. Discovery of 1-[2-fluoro-4-(1H-pyrazol-1-yl)phenyl]-5-methoxy-3-(1-phenyl-1H-pyrazol-5-yl)pyridazin-4(1H)-one (TAK-063), a highly potent, selective, and orally active phosphodiesterase 10A (PDE10A) inhibitor. J Med. Chem. 57(22), 9627-43 (2014)) Therefore, at the test concentration of 1 uM in an ex-vivo tissue assay, TAK-063 will selectively inhibit PDE10A.

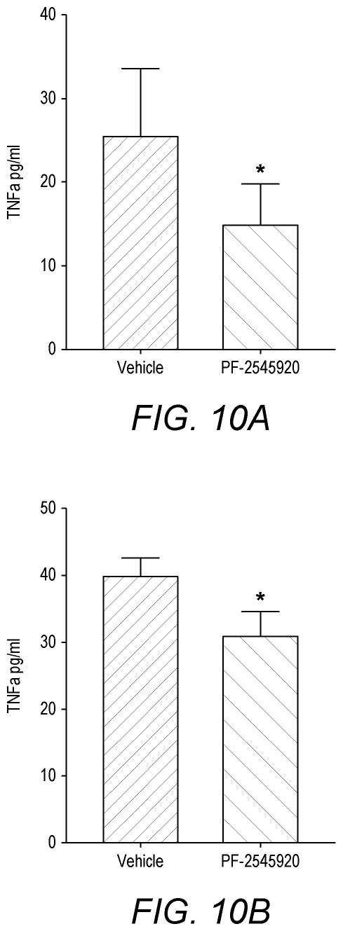

The effect of selective PDE10A inhibition was also tested on inflamed colonic mucosa from pharmacotherapy treatment-refractory ulcerative colitis patients taken during colon resection surgery (Protocol 2 detailed below). The PDE10A inhibitor PF-02545920 (1 μM) was tested in colon samples from two ulcerative colitis patients. The effect of selective PDE10A inhibition on levels of the inflammatory cytokine TNFα released from these tissue samples were measured. TNFα is a pro-inflammatory mediator that is expressed at high levels in the colonic mucosa of patients with UC and is the target of anti-TNFα biologics which have demonstrated efficacy in the treatment of UC (Pugliese D. et al. Anti TNF-α therapy for ulcerative colitis: current status and prospects for the future., Expert Rev Clin Immunol. 13(3), 223-233 (2017)). Selective PDE10A inhibition significantly reduced the secreted levels of TNFα compared to the DMSO vehicle (FIG. 10).

The ability of selective PDE10A inhibition to significantly reduce levels of inflammatory cytokines in UC patient-derived colonic mucosa demonstrates the therapeutic utility of PDE10A inhibitors for the treatment of UC.

### Assessing PDE10A Inhibitors for Use in the Treatment of Crohn's Disease

As mentioned above, treatments for UC should also be viable treatments for CD. In particular, it has been shown that cGMP signalling is reduced in both UC and CD (Brenna, et al. The guanylate cyclase-C signaling pathway is down-regulated in inflammatory bowel disease. Scand J Gastroentero 50, 1241-1252 (2015)) a mechanism that is highly relevant to PDE10A.

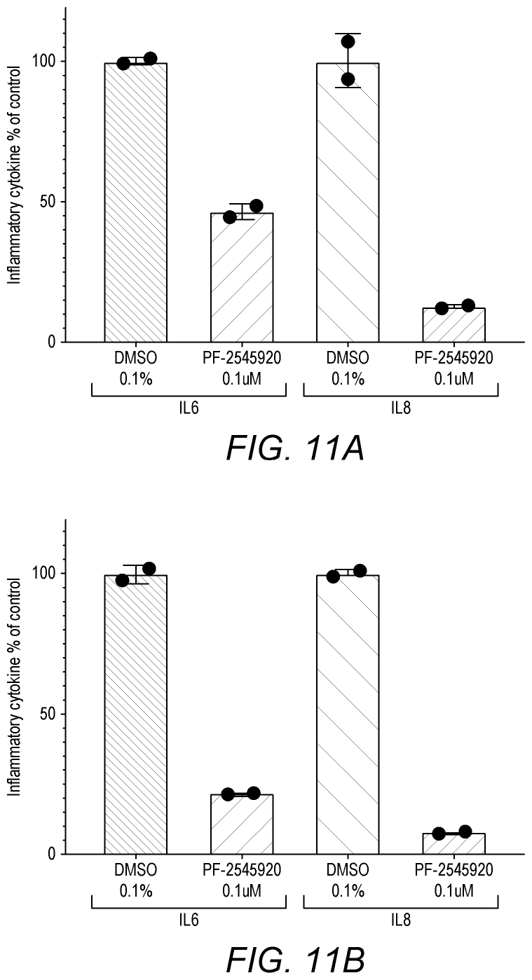

In addition, we tested the effect of selective PDE10A inhibition using 0.1 uM PF-2545920 on inflamed colonic mucosa from Crohn's Disease patients taken during routine endoscopy (Protocol 1). Selective PDE10A inhibition significantly reduced the secreted levels of IL-6 and IL-8 from 2 independent CD patient biopsies when compared to the DMSO vehicle (FIG. 11).

The ability of selective PDE10A inhibition to significantly reduce levels of inflammatory cytokines in CD patient-derived colonic mucosa demonstrates the therapeutic utility of PDE10A inhibitors for the treatment of CD in addition to UC. As PF-2545920 may treat CD by inhibition of PDE10A, the compounds of the invention may also treat CD.

### Protocol 1

Biopsy tissue was obtained from inflamed colonic mucosa from ulcerative colitis or Crohn's disease patients during routine endoscopy. Ex-vivo biopsy cultures for the analysis of inflammatory cytokine biomarkers were run as previously described (Vossenkämper A. et al. A CD3- specific antibody reduces cytokine production and alters phosphoprotein profiles in intestinal tissues from patients with inflammatory bowel disease. Gastroenterology, 147, 172-183 (2014)). Biopsies were incubated in organ culture for 24 h with the addition of positive control compounds, or specific PDE10A inhibitors. Supernatants collected at the end of the experiment were snap-frozen and stored at −70° C. For the measurement of cytokines, the frozen culture supernatants were thawed and analysed for levels of the inflammatory cytokines using Luminex cytokine assay kits (R&D Systems) and an R&D Systems MAGPIX® analyser. Mean values ±SDs were calculated for the levels of spontaneous cytokine production measured in biopsy culture supernatants from each treatment group.

### Protocol 2

Ulcerative colitis donor samples were obtained with full ethical consent from patients undergoing therapeutic resection for ulcerative colitis. Tissues were placed apical (mucosal) side facing upwards on a Netwell filter. The biopsies were then cultured in either control media or media containing the test compound in an incubator at 37° C. and high O₂ atmospheric conditions. To try to minimize variation, the biopsies were also cultured in the presence of the inflammatory stimulant Staphylococcal Enterotoxin B (SEB) to help normalise cytokine levels. At approximately 18 hours post-culture start, media samples were collected, protease inhibitor added and samples stored at −80° C. Supernatants were subsequently subjected to ELISA analysis for cytokine measurement.

### Assessing PDE10A Inhibitors in IL-8 Neutrophil Activation

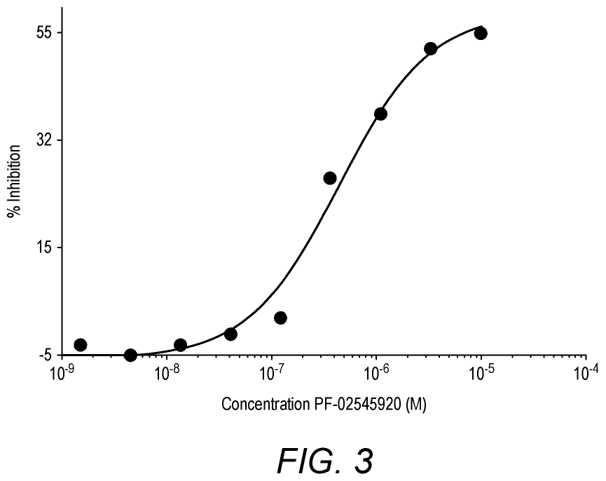

The PDE10A compound PF-02545920 was assessed in an in vitro assay of IL-8 neutrophil activation. PF-02545920 dose dependently inhibited IL-8 induced neutrophil activation (as shown in FIG. 3). This was of interest as a role for PDE10A in neutrophil function had not been previously described and further suggested a role for PDE10A in modulating inflammation and that a PDE10A inhibitor would be suitable as a therapeutic for inflammatory bowel diseases.

The forgoing embodiments are not intended to limit the scope of the protection afforded by the claims, but rather to describe examples of how the invention may be put into practice.

1. A compound of Formula (IA) or (IB) 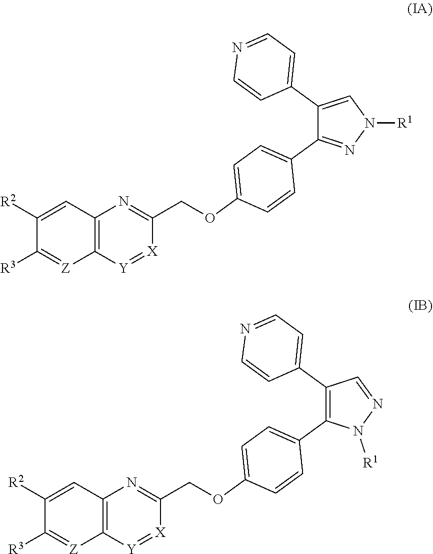
or a pharmaceutically acceptable salt, solvate, hydrate, tautomer, optical isomer,
N-oxide, and/or prodrug thereof, wherein
X is selected from N and CR⁴;
Y is selected from N and CR⁵;
and at least one of CR⁴ and CR⁵ is present;
Z is selected from N and CR⁶;
R¹ is selected from the group consisting of H, C₁-C₆ alkyl and —SO₂R⁷, wherein the C₁-C₆ alkyl is optionally substituted with one or more substituents independently selected from halo, oxo, —NR^{a}R^{b}, —C(O)NR^{a}R^{b}, —C(O)OR, —OR^{c};
R² and R³ are independently selected from group consisting of H, halo, and C₁-C₆ alkyl, wherein the C₁-C₆ alkyl is optionally substituted with one or more halo atoms;
R⁴ and R⁵ are independently selected from group consisting of H, —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e}, 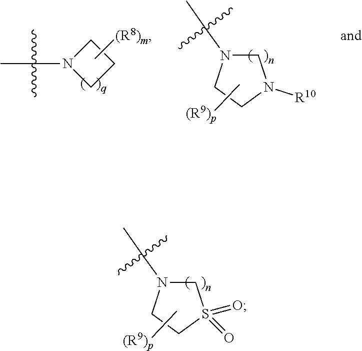
R⁶ is selected from H and C₁-C₆ alkyl, wherein the C₁-C₆ alkyl is optionally substituted with one or more halo atoms;
R⁷ is selected from H and C₁-C₆ alkyl, wherein the C₁-C₆ alkyl is optionally substituted with one or more halo atoms;
R⁸ is selected from C₁-C₆ alkyl, —OH, and —NR^{a}R^{b}, wherein the C₁-C₆ alkyl is optionally substituted with one or more halo atoms;
R⁹ is selected from C₁-C₆ alkyl, —OH, oxo, and —NR^{a}R^{b}, wherein the C₁-C₆ alkyl is optionally substituted with one or more halo atoms;
R¹⁰ is selected from H and C₁-C₆ alkyl, wherein the C₁-C₆ alkyl is optionally substituted with one or more halo atoms;
each R^{a}, R^{b}, R^{c}, R^{d} and R^{e} are independently selected from H and C₁-C₆ alkyl wherein the C₁-C₆ alkyl is optionally substituted with one or more halo atoms,
or R^{a} and R^{b} can be taken together with the nitrogen atom to which they are attached to form a 5- or 6-membered heterocycle,
or two R^{e} groups attached to the same atom can be taken together with the atom to which they are attached to form a 5- or 6-membered heterocycle;
m is 0, 1, 2, 3 or 4
n is 1 or 2;
p is 0, 1, 2, 3 or 4; and
q is 0, 1, 2, 3 or 4,
wherein when R¹ is H or optionally substituted C₁-C₆ alkyl, then at least one of R⁴ and R⁵ is present and not H.

2. The compound as claimed in claim 1, wherein at least one of R⁴ and R⁵ is present and is selected from the group consisting of —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e},

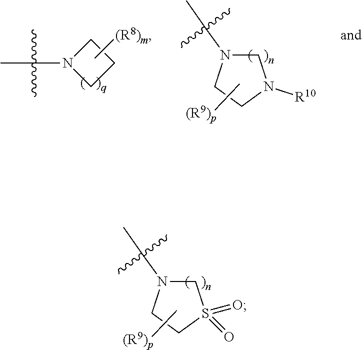

3. The compound as claimed in claim 1 or claim 2, wherein R² is H.

4. The compound as claimed in any preceding claim, wherein R³ is H

5. The compound as claimed in any preceding claim, wherein R¹ is selected from the group consisting of H, C₁-C₃ alkyl and —SO₂Me, wherein the C₁-C₃ alkyl is optionally substituted with one or more substituents independently selected from halo and —C(O)OH.

6. The compound as claimed in any preceding claim, wherein R¹ is selected from the group consisting of H, Me, Et, —CH₂CF₃, CH₂C(O)OH, cyclopropyl, and —SO₂Me.

7. The compound as claimed in any preceding claim, wherein Z is selected from N and CR⁶, and R⁶ is selected from H, F, Cl and Me; preferably wherein Z is CR⁶.

8. The compound as claimed in any preceding claim, wherein each R^{d} is selected from H and Me, and each R^{e} is Me.

9. The compound as claimed in any preceding claim, wherein X is selected from N and CR⁴; and R⁴ is selected from H, —C(O)OR^{c}, —C(O)N(R^{d})SO₂R^{e}; —C(O)N═S(O)R^{e}₂;

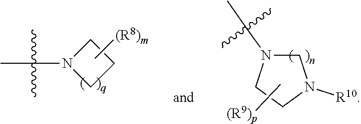

10. The compound as claimed in any preceding claim, wherein X is selected from N and CR⁴; and R⁴ is selected from H, —C(O)OH, —C(O)NHSO₂Me, —C(O)NMeSO₂Me, —C(O)N═S(O)Me₂,

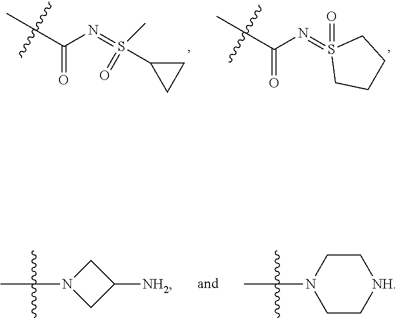

11. The compound as claimed in any preceding claim, wherein Y is selected from N and CR⁵, and R⁵ is selected from H, —C(O)OH, —C(O)N(Me)SO₂Me, —C(O)N═S(O)Me₂, —N═S(O)Me₂, —NHC(O)N═S(O)Me₂, —NHC(O)NHMe, —NHSO₂Me, —S(O)(═NH)Me,

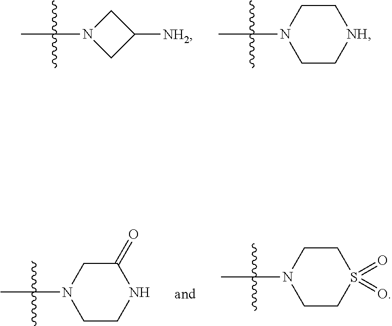 [SMILES: *N1CC(N)C1 ; *N1CCNCC1 ; *N1CCNC(=O)C1 ; *N1CCS(=O)(=O)CC1]

12. The compound as claimed in any preceding claim, wherein the compound is selected from the group consisting of
2-[[4-[2-Methyl-4-(4-pyridyl)pyrazol-3-yl]phenoxy]methyl]quinoline-4-carboxylic acid;
2-[[4-[2-Methyl-4-(4-pyridyl)pyrazol-3-yl]phenoxy]methyl]quinoline-3-carboxylic acid;
2-[[4-[1-Methyl-4-(4-pyridyl)pyrazol-3-yl]phenoxy]methyl]quinoline-3-carboxylic acid;
Ammonium 2-[[4-[1-methyl-4-(4-pyridyl)pyrazol-3-yl]phenoxy]methyl]quinoline-4-carboxylate;
2-[4-(4-Pyridyl)-3-[4-(2-quinolylmethoxy)phenyl]pyrazol-1-yl]acetic acid;
2-[[4-[1-Methylsulfonyl-4-(4-pyridyl)pyrazol-3-yl]phenoxy]methyl]quinoline;
N-[Dimethyl(oxo)-λ6-sulfanylidene]-5-methyl-2-[[4-[1-methyl-4-(4-pyridyl)pyrazol-3-yl]phenoxy]methyl]quinoline-3-carboxamide;
N-[Dimethyl(oxo)-λ6-sulfanylidene]-2-[[4-[4-(4-pyridyl)-1-(2,2,2-trifluoroethyl)pyrazol-3-yl]phenoxy]methyl]quinoline-4-carboxamide:
2-[[4-[1-Methyl-4-(4-pyridyl)pyrazol-3-yl]phenoxy]methyl]-N-methylsulfonyl-quinoline-3-carboxamide;
N-[Dimethyl(oxo)-λ6-sulfanylidene]-2-[[4-[1-methyl-4-(4-pyridyl)pyrazol-3-yl]phenoxy]methyl] quinoline-3-carboxamide;
N-[Dimethyl(oxo)-λ6-sulfanylidene]-2-[[4-[1-methyl-4-(4-pyridyl)pyrazol-3-yl]phenoxy]methyl] quinoline-4-carboxamide;
N-[Dimethyl(oxo)-λ6-sulfanylidene]-2-[[4-[4-(4-pyridyl)-1H-pyrazol-3-yl]phenoxy]methyl] quinoline-4-carboxamide;
2-[[4-[1-Methyl-4-(4-pyridyl)pyrazol-3-yl]phenoxy]methyl]-N-(1-oxothiolan-1-ylidene)quinoline-3-carboxamide;
N-(Cyclopropyl-methyl-oxo-λ6-sulfanylidene)-2-[[4-[1-methyl-4-(4-pyridyl)pyrazol-3-yl]phenoxy]methyl] quinoline-3-carboxamide;
N-Methyl-2-[[4-[1-methyl-4-(4-pyridyl)pyrazol-3-yl]phenoxy]methyl]-N-methylsulfonyl-quinoline-3-carboxamide;
N-Methyl-2-[[4-[1-methyl-4-(4-pyridyl)pyrazol-3-yl]phenoxy]methyl]-N-methylsulfonyl-quinoline-4-carboxamide;
N-[Dimethyl(oxo)-λ6-sulfanylidene]-2-[[4-[1-methyl-4-(4-pyridyl)pyrazol-3-yl]phenoxy]methyl]quinazoline-4-carboxamide;
N-[Dimethyl(oxo)-λ6-sulfanylidene]-3-[[4-[1-methyl-4-(4-pyridyl)pyrazol-3-yl]phenoxy]methyl] quinoxaline-2-carboxamide;
N-[Dimethyl(oxo)-λ6-sulfanylidene]-2-[[4-[1-methyl-4-(4-pyridyl)pyrazol-3-yl]phenoxy]methyl]-1,5-naphthyridine-3-carboxamide;
N-[Dimethyl(oxo)-λ6-sulfanylidene]-7-fluoro-2-[[4-[1-methyl-4-(4-pyridyl)pyrazol-3-yl]phenoxy]methyl]quinoline-3-carboxamide;
N-[Dimethyl(oxo)-λ6-sulfanylidene]-6-fluoro-2-[[4-[1-methyl-4-(4-pyridyl)pyrazol-3-yl]phenoxy]methyl]quinoline-3-carboxamide;
N-[Dimethyl(oxo)-λ6-sulfanylidene]-5-fluoro-2-[[4-[1-methyl-4-(4-pyridyl)pyrazol-3-yl]phenoxy]methyl]quinoline-3-carboxamide;
N-[Dimethyl(oxo)-λ6-sulfanylidene]-6-methyl-2-[[4-[1-methyl-4-(4-pyridyl)pyrazol-3-yl]phenoxy]methyl]quinoline-3-carboxamide;
N-[Dimethyl(oxo)-λ6-sulfanylidene]-6-fluoro-2-[[4-[1-methyl-4-(4-pyridyl)pyrazol-3-yl]phenoxy]methyl]quinoline-4-carboxamide:
5-Chloro-N-[Dimethyl(oxo)-λ6-sulfanylidene]-2-[[4-[1-methyl-4-(4-pyridyl)pyrazol-3-yl]phenoxy]methyl]quinoline-4-carboxamide;
N-[Dimethyl(oxo)-λ6-sulfanylidene]-2-[[4-[1-ethyl-4-(4-pyridyl)pyrazol-3-yl]phenoxy]methyl]quinoline-4-carboxamide;
2-[[4-[1-Cyclopropyl-4-(4-pyridyl)pyrazol-3-yl]phenoxy]methyl]-N-[dimethyl(oxo)-λ6-sulfanylidene]quinoline-4-carboxamide;
2-[[4-[4-(4-Pyridyl)-1H-pyrazol-3-yl]phenoxy]methyl]quinoline-3-carboxylic acid;
2-[[4-[4-(4-Pyridyl)-1H-pyrazol-3-yl]phenoxy]methyl]quinoline-4-carboxylic acid;
N-[Dimethyl(oxo)-λ6-sulfanylidene]-2-[[4-[4-(4-pyridyl)-1H-pyrazol-3-yl]phenoxy]methyl]quinoline-3-carboxamide;
Dimethyl-oxo-[[2-[[4-[4-(4-pyridyl)-1H-pyrazol-3-yl]phenoxy]methyl]-4-quinolyl]imino]-λ6-sulfane;
Imino-methyl-oxo-[2-[[4-[4-(4-pyridyl)-1H-pyrazol-3-yl]phenoxy]methyl]-4-quinolyl]-λ6-sulfane;
N-[2-[[4-[1-Methyl-4-(4-pyridyl)pyrazol-3-yl]phenoxy]methyl]-4-quinolyl]methanesulfonamide;
1-Methyl-3-[2-[[4-[1-methyl-4-(4-pyridyl)pyrazol-3-yl]phenoxy]methyl]-4-quinolyl]urea;
1-[Dimethyl(oxo)-λ6-sulfanylidene]-3-[2-[[4-[1-methyl-4-(4-pyridyl)pyrazol-3-yl]phenoxy]methyl]-4-quinolyl]urea;
4-[2-[[4-[1-Methyl-4-(4-pyridyl)pyrazol-3-yl]phenoxy]methyl]quinazolin-4-yl]-1,4-thiazinane 1,1-dioxide;
4-[2-[[4-[1-Methyl-4-(4-pyridyl)pyrazol-3-yl]phenoxy]methyl]quinazolin-4-yl]piperazin-2-one;
1-[2-[[4-[1-Methyl-4-(4-pyridyl)pyrazol-3-yl]phenoxy]methyl]quinazolin-4-yl]azetidin-3-amine;
2-[[4-[1-Methyl-4-(4-pyridyl)pyrazol-3-yl]phenoxy]methyl]-4-piperazin-1-yl-quinazoline;
1-[3-[[4-[1-Methyl-4-(4-pyridyl)pyrazol-3-yl]phenoxy]methyl]quinoxalin-2-yl]azetidin-3-amine; and
2-[[4-[1-Methyl-4-(4-pyridyl)pyrazol-3-yl]phenoxy]methyl]-3-piperazin-1-yl-quinoxaline,
or a pharmaceutically acceptable salt, solvate, hydrate, tautomer, optical isomer, N-oxide, and/or prodrug thereof.

13. A compound of Formula (IIA) or (IIB) 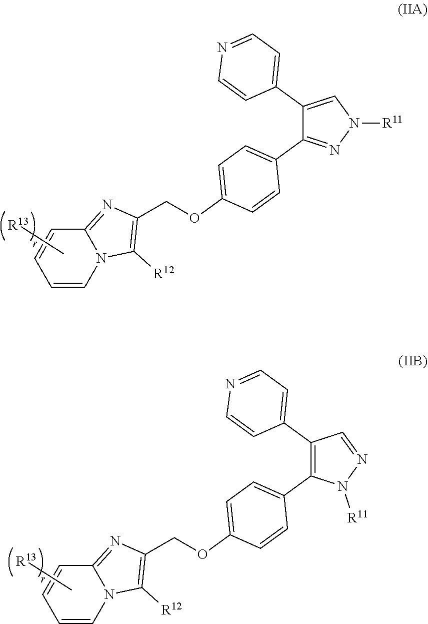
or a pharmaceutically acceptable salt, solvate, hydrate, tautomer, optical isomer, N-oxide, and/or prodrug thereof, wherein
R¹¹ is selected from the group consisting of H, C₁-C₆ alkyl and —SO₂R⁷, wherein the C₁-C₆ alkyl is optionally substituted with one or more substituents independently selected from halo, oxo, —NR^{a}R^{b}, —C(O)NR^{a}R^{b}, —C(O)OR^{c}, —OR^{c}, preferably is it selected from the group consisting of H and C₁-C₆ alkyl, more preferably C₁-C₆ alkyl, even more preferably Me, wherein the C₁-C₆ alkyl or Me is optionally substituted with one or more halo, preferably F,
R¹² is selected from the group consisting of H, —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e}, 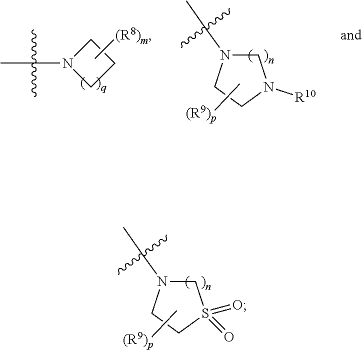
preferably —C(O)N═S(O)R^{e}₂, more preferably —C(O)N═S(O)Me₂;
R¹³ is selected from the group consisting of halo, —OR^{f}, and C₁-C₆ alkyl, wherein the C₁-C₆ alkyl is optionally substituted with one or more halo, preferably F;
R^{f} is selected from the group consisting of H and C₁-C₆ alkyl, wherein the C₁-C₆ alkyl is optionally substituted with one or more halo, preferably F; and
r is 0, 1, 2, 3, or 4, preferably 0; and
wherein R⁷, R⁸, R⁹, R¹⁰, R^{a}, R^{b}, R^{c}, R^{d}, R^{e}, m, n, p, and q are as defined in any preceding claim.

14. The compound as claimed in claim 13, wherein R¹² is selected from the group consisting of —C(O)OR^{c}, —C(O)N(R^{d})SO₂R^{e}, —C(O)N═S(O)R^{e}₂, —N═S(O)R^{e}₂, —N(R^{d})C(O)N═S(O)R^{e}₂, —N(R^{d})C(O)NR^{e}₂, —N(R^{d})SO₂R^{e}, —S(O)(═NR^{d})R^{e}, 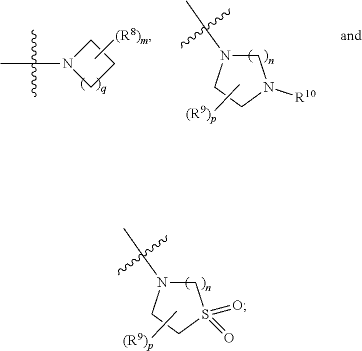
preferably —C(O)N═S(O)R^{e}₂, more preferably —C(O)N═S(O)Me₂.

15. The compound as claimed in claim 13 or claim 14, wherein the compound is N-[dimethyl(oxo)-λ6-sulfanylidene]-2-[[4-[1-methyl-4-(4-pyridyl)pyrazol-3-yl]phenoxy]methyl]imidazo[1,2-a]pyridine-3-carboxamide, or a pharmaceutically acceptable salt, solvate, hydrate, tautomer, optical isomer, N-oxide, and/or prodrug thereof

16. A pharmaceutical composition comprising a compound as defined in any one of claims 1 to 15 and one or more excipients.

17. A compound as defined in any one of claims 1 to 15, or a pharmaceutical composition as defined in claim 15, for use as a medicament.

18. A compound as defined in any one of claims 1 to 15, or the pharmaceutical composition as defined in claim 16, for use in the prevention and/or treatment of an inflammatory bowel disease.

19. The compound or pharmaceutical composition for use as claimed in claim 18, wherein the inflammatory bowel disease is ulcerative colitis and/or Crohn's disease.

20. A method for the prevention and/or treatment of a disease or condition comprising administering to a subject a compound as defined in any one of claims 1 to 15, wherein the disease or condition is susceptible to PDE10A inhibition.

21. The method as claimed in claim 20, wherein the disease or condition is an inflammatory bowel disease.

22. The method as claimed in claim 21, wherein the inflammatory bowel disease is ulcerative colitis and/or Crohn's disease.

23. Use of a compound as defined in any one of claims 1 to 15 for the manufacture of a medicament.

24. The use as claimed in claim 23, wherein the medicament is for the prevention and/or treatment of an inflammatory bowel disease.

25. The use as claimed in claim 24, wherein the inflammatory bowel disease is ulcerative colitis and/or Crohn's disease.
